(19) United States
(12) Patent Application Publication
(10) Pub. No.: US 2016/0333043 A1
(43) Pub. Date: Nov. 17, 2016

(54) E-Selectin Antagonists Modified By Macrocycle Formation to the Galactose

(71) Applicant: GLYCOMIMETICS, INC., Gaithersburg, MD (US)

(72) Inventors: John L. MAGNANI, Gaithersburg, MD (US); John M. PETERSON, Slate Hill, NY (US); Mirko ZIERKE, Radolfzell (DE); Martin SMIESKO, Allschwill (CH); Beat ERNST, Magden (CH)

(73) Assignee: GLYCOMIMETICS, INC., Rockvill, MD (US)

(21) Appl. No.: 15/112,058

(22) PCT Filed: Jan. 15, 2015
(86) PCT No.: PCT/US2015/011523

§ 371 Date: Jul. 15, 2016

Related U.S. Application Data
(60) Provisional application No. 61/928,778, filed on Jan. 17, 2014.

(51) Int. Cl. C07H 9/02

(52) U.S. Cl. CPC C07H 9/02

(57) ABSTRACT

Provided herein are glycomimetic E-selectin antagonist compounds of formula (I)) and pharmaceutical compositions comprising at least one of the same. The compounds of the present disclosure include trisaccharide domain mimics comprising at least one macrocycle created through the 2^{nd} and 3^{rd} positions on a galactose within the mimic. Methods are also provided comprising using at least one of such compounds and compositions comprising at least one of the same to treat and/or prevent diseases and disorders treatable by inhibiting binding of an E-selectin to an E-selectin ligand.

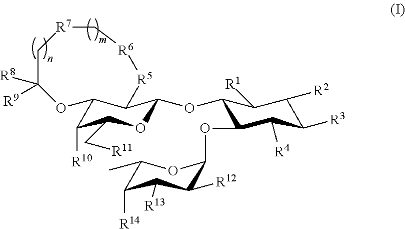

### CROSS REFERENCE TO RELATED APPLICATIONS

This application claims the benefit under 35 U.S.C. §119(e) to U.S. Provisional Application No. 61/928,778 filed Jan. 17, 2014, which application is incorporated by reference herein in its entirety.

### FIELD OF INVENTION

The present disclosure relates to glycomimetic E-selectin antagonists, pharmaceutically acceptable salts, and prodrugs thereof, as well as to pharmaceutical compositions containing the same and to their use for treating and/or preventing diseases, disorders, and/or conditions associated with E-selectin activity including, for example, inflammatory diseases and cancer.

### BACKGROUND OF THE INVENTION

When a tissue is infected or damaged, selectins are involved in directing leukocytes and other immune system components to the site of inflammation (see, e.g., McEver et al., J. Biol. Chem. 270:11025-28 (1995)). Although the leukocyte extravasation to infected or damaged tissue is critical for mounting an effective immune defense, excessive or inappropriate leukocyte accumulation may result in injury to the host tissues instead of repair (see, e.g., Bevilacqua et al., Annu. Rev. Med. 45:361-78 (1994)). Agents and compositions that modulate the leukocyte-endothelial cell adhesion process may therefore be beneficial to the treatment of, for example, autoimmune and inflammatory diseases.

In addition, recent investigations have suggested that cancer cells are immunostimulatory and interact with selectins to extravasate and metastasize (see, e.g., Gout et al., Clin. Exp. Metastasis 25:335-344 (2008); Kannagi et al., Cancer Sci. 95:377-84 (2004); Witz, Immunol. Lett. 104:89-93 (2006); Brodt et al., Int. J. Cancer 71:612-19 (1997)). Interfering with these interactions may therefore be desirable and lead to new cancer therapies.

Selectins are a group of structurally similar cell surface receptors that are important for mediating leukocyte binding to endothelial cells. These proteins are type 1 membrane proteins and are composed of an amino terminal lectin domain, an epidermal growth factor (EGF)-like domain, a variable number of complement receptor related repeats, a hydrophobic membrane spanning domain and a cytoplasmic domain. The binding interactions appear to be mediated by contact of the lectin domain of the selectins and various carbohydrate ligands.

There are three known selectins: E-selectin, P-selectin, and L-selectin. E-selectin is found on the surface of activated endothelial cells, which line the interior wall of capillaries. E-selectin binds to the carbohydrate sialyl-Lewis^{x} (sLe^{x}), which is presented as a glycoprotein or glycolipid on the surface of certain leukocytes (monocytes and neutrophils) and helps these cells adhere to capillary walls in areas where surrounding tissue is infected or damaged; and E selectin also binds to sialyl-Lewis^{a} (sLe^{a}), which is expressed on many tumor cells. P-selectin is expressed on inflamed endothelium and platelets, and also recognizes sLe^{x} and sLe^{a}, but also contains a second site that interacts with sulfated tyrosine. The expression of E-selectin and P-selectin is generally increased when the tissue adjacent to a capillary is infected or damaged. L-selectin is expressed on leukocytes. Selectin-mediated intercellular adhesion is an example of a selectin-mediated function.

Despite its ability to bind to E-selectin, therapeutic application of the common tetrasaccharide epitope, sLe^{x}, has proven unsuccessful due in part to poor pharmacokinetic properties, low binding affinity, and poor in vivo stability. The bioactive conformation, however, has been reported. In the bound conformation, studies show the carboxylate of NeuNAc forms a salt bridge with an arginine of the receptor and the GlcNAc acts as a spacer stacking the Fuc and Gal carbohydrate moieties upon each other in the appropriate spatial orientation. Modifications that disrupt the bioactive conformation have been shown to result in a loss of activity.

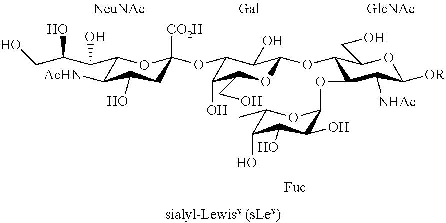

Other modulators of selectin-mediated function include the PSGL-1 protein (and smaller peptide fragments), fucoidan, glycyrrhizin (and derivatives), anti-selectin antibodies, sulfated lactose derivatives, and heparin. All have shown to be unsuitable for drug development due to insufficient activity, toxicity, lack of specificity, poor pharmacokinetic properties and/or availability of material.

### SUMMARY OF THE INVENTION

The present application discloses compounds and pharmaceutical compositions comprising at least one such compound that may be useful for treating and/or preventing diseases, disorders, and/or conditions that are associated with E-selectin activity including, for example, inflammatory diseases and cancer.

In some embodiments, the present disclosure is directed to compounds of Formula (I):

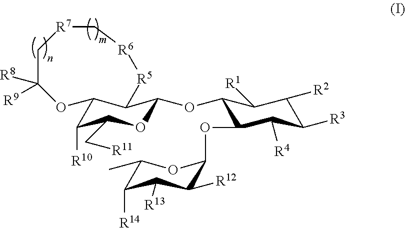

wherein

R¹ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;

R² is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, -M, -L-M, —C(═O)OY¹, and —C(═O)NY¹Y² groups, wherein Y¹ and Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y¹ and Y² may join together to form a ring;

R³ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, -M, -L-M, —C(═O)OY³, and —C(═O)NY³Y⁴ groups, wherein Y³ and Y⁴, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y³ and Y⁴ may join together to form a ring;

R⁴ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;

R⁵ is chosen from O, S, and NR¹⁵;

R⁶ is chosen from a bond, C(═0), and CR¹⁶R¹⁷;

R⁷ is chosen from C₂₋₈ alkylene, C₂₋₁₂ heterocyclyl, C₆₋₁₈ aryl, C₂₋₁₃ heteroaryl, and CR¹⁸R¹⁹ groups;

R⁸ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₁₋₈ alkoxy, C₆₋₁₈ aryl, and C₂₋₁₃ heteroaryl groups, or R⁸ joins together with R⁹ to form a ring;

R⁹ is chosen from —Z, —CH₂OH, —CH₂OY⁵, —OH, —OY⁵, —CN, —C(═O)Y⁵, —C(═O)OH, —C(═O)OY⁵, —C(═O)NY⁵Y⁶, —S(═O)₂Y⁵, —S(═O)₂OY⁵, and —S(═O)₂NY⁵Y⁶ groups, wherein Y⁵ and Y⁶, which may be identical or different, are independently chosen from C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y⁵ and Y⁶ may join together to form a ring, or R⁹ joins together with R⁸ or R¹⁸ to form a ring;

R¹⁰ is chosen from H, —OH, F, Cl, Br, —CF₂H, and —NY⁷Y⁸, wherein Y⁷ and Y⁸, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y⁷ and Y⁸ may join together to form a ring;

R¹¹ is chosen from H, —OH, F, Cl, Br, —CF₂H, and —NY⁹Y¹⁰, wherein Y⁹ and Y¹⁰, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y⁹ and Y¹⁰ may join together to form a ring;

R¹² is chosen from —OH, F, Cl, Br, —CF₂H, and —NY¹¹Y¹², wherein Y¹¹ and Y¹², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y¹¹ and Y¹² may join together to form a ring;

R¹³ is chosen from —OH, F, Cl, Br, —CF₂H, and —NY¹³Y¹⁴, wherein Y¹³ and Y¹⁴, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y¹³ and Y¹⁴ may join together to form a ring;

R¹⁴ is chosen from —OH, F, Cl, Br, —CF₂H, and —NY⁷Y⁸, wherein Y¹⁵ and Y¹⁶, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y¹⁵ and Y¹⁶ may join together to form a ring;

R¹⁵ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;

R¹⁶ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, or R¹⁶ joins together with R¹⁷ to form a ring;

R¹⁷ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, or R¹⁷ joins together with R¹⁶ to form a ring;

R¹⁸ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₁₋₈ alkoxy groups, or R¹⁸ joins together with R⁹ or R¹⁹ to form a ring;

R¹⁹ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₁₋₈ alkoxy groups, or R¹⁹ joins together with R¹⁸ to form a ring;

L is chosen from linker groups;

M is chosen from non-glycomimetic moieties;

Z is chosen from acid bioisosteric moieties;

m is chosen from integers ranging from 0 to 5; and

n is chosen from integers ranging from 0 to 5.

As used herein, ‘compound of Formula (I)’ includes E-selectin antagonists of Formula (I), pharmaceutically acceptable salts of E-selectin antagonists of Formula (I), prodrugs of E-selectin antagonists of Formula (I), and pharmaceutically acceptable salts of prodrugs of E-selectin antagonists of Formula (I).

In some embodiments, the present disclosure is directed to pharmaceutical compositions comprising at least one compound of Formula (I) and optionally at least one pharmaceutically acceptable ingredient.

In some embodiments, the present disclosure is directed to a method for treatment and/or prevention of at least one disease, disorder, and/or condition where inhibition of E-selectin mediated functions is useful comprising administering to a subject in need thereof an effective amount of at least one compound of Formula (I) or a pharmaceutical composition comprising at least one compound of Formula (I) and optionally at least once pharmaceutically acceptable ingredient.

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

### BRIEF DESCRIPTION OF THE DRAWINGS

FIG. 1 (FIG. 1A and FIG. 1B) is a diagram illustrating the synthesis of an embodiment (compounds 18a and 18b) of the glycomimetic compounds disclosed herein.

FIG. 2 is a diagram illustrating the synthesis of an embodiment (compound 25) of the glycomimetic compounds disclosed herein.

FIG. 3 is a diagram illustrating the synthesis of an embodiment (compounds 26) of the glycomimetic compounds disclosed herein.

FIG. 4 (FIG. 4A and FIG. 4B) is a diagram illustrating the synthesis of an embodiment (compounds 39) of the glycomimetic compounds disclosed herein.

FIG. 5 is a diagram illustrating the synthesis of an embodiment (compounds 47 and 48) of the glycomimetic compounds disclosed herein.

FIG. 6 (FIG. 6A and FIG. 6B) is a diagram illustrating the synthesis of an embodiment (compounds 60 and 61) of the glycomimetic compounds disclosed herein.

### DETAILED DESCRIPTION

Disclosed herein are glycomimetic E-selectin antagonists, pharmaceutical compositions comprising the same, and methods for inhibiting E-selectin-mediated functions using the same. The compounds and compositions of the present disclosure may be useful for treating and/or preventing (i.e., reducing the likelihood of occurrence or recurrence of) diseases, disorders, and/or conditions that are treatable by inhibiting binding of E-selectin to one or more E-selectin ligands.

The compounds of the present disclosure include trisaccharide domain mimics in which the naturally occurring NeuNAc monomer has been replaced by at least one other anionic moiety (sialic acid mimic) and/or wherein the carboxyl or carbonyl of the sialic acid mimic is positioned in its bioactive conformation by a macrocycle created through the 2^{nd} and 3^{rd} positions on the galactose. Further modifications of the hydroxyl and carboxyl groups are also possible.

The compounds of the present disclosure may result in a reduced molecular weight, polar surface area, and/or the number of hydrogen bond donors and acceptors as compared to the tetrasaccharide epitope sLe^{x}, and, as a consequence, the compounds of the present disclosure may have at least one improved physicochemical, pharmacological and/or pharmacokinetic property.

In some embodiments, the present disclosure is directed to at least one compound chosen from compounds of Formula (I):

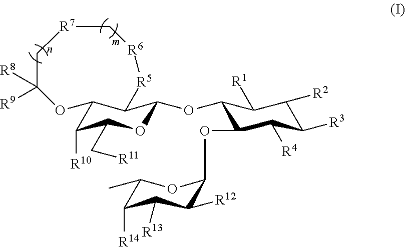

wherein

R¹ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;

R² is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, -M, -L-M, —C(═O)OY¹, and —C(═O)NY¹Y² groups, wherein Y¹ and Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y¹ and Y² may join together to form a ring;

R³ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, -M, -L-M, —C(═O)OY³, and —C(═O)NY³Y⁴ groups, wherein Y³ and Y⁴, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y³ and Y⁴ may join together to form a ring;

R⁴ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;

R⁵ is chosen from 0, S, and NR¹⁵;

R⁶ is chosen from a bond, C(═O), and CR¹⁶R¹⁷;

R⁷ is chosen from C₂₋₈ alkylene, C₂₋₈ alkenyl, C₂₋₈ alkynyl, C₂₋₁₂ heterocyclyl, C₆₋₁₈ aryl, C₂₋₁₃ heteroaryl, and CR¹⁸R¹⁹ groups;

R⁸ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₁₋₈ alkoxy, C₆₋₁₈ aryl, and C₂₋₁₃ heteroaryl groups, or R⁸ joins together with R⁹ to form a ring;

R⁹ is chosen from —Z, —CH₂OH, —CH₂OY⁵, —OH, —OY⁵, —CN, —C(═O)Y⁵, —C(═O)OH, C(═O)OY⁵, —C(═O)NY⁵Y⁶, —S(═O)₂Y⁵, —S(═O)₂OY⁵, and —S(═O)₂NY⁵Y⁶ groups, wherein Y⁵ and Y⁶, which may be identical or different, are independently chosen from C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y⁵ and Y⁶ may join together to form a ring, or R⁹ joins together with R⁸ or R¹⁸ to form a ring;

R¹⁰ is chosen from H, —OH, F, Cl, Br, —CF₂H, and —NY⁷Y⁸, wherein Y⁷ and Y⁸, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y⁷ and Y⁸ may join together to form a ring;

R¹¹ is chosen from H, —OH, F, Cl, Br, —CF₂H, and —NY⁹Y¹⁰, wherein Y⁹ and Y¹⁰, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y⁹ and Y¹⁰ may join together to form a ring;

R¹² is chosen from —OH, F, Cl, Br, —CF₂H, and —NY¹¹Y¹², wherein Y¹¹ and Y¹², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y¹¹ and Y¹² may join together to form a ring;

R¹³ is chosen from —OH, F, Cl, Br, —CF₂H, and —NY¹³Y¹⁴, wherein Y¹³ and Y¹⁴, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y¹³ and Y¹⁴ may join together to form a ring;

R¹⁴ is chosen from —OH, F, Cl, Br, —CF₂H, and —NY⁷Y⁸, wherein Y¹⁵ and Y¹⁶, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y¹⁵ and Y¹⁶ may join together to form a ring;

R¹⁵ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;

R¹⁶ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, or R¹⁶ joins together with R¹⁷ to form a ring;

R¹⁷ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, or R¹⁷ joins together with R¹⁶ to form a ring;

R¹⁸ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₁₋₈ alkoxy groups, or R¹⁸ joins together with R⁹ or R¹⁹ to form a ring;

R¹⁹ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₁₋₈ alkoxy groups, or R¹⁹ joins together with R¹⁸ to form a ring;

L is chosen from linker groups;

M is chosen from non-glycomimetic moieties;

Z is chosen from acid bioisosteric moieties;

m is chosen from integers ranging from 0 to 5; and

n is chosen from integers ranging from 0 to 5.

In some embodiments, m and n are chosen such that the sum of m and n is an integer ranging from 0 to 5. In some embodiments, m and n are chosen such that the sum of m and n is an integer ranging from 0 to 6. In some embodiments, m and n are chosen such that the sum of m and n is 2. In some embodiments, m and n are chosen such that the sum of m and n is 1.

In some embodiments, R¹ is chosen from H, C₁₋₄ alkyl, and C₁₋₄ haloalkyl groups. In some embodiments, R¹ is chosen from H, methyl, ethyl, —CH₂F, —CHF₂, —CF₃, —CH₂CH₂F, —CH₂CHF₂, and CH₂CF₃. In some embodiments R¹ is H. In some embodiments, R′ is chosen from methyl and ethyl. In some embodiments, R¹ is chosen from methyl. In some embodiments, R¹ is chosen from ethyl.

In some embodiments, R² is chosen from H, —C(═O)OY¹, and —C(═O)NY¹Y². In some embodiments, R³ is chosen from H, —C(═O)OY¹, and —C(═O)NY¹Y². In some embodiments, R² and/or R³ is H. In some embodiments, R¹, R², and R³ are each H.

In some embodiments, R⁴ is chosen from H, C₁₋₄ alkyl, and C₁₋₄ haloalkyl groups. In some embodiments, R⁴ is chosen from H, methyl, ethyl, —CH₂F, —CHF₂, —CF₃, —CH₂CH₂F, —CH₂CHF₂, and —CH₂CF₃. In some embodiments R⁴ is H. In some embodiments, R⁴ is chosen from methyl and ethyl. In some embodiments, R⁴ is chosen from methyl. In some embodiments, R⁴ is chosen from ethyl. In some embodiments, R¹ is H and R⁴ is chosen from methyl and ethyl.

In some embodiments, R⁵ is O. In some embodiments, R⁵ is NR¹⁵. In some embodiments, R¹⁵ is chosen from H, C₁₋₈ alkyl, and C₁₋₈ haloalkyl groups.

In some embodiments, R⁶ is a bond. In some embodiments, R⁶ is chosen from C(═O) and CR¹⁶R¹⁷ groups. In some embodiments, R⁶ is chosen from CR¹⁶R¹⁷ groups. In some embodiments, R¹⁶ is chosen from H and C₁₋₈ alkyl groups. In some embodiments, R¹⁷ is chosen from H and C₁₋₈ alkyl groups. In some embodiments, R¹⁶ and/or R¹⁷ is H. In some embodiments, R¹⁶ and R¹⁷ are each H. In some embodiments, R¹⁶ joins together with R¹⁷ to form a ring.

In some embodiments, the at least one compound is chosen from compounds of Formula (Ia):

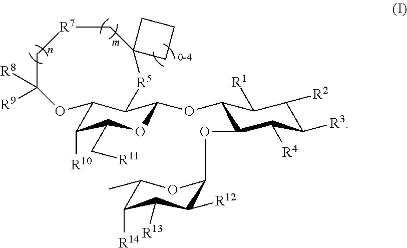

In some embodiments, the at least one compound is chosen from compounds of the following Formulas:

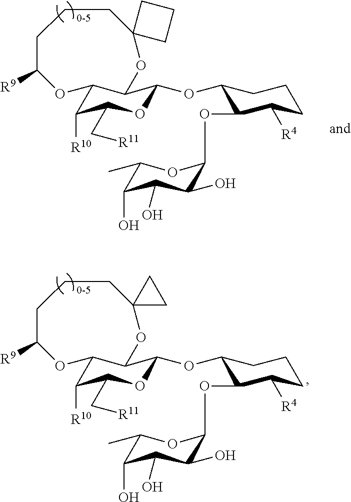

wherein

R⁴ is chosen from H, C₁₋₄ alkyl, and C₁₋₄ haloalkyl groups;

R⁹ is chosen from —Z, —C(═O)OH, —C(═O)OY⁵, and —C(═O)NY⁵Y⁶;

R¹⁰ is chosen from H, —OH, F, and —CF₂H; and

R¹¹ is chosen from H, —OH, and —CF₂H.

In some embodiments, R⁷ is chosen from CR¹⁸R¹⁹ groups. In some embodiments, R¹⁸ is chosen from H and C₁₋₈ alkyl groups. In some embodiments, R¹⁹ is chosen from H and C₁₋₈ alkyl groups. In some embodiments, R¹⁸ is H. In some embodiments, R¹⁹ is H. In some embodiments, R¹⁸ and R¹⁹ are each H. In some embodiments, R¹⁸ joins together with R¹⁹ to form a ring.

In some embodiments, the at least one compound is chosen from compounds of Formula (Ib):

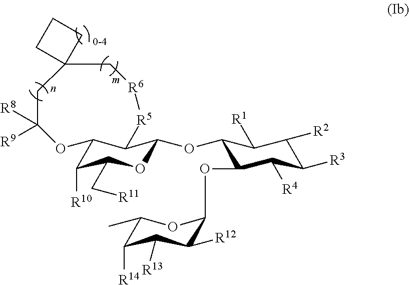

In some embodiments, the at least one compound is chosen from compounds of the following Formulas:

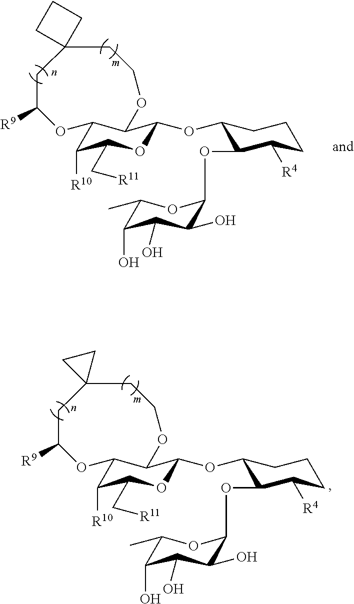

wherein

R⁴ is chosen from H, C₁ alkyl, and C₁₋₄ haloalkyl groups;

R⁹ is chosen from —Z, —C(═O)OH, —C(═O)OY⁵, and —C(═O)NY⁵Y⁶;

R¹⁰ is chosen from H, —OH, F, and —CF₂H;

R¹¹ is chosen from H, —OH, and —CF₂H; and

m and n are chosen such that the sum of m and n is an integer ranging from 0 to 6.

In some embodiments, the at least one compound is chosen from compounds of Formula (Ic):

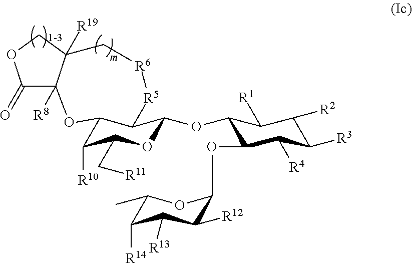

In some embodiments, the at least one compound is chosen from compounds of the following Formula:

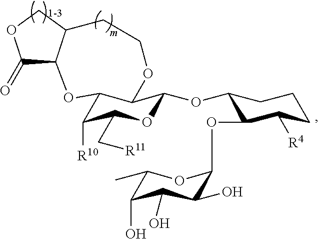

wherein

R⁴ is chosen from H, C₁₋₄ alkyl, and C₁₋₇ haloalkyl groups;

R¹⁰ is chosen from H, —OH, F, and —CF₂H; and

R¹¹ is chosen from H, —OH, and —CF₂H.

In some embodiments, the at least one compound is chosen from compounds of Formulas (Id), (Ie), and (If):

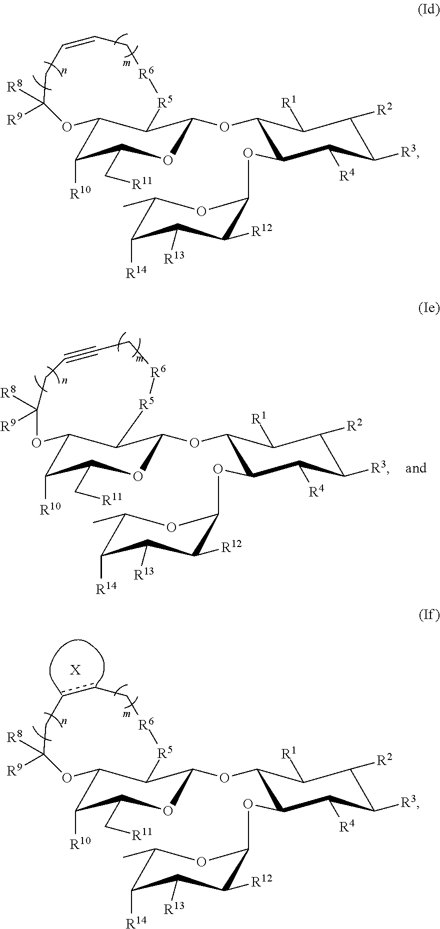

wherein X represents a carbocyclic, heterocyclic, aromatic, or heteroaromatic ring.

In some embodiments, the at least one compound is chosen from compounds of the following Formula:

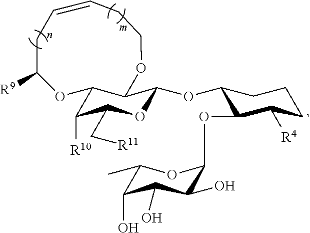

wherein

R⁴ is chosen from H, C₁₋₄ alkyl, and C₁₋₄ haloalkyl groups;

R⁹ is chosen from —Z, —C(═O)OH, —C(═O)OY⁵, and —C(═O)NY⁵Y⁶;

R¹⁰ is chosen from H, —OH, F, and —CF₂H;

R¹¹ is chosen from H, —OH, and —CF₂H; and

m and n are chosen such that the sum of m and n is an integer ranging from 0 to 5.

In some embodiments, the at least one compound is chosen from compounds of the following Formula:

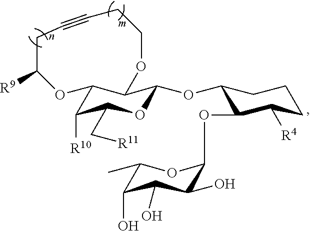

wherein

R⁴ is chosen from H, C₁₋₄ alkyl, and C₁₋₄ haloalkyl groups;

R⁹ is chosen from —Z, —C(═O)OH, —C(═O)OY⁵, and —C(═O)NY⁵Y⁶;

R¹⁰ is chosen from H, —OH, F, and —CF₂H;

R¹¹ is chosen from H, —OH, and —CF₂H; and

m and n are chosen such that the sum of m and n is an integer ranging from 0 to 5.

In some embodiments, the at least one compound is chosen from compounds of the following Formulas:

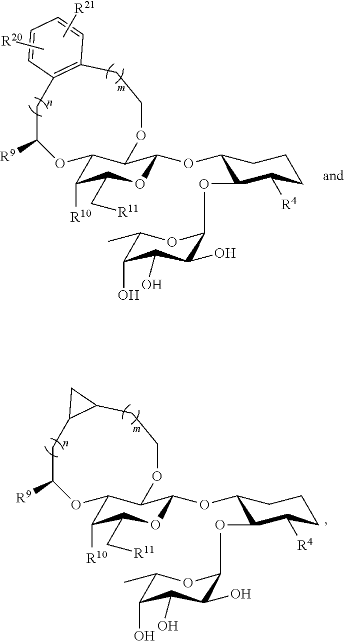

wherein

R⁴ is chosen from H, C₁₋₄ alkyl, and C₁₋₄ haloalkyl;

R⁹ is chosen from —Z, —C(═O)OH, —C(═O)OY⁵, and —C(═O)NY⁵Y⁶;

R¹⁰ is chosen from H, —OH, F, and —CF₂H;

R¹¹ is chosen from H, —OH, and —CF₂H;

R²⁰ is chosen from H, halo, C₁₋₆ alkyl, C₁₋₆ haloalkyl, —OH, —O—C₁₋₆ alkyl, C₂₋₆ heterocyclyl, C₆₋₁₀ aryl, C₂₋₈ heteroaryl, and —C(═O)OY¹⁷ groups, wherein Y¹⁷ is chosen from H, C₁₋₆ alkyl, C₂₋₁₂ heterocyclyl, C₆₋₁₀ aryl, and C₂₋₈ heteroaryl groups, or R²⁰ joins together with R²¹ to form a ring;

R²¹ is chosen from H, halo, C₁₋₆ alkyl, C₁₋₆ haloalkyl, OH, O—C₁₋₆ alkyl, C₂₋₆ heterocyclyl, C₆₋₁₀ aryl, C₂₋₈ heteroaryl, and —C(═O)OY¹⁸ groups, wherein Y¹⁸ is chosen from H, C₁₋₆ alkyl, C₂₋₁₂ heterocyclyl, C₆₋₁₀ aryl, and C₂₋₈ heteroaryl groups, or R²¹ joins together with R²⁰ to form a ring; and
m and n are chosen such that the sum of m and n is an integer ranging from 0 to 5.

In some embodiments, R²⁰ and/or R²¹ is H. In some embodiments, R²⁰ and R²¹ are each H. In some embodiments, R²⁰ is chosen from halo. In some embodiments, R²¹ is chosen from halo. In some embodiments, R²⁰ and/or R²¹ is F. In some embodiments, R²⁰ and/or R²¹ is OH. In some embodiments, R²⁰ and/or R²¹ is OMe. In some embodiments, R²⁰ and R²¹ join together to form a ring.

In some embodiments, R⁸ is chosen from H, C₁₋₄ alkyl, C₆₋₁₈ aryl, and C₂₋₁₃ heteroaryl groups. In some embodiments, R⁸ is H.

In some embodiments, R⁹ is chosen from —Z, —C(═O)OH, C(═O)OY⁵, and C(═O)NY⁵Y⁶. In some embodiments, R⁹ is Z. In some embodiments, R⁹ is C(═O)OH. In some embodiments, R⁸ joins together with R⁹ to form a ring.

In some embodiments, the at least one compound is chosen from compounds of Formulas (Ig) and (Ih):

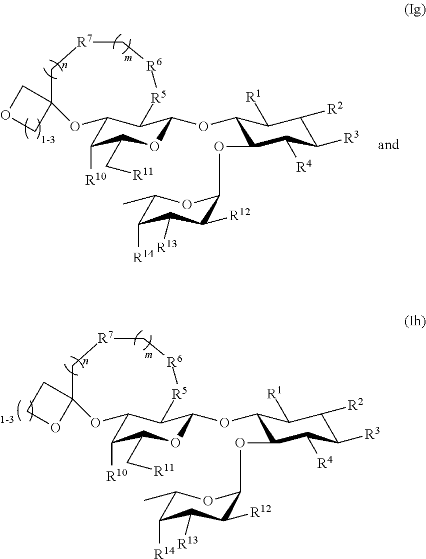

In some embodiments, the at least one compound is chosen from compounds of the following Formulas:

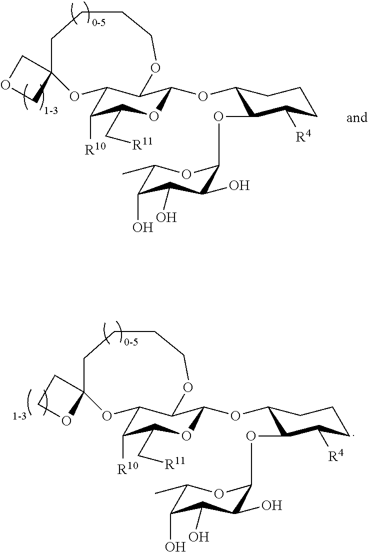

In some embodiments, R¹⁰ is chosen from H, —OH, F, and —CF₂H. In some embodiments, R¹⁰ is —OH. In some embodiments, R¹⁰ is F. In some embodiments, R¹¹ is chosen from H, —OH, and —CF₂H. In some embodiments, R¹¹ is OH. In some embodiments, R¹⁰ and R¹¹ are each —OH.

In some embodiments, R¹², R¹³, and/or R¹⁴ is OH. In some embodiments, R¹², R¹³, and R¹⁴ are each —OH.

In some embodiments, linker groups may be chosen from (or may include) spacer groups, such as, for example, —(CH₂)ₚ— and —O(CH₂)ₚ—, wherein p is chosen from integers ranging from 1 to 20. Other non-limiting examples of spacer groups include carbonyl groups and carbonyl-containing groups such as, for example, amide groups. A non-limiting example of a spacer group is

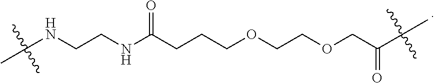 [SMILES: *NCCNC(=O)CCCOCCOCC(*)=O]

In some embodiments, the linker group is chosen from

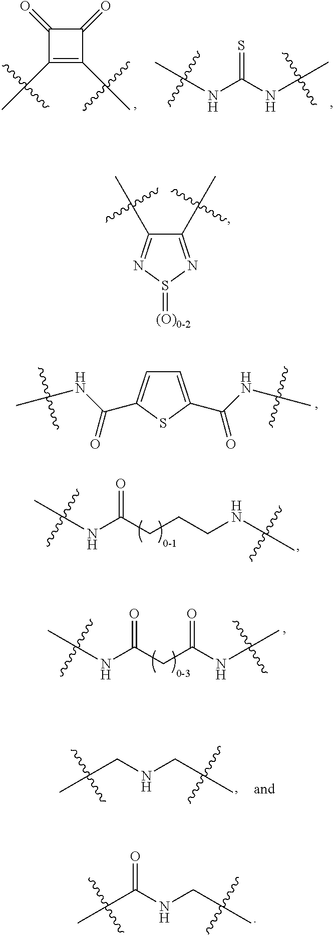

Other linker groups, such as, for example, polyethylene glycols (PEGS) and —C(═O)—NH—(CH₂)ₚ—C(═O)—NH—, wherein p is chosen from integers ranging from 1 to 20, will be familiar to those of ordinary skill in the art and/or those in possession of the present disclosure.

In some embodiments, the linker group is

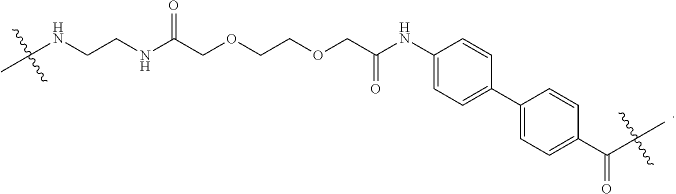 [SMILES: *NCCNC(=O)COCCOCC(=O)Nc1ccc(-c2ccc(C(*)=O)cc2)cc1]

In some embodiments, the linker group is

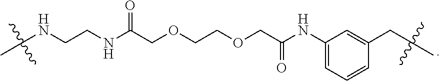 [SMILES: *Cc1cccc(NC(=O)COCCOCC(=O)NCCN*)c1]

In some embodiments, the non-glycomimetic moieties are chosen from polyethylene glycol, thiazolyl, chromenyl, —C(═O)NH(CH₂)₁₋₄NH₂, C₁₋₈ alkyl, and C(O)OY¹⁹, wherein Y¹⁹ is chosen from C₁₋₄ alkyl, C₂₋₄ alkenyl, and C₂₋₄ alkynyl groups.

Acid bioisosteric moieties may be chosen from those well-known by persons of skill in the art. See, e.g., Ballatore et al., ChemMedChem 8:385-395 (2013) and Meanwell, J. Med. Chem. 54:2529-91 (2011). In some embodiments, the acid bioisosteric moieties are chosen from

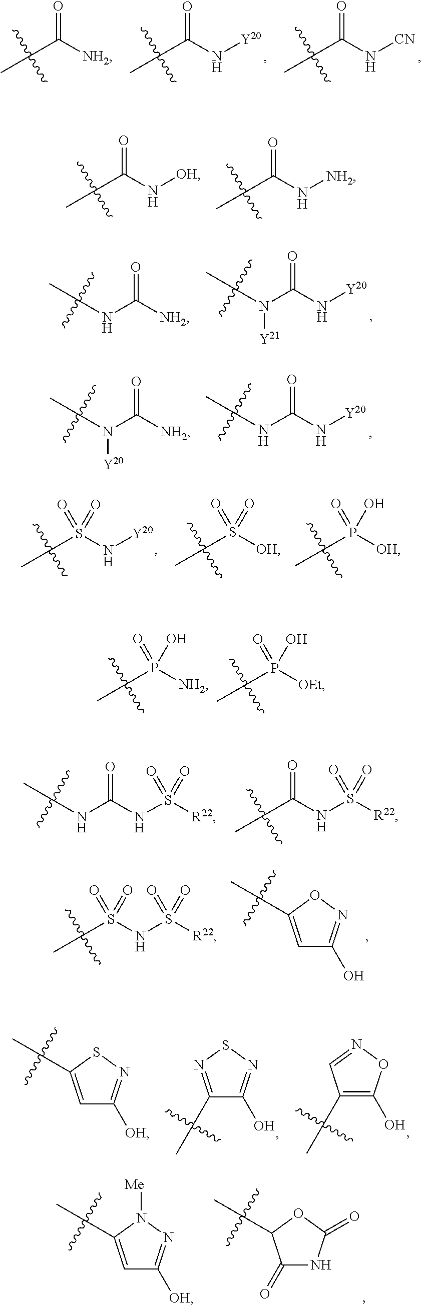

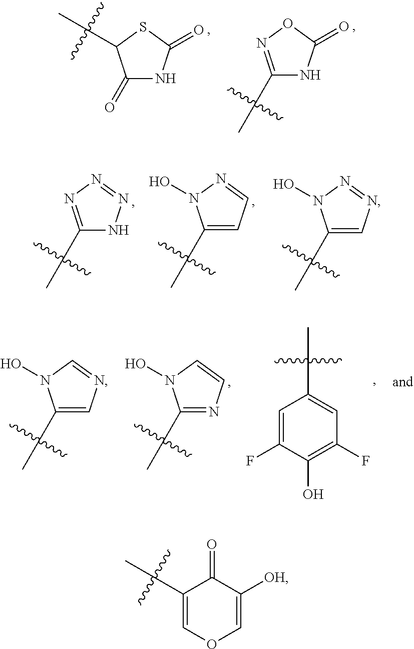

wherein
R²² is chosen from C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₂₋₁₂ heterocyclyl, C₆₋₁₀ aryl, and C₂₋₈ heteroaryl groups;
Y²⁰ is chosen from C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₂₋₁₂ heterocyclyl, C₆₋₁₀ aryl, and C₂₋₈ heteroaryl groups; and
Y²¹ is chosen from C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₂₋₁₂ heterocyclyl, C₆₋₁₀ aryl, and C₂₋₈ heteroaryl groups.

In some embodiments, R²² is chosen from C₁₋₁₂ alkyl, C₂₋₁₂ heterocyclyl, C₆₋₁₀ aryl, and C₂₋₈ heteroaryl groups. In some embodiments, R²² is chosen from C₁₋₆ alkyl groups. In some embodiments, R²² is chosen from methyl, ethyl, propyl, and butyl groups.

In some embodiments, Y²⁰ is chosen from C₁₋₁₂ alkyl, C₂₋₁₂ heterocyclyl, C₆₋₁₀ aryl, and C₂₋₈ heteroaryl groups. In some embodiments, Y²⁰ is chosen from C₁₋₁₂ alkyl, C₆₋₁₀ aryl, and C₂₋₈ heteroaryl groups. In some embodiments, Y²⁰ is chosen from C₁₋₆ alkyl groups. In some embodiments, Y²⁰ is chosen from methyl, ethyl, propyl, and butyl groups.

In some embodiments, Y²¹ is chosen from C₁₋₁₂ alkyl, C₂₋₁₂ heterocyclyl, C₆₋₁₀ aryl, and C₂₋₈ heteroaryl groups. In some embodiments, Y²¹ is chosen from C₁₋₁₂ alkyl, C₆₋₁₀ aryl, and C₂₋₈ heteroaryl groups. In some embodiments, Y²¹ is chosen from C₁₋₆ alkyl groups. In some embodiments, Y²¹ is chosen from methyl, ethyl, propyl, and butyl groups.

In some embodiments, the at least one compound is chosen from compounds of the following Formulas:

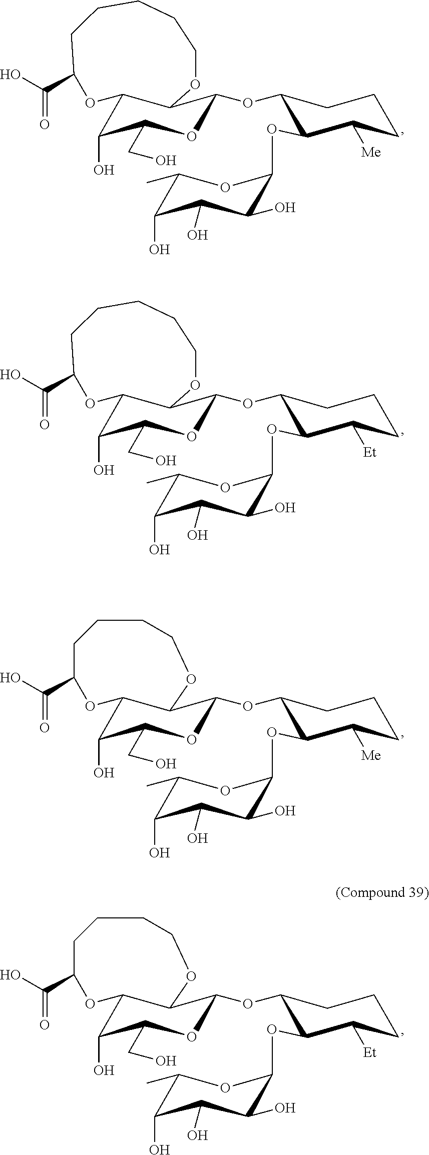

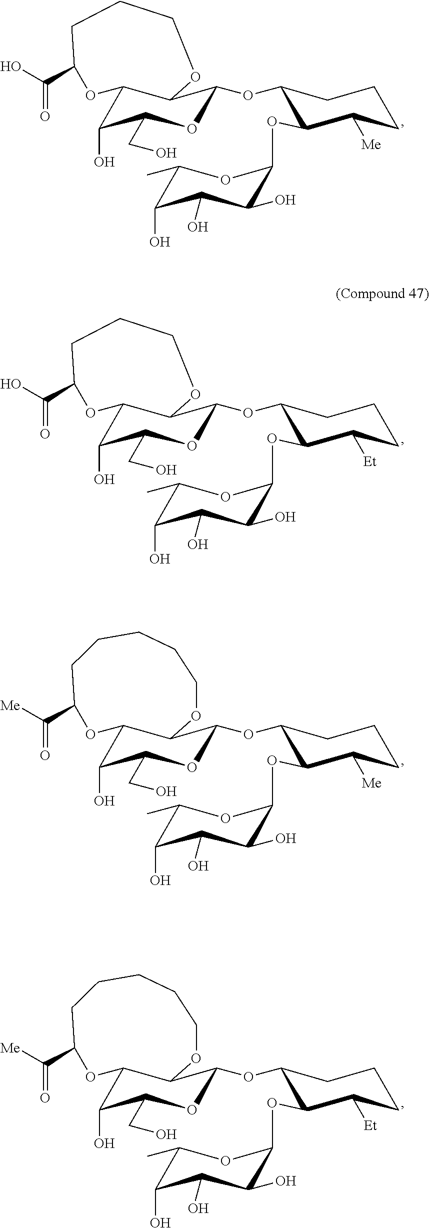

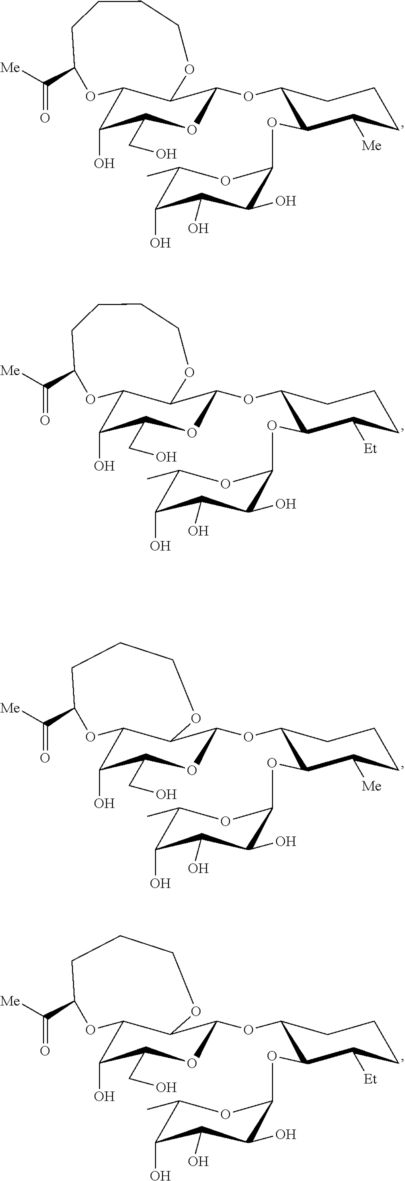

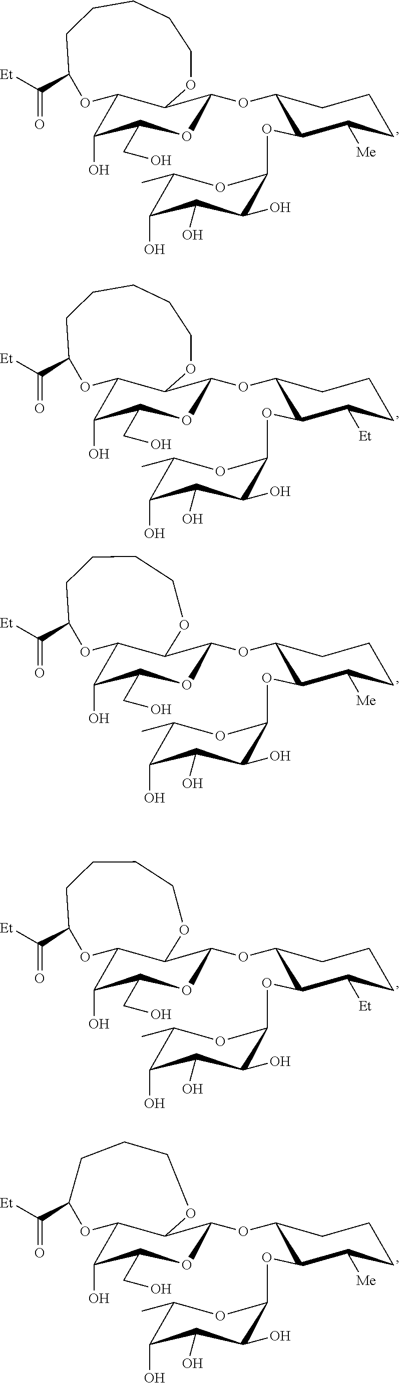

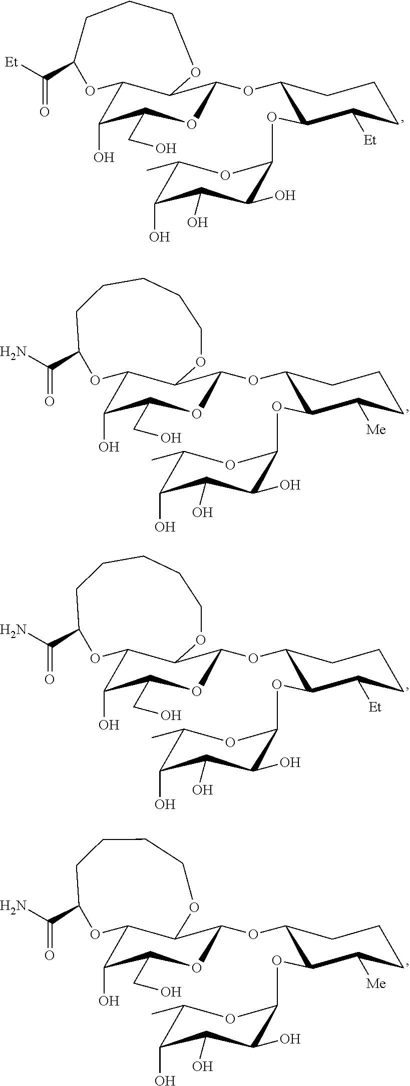

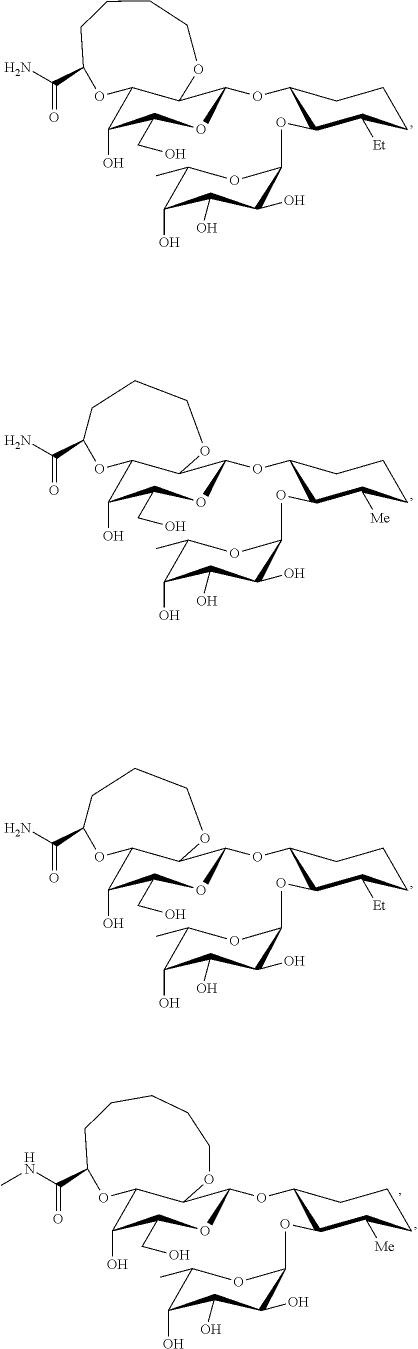

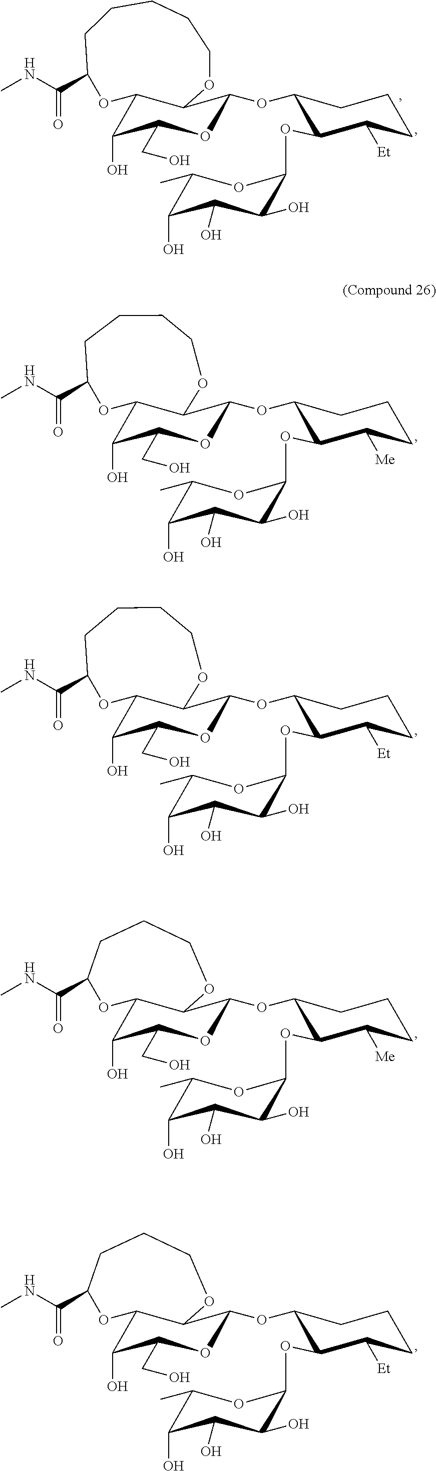

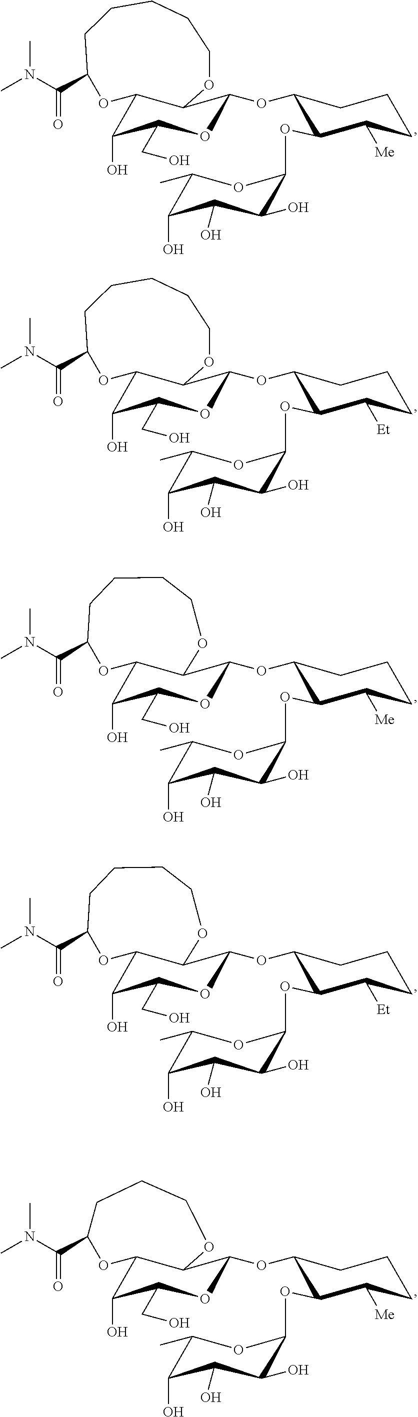

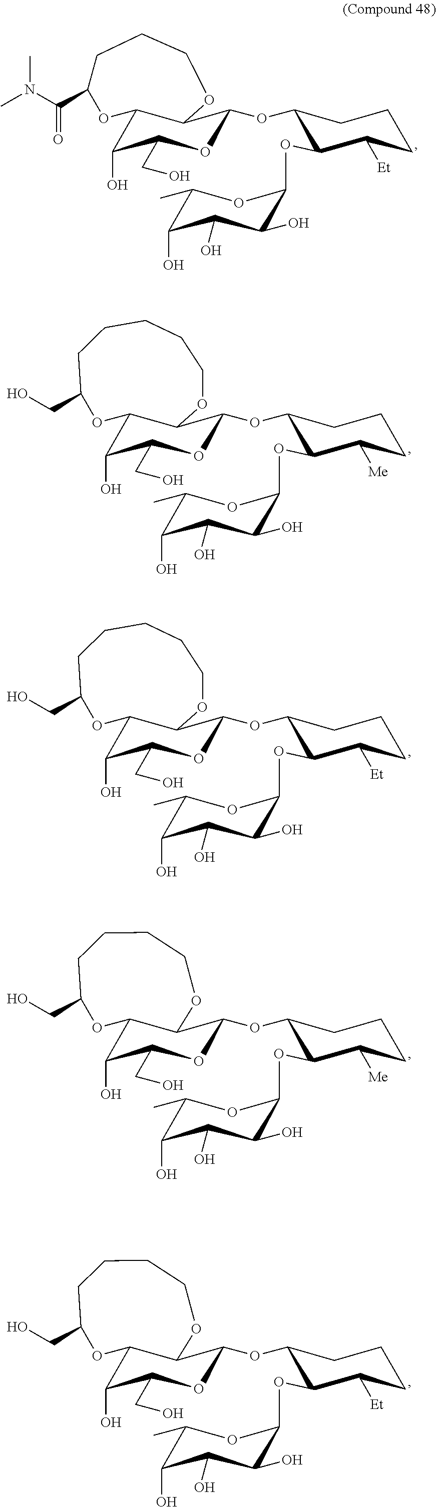

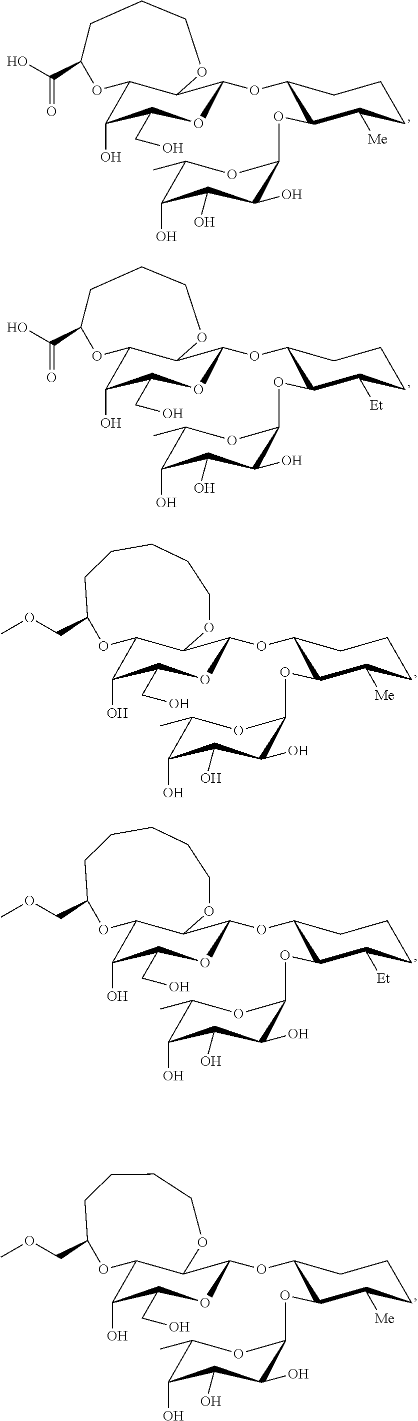

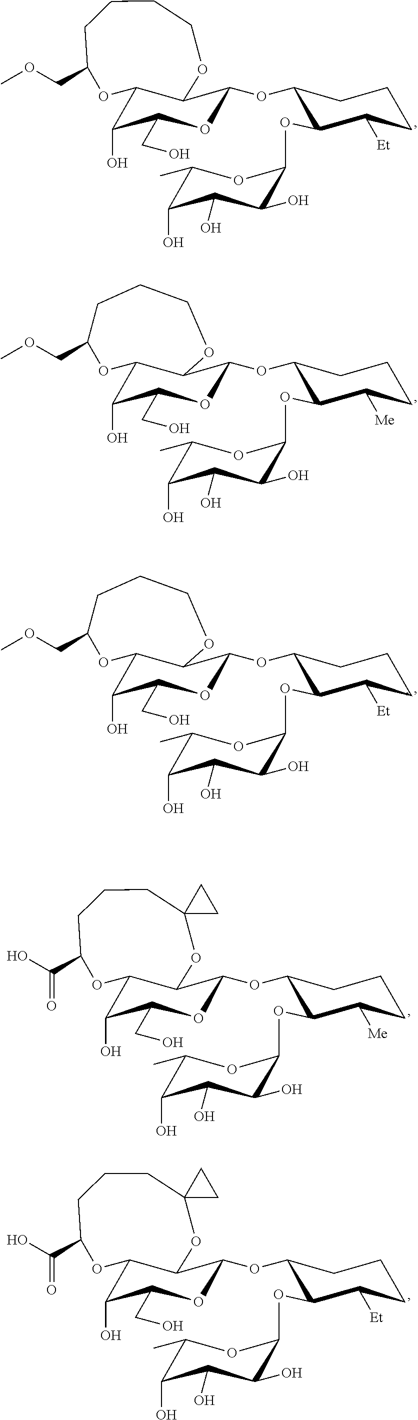

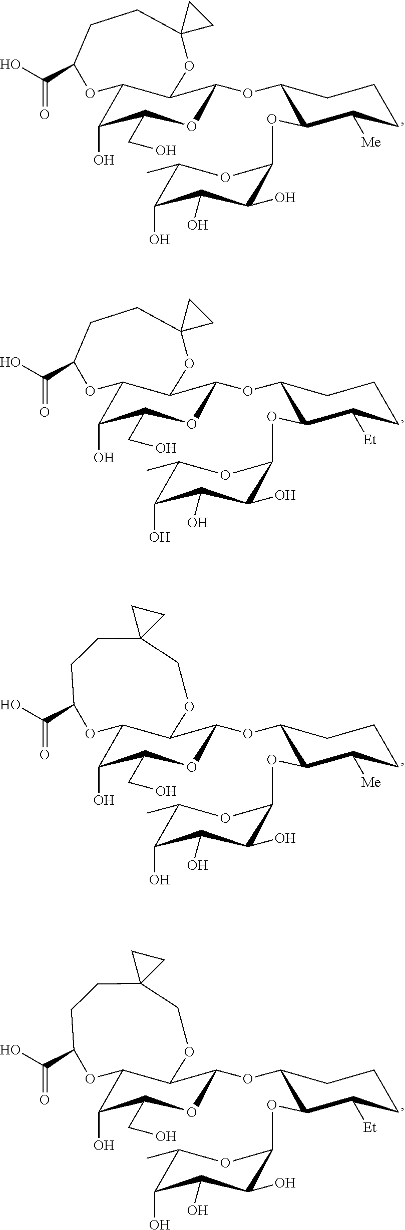

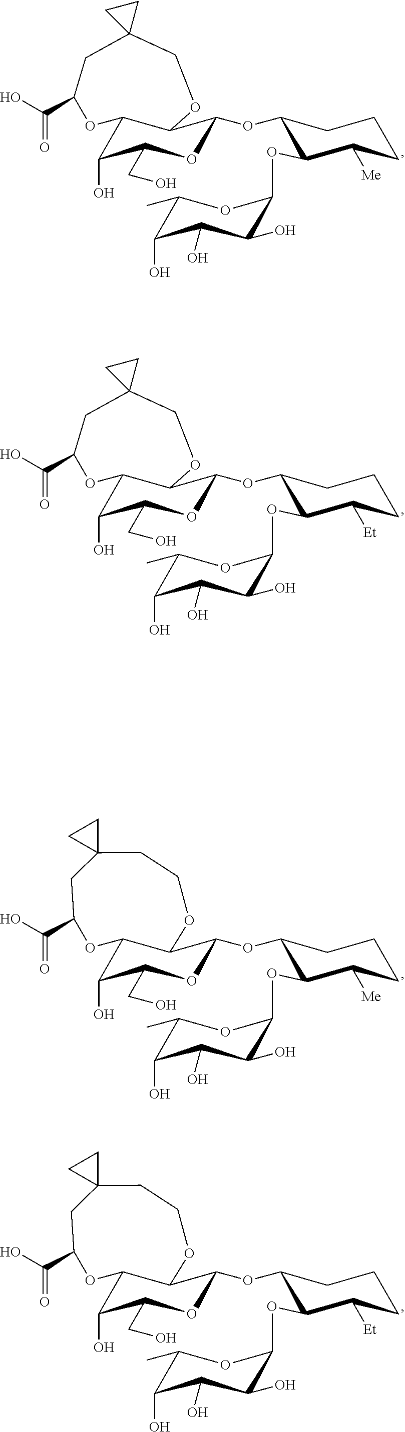

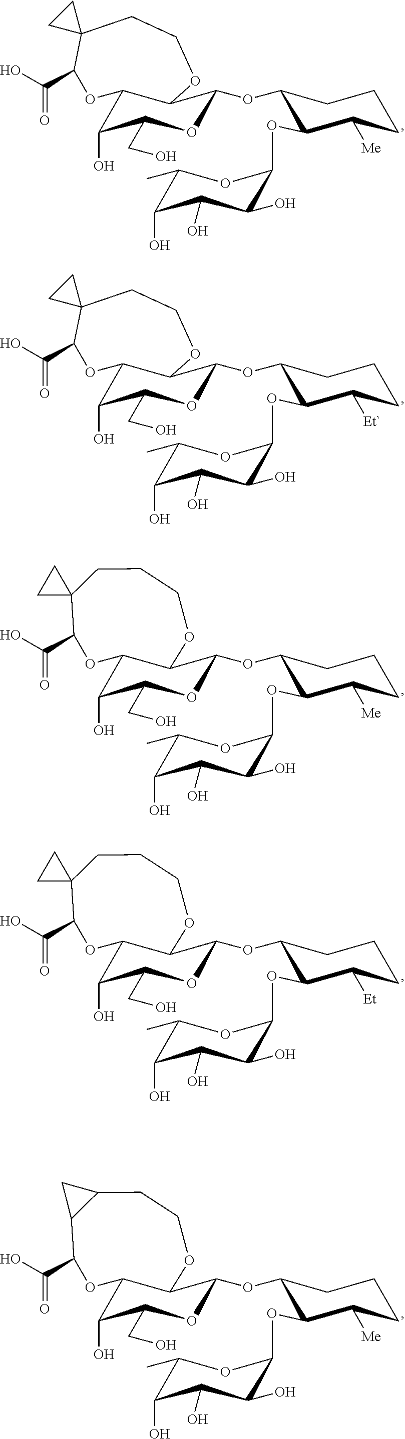

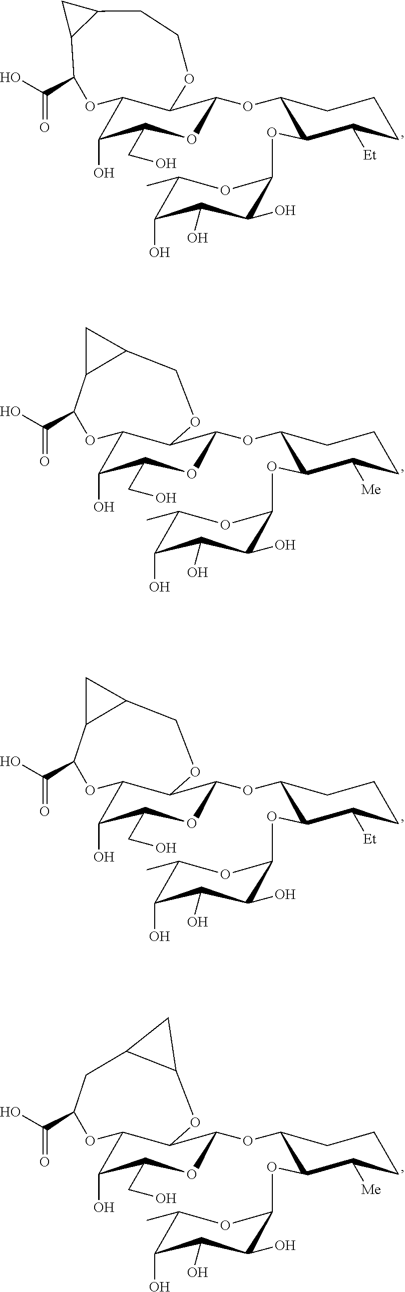

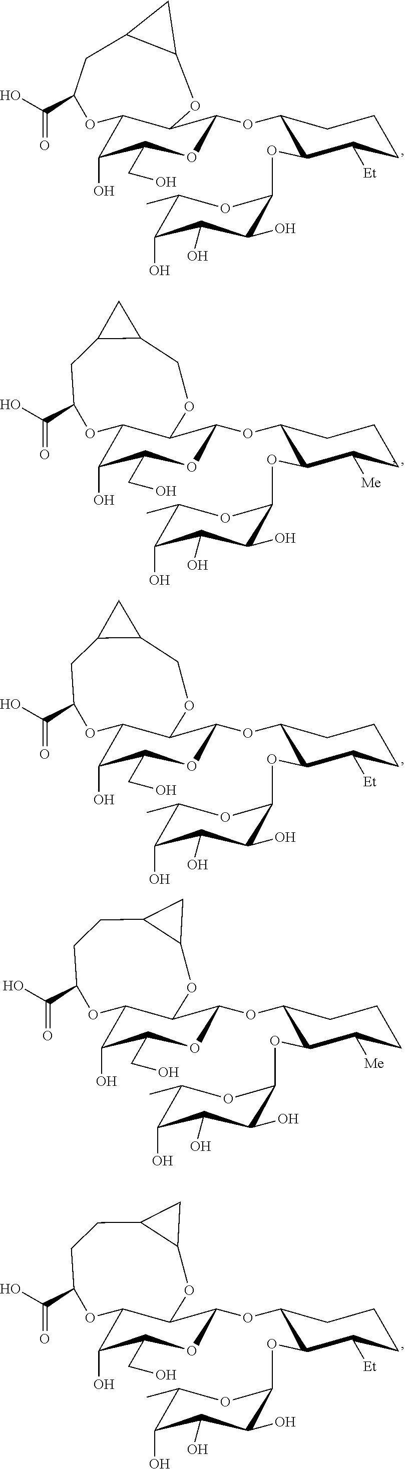

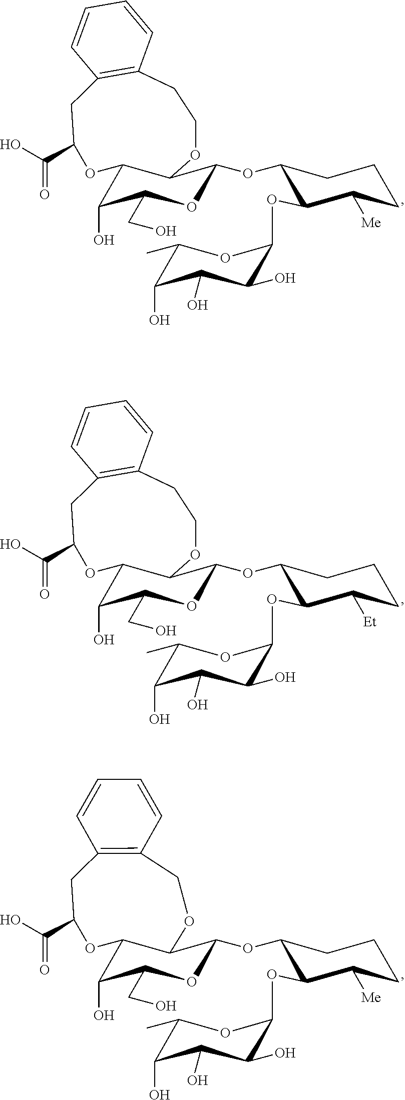

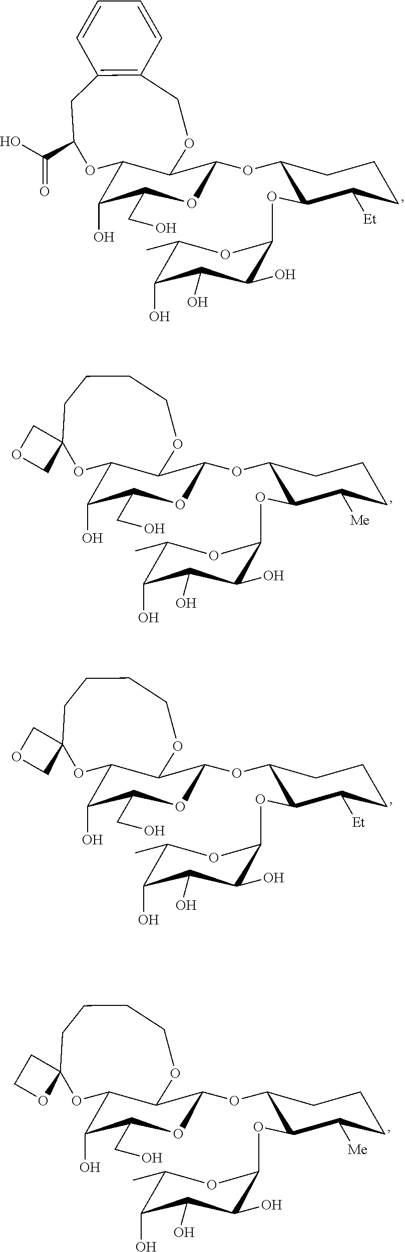

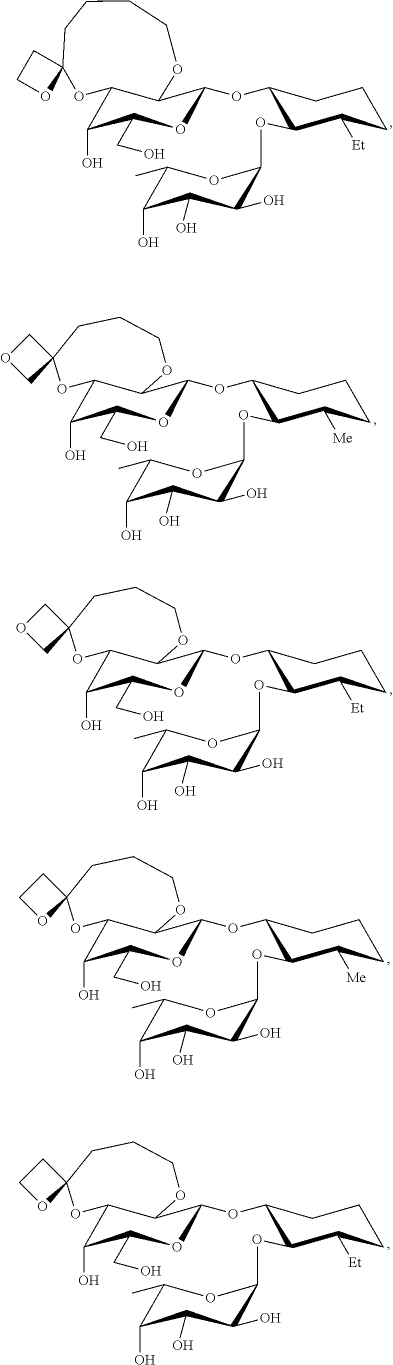

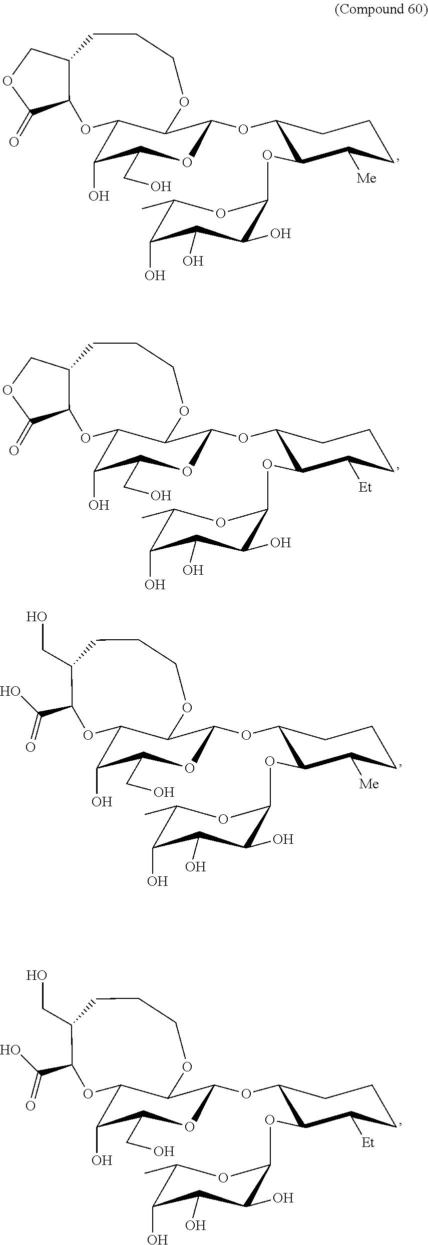

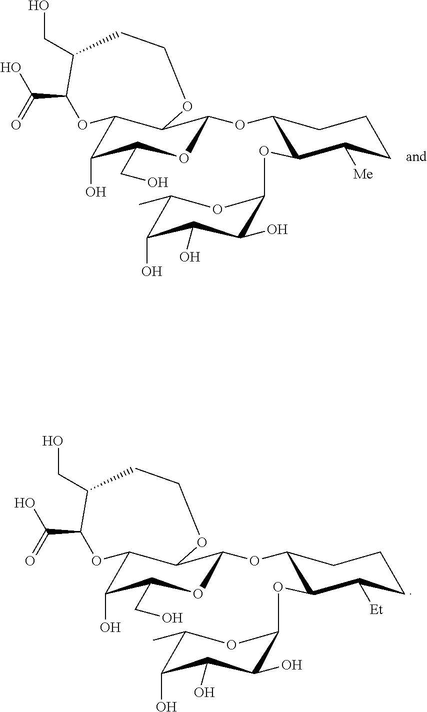

In some embodiments, the at least one compound is chosen from compounds of the following Formulas:

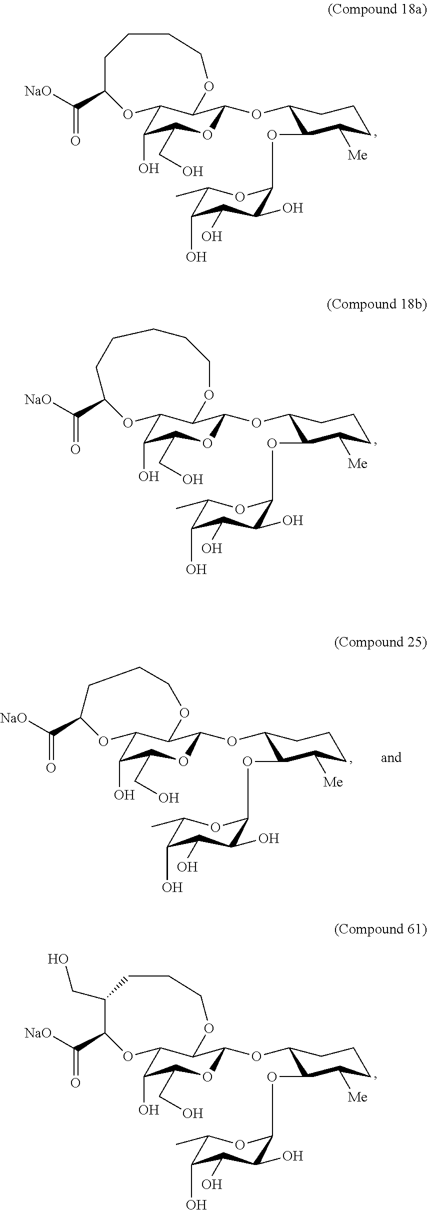

Also provided are pharmaceutical compositions comprising at least one compound of Formula (I). Such pharmaceutical compositions are described in greater detail herein. These compounds and compositions may be used in the methods described herein.

In some embodiments, at least one compound of Formula (I) or a pharmaceutical composition comprising at least one compound of Formula (I) may be used in methods described herein for decreasing the likelihood of occurrence of metastasis of cancer cells (also called tumor cells herein) in a subject (i.e., individual, patient) who is in need thereof by administering the at least one compound or composition to the subject.

In some embodiments, at least one compound of Formula (I) or a pharmaceutical composition comprising at least one compound of Formula (I) may be used in methods for decreasing the likelihood of occurrence of infiltration of cancer cells into bone marrow in a subject who is in need thereof by administering the at least one compound or composition to the subject.

In some embodiments, methods are provided herein for inhibiting adhesion of a cancer cell that expresses a ligand of E-selectin to an endothelial cell expressing E-selectin on the cell surface of the endothelial cell wherein the method comprises contacting the endothelial cell and at least one compound of Formula (I) or a pharmaceutical composition comprising at least one compound of Formula (I) (i.e., in some manner permitting the compound or composition comprising the compound to interact with the endothelial cell) such that the compound interacts with E-selectin on the endothelial cell, thereby inhibiting binding of the cancer cell to the endothelial cell. In some embodiments, the endothelial cell is present in the bone marrow.

In some embodiments, a method is provided for treating a cancer in a subject in need thereof by administering at least one compound of Formula (I) or a pharmaceutical composition comprising at least one compound of Formula (I) to the subject. The compound (or composition comprising the compound) may be administered in conjunction with (i.e., as an adjunct therapy, which is also called adjunctive therapy) chemotherapy and/or radiation.

The chemotherapy and/or radiation therapy may be referred to as the primary anti-tumor or anti-cancer therapy that is being administered to the subject to treat the particular cancer. In some embodiments, a method is provided herein for reducing (i.e., inhibiting, diminishing) chemosensitivity and/or radiosensitivity of hematopoietic stem cells (HSC) to the chemotherapeutic drug(s) or radioactive therapy, respectively, in a subject in need thereof, comprising administering to the subject at least one compound of Formula (I) or a pharmaceutical composition comprising at least one compound of Formula (I).

In some embodiments, methods are provided for enhancing (i.e., promoting) survival of hematopoietic stem cells in a subject in need thereof, comprising administering at least one compound of Formula (I) or a pharmaceutical composition comprising at least one compound of Formula (I) to the subject.

In some embodiments, a compound of Formula (I) or a pharmaceutical composition comprising at least one compound of Formula (I) may be useful in methods for treating and/or preventing thrombosis. In certain embodiments, the method comprising inhibiting formation of a thrombus by administering at least one of the compounds described herein or a pharmaceutical composition comprising at least one such compound.

In some embodiments, the compounds described herein and pharmaceutical compositions comprising at least one such compound may be used for treating and/or preventing an inflammatory disease or disorder.

In some embodiments, at least one compound of Formula (I) or a pharmaceutical composition comprising at least one compound of Formula (I) may be used in methods described herein for decreasing the likelihood of occurrence of mucositis in a subject who is in need thereof by administering the at least one compound or composition to the subject.

In some embodiments, the compounds described herein and pharmaceutical compositions comprising at least one such compound may be used for treating and/or preventing mucositis.

In some embodiments, a compound of Formula (I) or a pharmaceutical composition comprising at least one compound of Formula (I) may be useful in methods for mobilizing cells from the bone marrow in a subject in need thereof.

In some embodiments, a compound of Formula (I) or a pharmaceutical composition comprising at least one compound of Formula (I) may be useful in methods for mobilizing hematopoietic cells from the bone marrow in a subject in need thereof. In some embodiments, the hematopoietic cells are chosen from hematopoietic stem cells and hematopoietic progenitor cells. In some embodiments, the hematopoietic cells are mature white blood cells.

In some embodiments, a compound of Formula (I) or a pharmaceutical composition comprising at least one compound of Formula (I) may be useful in methods for mobilizing tumor cells. In some embodiments, the tumor cells are hematologic tumor cells. In some embodiments, the tumor cells are malignant cells.

In some embodiments, a compound of Formula (I) or a pharmaceutical composition comprising at least one compound of Formula (I) may be used for treating at least one of the diseases, disorders, and conditions described herein or for the preparation or manufacture of a medicament for use in treating at least one of the diseases, disorders, and/or conditions described herein. Each of these methods and uses is described in greater detail.

### DEFINITIONS

Whenever a term in the specification is identified as a range (e.g., C₁₋₄ alkyl), the range independently discloses and includes each element of the range. As a non-limiting example, C₁₋₄ alkyls includes, independently, C₁ alkyls, C₂ alkyls, C₃ alkyls, and C₄ alkyls.

The term “at least one” refers to one or more, such as one, two, etc. For example, the term “at least one C₁₋₄ alkyl” refers to one or more C₁₋₄ alkyl groups, such as one C₁₋₄ alkyl group, two C₁₋₄ alkyl groups, etc.

The term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted.

The term “alkenyl” includes straight, branched, and cyclic hydrocarbon groups comprising at least one double bond. The double bond of an alkenyl group can be unconjugated or conjugated with another unsaturated group. Non-limiting examples of alkenyl groups include vinyl, allyl, butenyl, pentenyl, hexenyl, butadienyl, pentadienyl, hexadienyl, 2-ethylhexenyl, and cyclopent-1-en-1-yl. Unless stated otherwise specifically in the specification, an alkenyl group may be optionally substituted.

The term “alkynyl” includes straight and branched hydrocarbon groups comprising at least one triple bonds. The triple bond of an alkynyl group can be unconjugated or conjugated with another unsaturated group. Non-limiting examples of alkynyl groups include ethynyl, propynyl, butynyl, pentynyl, and hexynyl. Unless stated otherwise specifically in the specification, an alkynyl group may be optionally substituted.

The term “alkylene” includes straight, branched, and cyclic divalent hydrocarbon groups. The alkylene group can be saturated or unsaturated (i.e., contains one or more double and/or triple bonds) comprising from 2 to 12 carbon atoms. The points of attachment of the alkylene group to the rest of the molecule can be through one carbon or any two carbons within the group. Unless stated otherwise specifically in the specification, an alkylene group may be optionally substituted

The term “alkoxy” includes —ORₐ groups wherein Rₐ is chosen from alkyl, alkenyl and akynyl groups as defined herein. Non-limiting examples of alkoxy include methoxy, ethoxy, propoxy, isopropoxy, tert-butoxy, and neopentyloxy. Unless stated otherwise specifically in the specification, an alkoxy group may be optionally substituted.

The term “alkylamino” includes —NHRₐ and —NRₐRₐ groups wherein each Rₐ, which may be identical or different, is independently chosen from alkyl, alkenyl, and alkynyl groups as defined herein or, taken together, may form a ring chosen from 4- to 12-membered monocyclic rings, 4- to 12-membered bicyclic rings, 4- to 12-membered tricyclic rings, and 4- to 12-membered benzofused rings. Unless stated otherwise specifically in the specification, an alkylamino group may be optionally substituted.

The term “aryl” includes hydrocarbon ring system group comprising 6 to 18 carbon ring atoms and at least one aromatic ring. The aryl group may be a monocyclic, bicyclic, tricyclic or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The term “arylalkyl” or “aralkyl” includes aryl groups, as described herein, appended to the parent molecular moiety through an alkyl group, as defined herein. Non-limiting examples of an arylalkyl or aralkyl group include benzyl, phenethyl, and diphenylmethyl. Unless stated otherwise specifically in the specification, an arylalkyl or aralkyl group may be optionally substituted.

The term “cycloalkyl” or “carbocyclic ring” includes saturated monocyclic or polycyclic hydrocarbon group, which may include fused or bridged ring systems. Non-limiting examples of a cycloalkyl group include cyclopropyl, cyclobutyl, cyclopentyl, cyclohexyl, cycloheptyl, cyclooctyl, adamantyl, and norbornyl. Unless otherwise stated specifically in the specification, a cycloalkyl group may be optionally substituted.

The term “E-selectin antagonist” includes inhibitors of E-selectin only, as well as inhibitors of E-selectin and either P-selectin or L-selectin, and inhibitors of E-selectin, P-selectin, and L-selectin.

The term “fused” includes any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

The term “glycomimetic” includes any naturally occurring or non-naturally occurring carbohydrate compound in which at least one substituent has been replaced, or at least one ring has been modified (e.g., substitution of carbon for a ring oxygen), to yield a compound that is not fully carbohydrate.

The term “halo” or “halogen” includes fluoro, chloro, bromo, and iodo.

The term “haloalkyl” includes alkyl groups, as defined herein, substituted by at least one halogen, as defined herein. Non-limiting examples include trifluoromethyl, difluoromethyl, trichloromethyl, 2,2,2-trifluoroethyl, 1,2-difluoroethyl, 3-bromo-2-fluoropropyl, and 1,2-dibromoethyl. A “fluoroalkyl” is a haloalkyl that is substituted with at least one fluoro group. Unless stated otherwise specifically in the specification, a haloalkyl group may be optionally substituted.

The term “haloalkenyl” includes alkenyl groups, as defined herein, substituted by at least one halogen, as defined herein. Non-limiting examples include fluoroethenyl, 1,2-difluoroethenyl, 3-bromo-2-fluoropropenyl, and 1,2-dibromoethenyl. A “fluoroalkenyl” is a haloalkenyl substituted with at least one fluoro group. Unless stated otherwise specifically in the specification, a haloalkenyl group may be optionally substituted.

The term “haloalkynyl” includes alkynyl groups, as defined herein, substituted by at least one halogen, as defined herein. Non-limiting examples include fluoroethynyl, 1,2-difluoroethynyl, 3-bromo-2-fluoropropynyl, and 1,2-dibromoethynyl. A “fluoroalkynyl” is a haloalkynyl substituted with at least one fluoro group. Unless stated otherwise specifically in the specification, a haloalkynyl group may be optionally substituted.

The term “halocycloalkyl” includes cycloalkyl groups as defined herein, substituted by at least one halogen. Non-limiting examples include fluorocyclopropane and fluorocyclohexane. Unless stated otherwise specifically in the specification, a halocycloalkyl group may be optionally substituted.

The term “heterocyclyl” or “heterocyclic ring” includes 3- to 18-membered saturated or partially unsaturated non-aromatic ring groups comprising 2 to 12 ring carbon atoms and 1 to 6 ring heteroatom(s) each independently chosen from N, O, and S. Unless stated otherwise specifically in the specification, the heterocyclyl groups may be a monocyclic, bicycle, tricyclic or tetracyclic ring system, which may include fused or bridged ring systems; and the nitrogen, carbon or sulfur atoms in the heterocyclyl group may be optionally oxidized; the nitrogen atom may be optionally quaternized; and the heterocyclyl group may be partially or fully saturated. Non-limiting examples include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “heterocyclylalkyl” includes —R_{b}Rₑ groups wherein R_{b} is an alkyl group as defined herein and Rₑ is a heterocyclyl group as defined herein, and if the heterocyclyl is a nitrogen-containing heterocyclyl, the heterocyclyl may be attached to the alkyl group at the nitrogen atom. Unless stated otherwise specifically in the specification, a heterocyclylalkyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

The term “hydroxylalkyl,” “hydroxylalkenyl” and “hydroxylalkynyl” includes alkyl, alkenyl or alkynyl groups, respectively, as defined herein, substituted by at least one hydroxyl group. The hydroxyl groups may be primary, secondary or tertiary. Unless stated otherwise specifically in the specification, a hydroxylalkyl, hydroxylalkenyl and hydroxylalkynyl group may be optionally substituted.

The term “non-glycomimetic moiety” includes moieties having a structure not intended to mimic a carbohydrate molecule. A non-glycomimetic moiety may not be (and is typically not) active as an E selectin antagonist. Instead, non-glycomimetic moieties are generally moieties added to a glycomimetic moiety for purposes of altering at least one property such as solubility, bio-availability, lipophilicity and/or other drug-like properties of the glycomimetic. Non-limiting examples of a non-glycomimetic moiety include steroidal compounds such as, for example, cholic acid, fatty acids, lipids, and amphiphilic compounds. For example, a non-glycomimetic moiety may be chosen from C₂₋₂₅ alkyls, C₂₋₂₅ alkenyls, C₂₋₂₅ alkynyls, C₁₋₂₅ haloalkyls, C₂₋₂₅ haloalkenyls, C₂₋₂₅ haloalkynyls, polycyclic cycloalkyls, fatty acids, lipids, steroids, and non-sulfonated amphiphiles.

The term “non-sulfonated amphiphile” is an amphiphile that does not include sulfonate groups.

The term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

The term “steroid” or “steroidal moiety” includes compounds and moieties that contain a characteristic arrangement of four cycloalkane rings that are joined to each other. The core of a steroid comprises twenty carbon atoms bonded together that take the form of four fused rings: three cyclohexane rings and one cyclopentane ring. Non-limiting examples of a steroidal moiety include cholic acid, cholesterol and derivatives thereof.

The term “spirocyclic” includes compounds comprising at least two cyclic moieties (e.g., cycloalkyl groups) joined through a single atom (e.g., carbon atom).

The term “substituted” includes the situation where, in any of the above groups, at least one hydrogen atom is replaced by a non-hydrogen atom such as, for example, a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

The term “thioalkyl” includes —SRₐ groups wherein Rₐ is chosen from alkyl, alkenyl, and alkynyl groups, as defined herein. Unless stated otherwise specifically in the specification, a thioalkyl group may be optionally substituted.

The present disclosure includes within its scope all the possible geometric isomers, e.g. Z and E isomers (cis and trans isomers), of the compounds as well as all the possible optical isomers, e.g. diastereomers and enantiomers, of the compounds. Furthermore, the present disclosure includes in its scope both the individual isomers and any mixtures thereof, e.g. racemic mixtures. The individual isomers may be obtained using the corresponding isomeric forms of the starting material or they may be separated after the preparation of the end compound according to conventional separation methods. For the separation of optical isomers, e.g. enantiomers, from the mixture thereof conventional resolution methods, e.g. fractional crystallization, may be used.

The present disclosure includes within its scope all possible tautomers. Furthermore, the present disclosure includes in its scope both the individual tautomers and any mixtures thereof.

### Compound Synthesis Procedures

Compounds of Formula (I) may be prepared according to General Reaction Scheme I below. It is understood that one of ordinary skill in the art may be able to make these compounds by similar methods or by combining other methods known to one of ordinary skill in the art. It is also understood that one of ordinary skill in the art would be able to make, in a similar manner as described below, other compounds of Formula (I) not specifically illustrated herein by using appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. and/or synthesized according to sources known to those of ordinary skill in the art (see, for example, Advanced Organic Chemistry: Reactions, Mechanisms, and Structure, 5th edition (Wiley, December 2000)) and/or prepared as described herein.

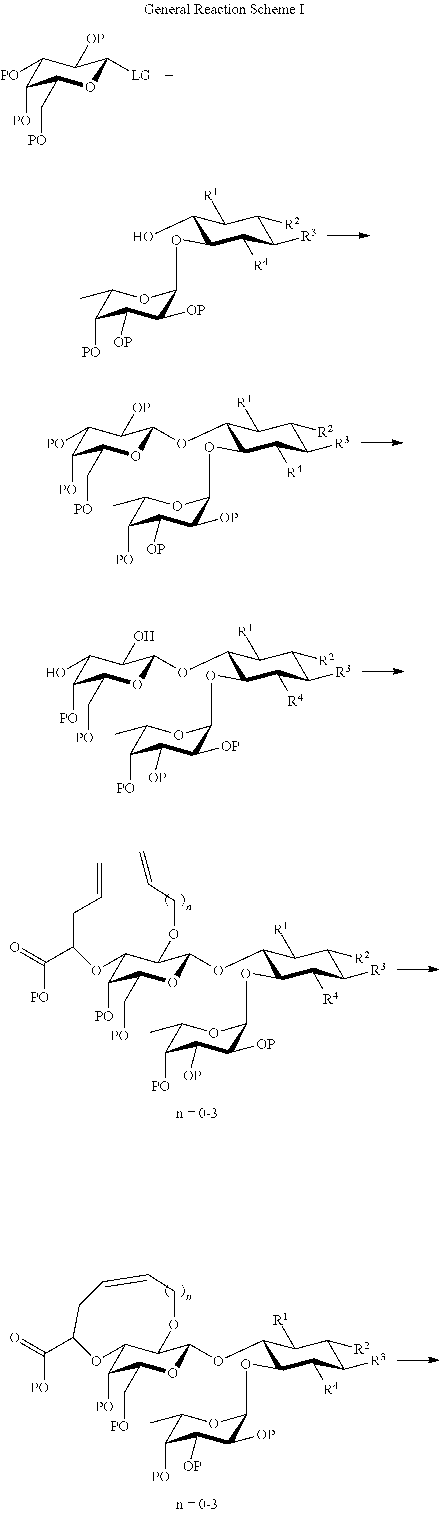

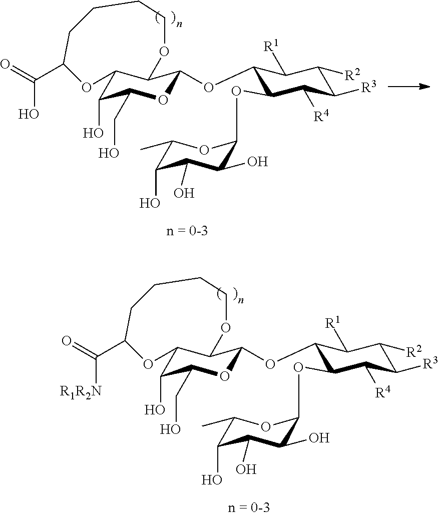

Those of ordinary skill in the art will understand that, in processes described herein, the functional groups of intermediate compounds may need to be protected by at least one suitable protecting group. Non-limiting examples of such functional groups include, hydroxyl groups, amino groups, mercapto groups, and carboxylic acid groups. Non-limiting examples of suitable protecting groups for hydroxy groups include trialkylsilyl and diarylalkylsilyl groups (for example, t-butyldimethylsilyl, t-butyldiphenylsilyl or trimethylsilyl), tetrahydropyranyl, and benzyl. Non-limiting examples of suitable protecting groups for amino, amidino and guanidino include t-butoxycarbonyl and benzyloxycarbonyl groups. Non-limiting examples of suitable protecting groups for mercapto include —C(O)—R″ (where R″ is alkyl, aryl or arylalkyl), p-methoxybenzyl, and trityl groups. Non-limiting examples of suitable protecting groups for carboxylic acid include alkyl, aryl and arylalkyl esters. Protecting groups may be added or removed in accordance with standard techniques, which are known to one of ordinary skill in the art and as described herein. The use of protecting groups is, for example, described in detail in Green, T. W. and P. G. M. Wutz, Protective Groups in Organic Synthesis (1999), 3rd Ed., Wiley. As one of ordinary skill in the art would appreciate, the protecting group may also be a polymer resin such as a Wang resin, Rink resin or a 2-chlorotrityl-chloride resin.

### Methods for Characterizing Glycomimetic Compounds

Biological activity of a glycomimetic compound described herein may be determined, for example, by performing at least one in vitro and/or in vivo study routinely practiced in the art and described herein or in the art. In vitro assays include without limitation binding assays, immunoassays, competitive binding assays and cell based activity assays.

An inhibition assay may be used to screen for antagonists of E-selectin. For example, an assay may be performed to characterize the capability of a compound described herein to inhibit (i.e., reduce, block, decrease, or prevent in a statistically or biologically significant manner) interaction of E-selectin with sLe^{a} or sLe^{x}. The inhibition assay may be a competitive binding assay, which allows the determination of IC₅₀ values. By way of example, E-selectin/Ig chimera may be immobilized onto a matrix (e.g., a multi-well plate, which may be made from a polymer, such as polystyrene; a test tube, and the like); a composition may be added to reduce nonspecific binding (e.g., a composition comprising non-fat dried milk or bovine serum albumin or other blocking buffer routinely used by a person skilled in the art); the immobilized E-selectin may be contacted with the candidate compound in the presence of sLe^{a} comprising a reporter group under conditions and for a time sufficient to permit sLe^{a} to bind to the immobilized E-selectin; the immobilized E-selectin may be washed; and the amount of sLe^{a} bound to immobilized E-selectin may be detected. Variations of such steps can be readily and routinely accomplished by a person of ordinary skill in the art.

Conditions for a particular assay include temperature, buffers (including salts, cations, media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

The source of a compound that is characterized by at least one assay and techniques described herein and in the art may be a biological sample that is obtained from a subject who has been treated with the compound. The cells that may be used in the assay may also be provided in a biological sample. A “biological sample” may include a sample from a subject, and may be a blood sample (from which serum or plasma may be prepared), a biopsy specimen, one or more body fluids (e.g., lung lavage, ascites, mucosal washings, synovial fluid, urine), bone marrow, lymph nodes, tissue explant, organ culture, or any other tissue or cell preparation from the subject or a biological source. A biological sample may further include a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. In some embodiments, the subject or biological source may be a human or non-human animal, a primary cell culture (e.g., immune cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

As described herein, methods for characterizing E-selectin antagonists include animal model studies. Non-limiting examples of animal models for liquid cancers used in the art include multiple myeloma (see, e.g., DeWeerdt, Nature 480:S38S39 (15 Dec. 2011) doi:10.1038/480S38a; Published online 14 Dec. 2011; Mitsiades et al., Clin. Cancer Res. 2009 15:1210021 (2009)); acute myeloid leukemia (AML) (Zuber et al., Genes Dev. 2009 Apr. 1; 23(7): 877-889). Animal models for acute lymphoblastic leukemia (ALL) have been used by persons of ordinary skill in the art for more than two decades. Numerous exemplary animal models for solid tumor cancers are routinely used and are well known to persons of ordinary skill in the art.

Methods for Treating and/or Preventing Diseases, Disorders, or Conditions

The compounds of the present disclosure and the pharmaceutical compositions comprising at least one of such compounds may be useful in methods for treating and/or preventing a disease or disorder that is treatable by inhibiting at least one activity of E-selectin (and/or inhibiting binding of E-selectin to a ligand, which in turn inhibits a biological activity). Focal adhesion of leukocytes to the endothelial lining of blood vessels is a characteristic step in certain vascular disease processes.

The compounds of the present disclosure and pharmaceutical compositions comprising at least one such compound may be useful in methods for treating and/or preventing at least one inflammatory disease. Inflammation comprises reaction of vascularized living tissue to injury. By way of example, although E-selectin-mediated cell adhesion is important to the body's anti-infective immune response, in other circumstances, E-selectin mediated cell adhesion may be undesirable or excessive, resulting in tissue damage instead of repair. For example, many pathologies (such as autoimmune and inflammatory diseases, shock and reperfusion injuries) involve abnormal adhesion of white blood cells. Therefore, inflammation affects blood vessels and adjacent tissues in response to an injury or abnormal stimulation by a physical, chemical, or biological agent. Examples of inflammatory diseases, disorders, or conditions include, without limitation, dermatitis, chronic eczema, psoriasis, multiple sclerosis, rheumatoid arthritis, systemic lupus erythematosus, graft versus host disease, sepsis, diabetes, atherosclerosis, Sjogren's syndrome, progressive systemic sclerosis, scleroderma, acute coronary syndrome, ischemic reperfusion, Crohn's disease, inflammatory bowel disease, endometriosis, glomerulonephritis, myasthenia gravis, idiopathic pulmonary fibrosis, asthma, allergic reaction, acute respiratory distress syndrome (ARDS) or other acute leukocyte-mediated lung injury, vasculitis, or inflammatory autoimmune myositis. Other diseases and disorders for which the glycomimetic compounds described herein may be useful for treating and/or preventing include hyperactive coronary circulation, microbial infection, cancer metastasis, thrombosis, wounds, burns, spinal cord damage, digestive tract mucous membrane disorders (e.g., gastritis, ulcers), osteoporosis, osteoarthritis, septic shock, traumatic shock, stroke, nephritis, atopic dermatitis, frostbite injury, adult dyspnoea syndrome, ulcerative colitis, diabetes and reperfusion injury following ischaemic episodes, prevention of restinosis associated with vascular stenting, and for undesirable angiogenesis, for example, angiogenesis associated with tumor growth.

As understood by a person of ordinary skill in the medical art, the terms, “treat” and “treatment,” include medical management of a disease, disorder, or condition of a subject (i.e., patient, individual) (see, e.g., Stedman's Medical Dictionary). In general, an appropriate dose and treatment regimen provide at least one of the compounds of the present disclosure in an amount sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or retard (lessen) an undesired physiological change or disorder, or to prevent or slow or retard (lessen) the expansion or severity of such disorder. As discussed herein, beneficial or desired clinical results from treating a subject include, but are not limited to, abatement, lessening, or alleviation of symptoms that result from or are associated with the disease, condition, or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can include prolonging survival when compared to expected survival if a subject were not receiving treatment. Subjects in need of treatment include those who already have the disease, condition, or disorder as well as subjects prone to have or at risk of developing the disease, condition, or disorder, and those in which the disease, condition, or disorder is to be prevented (i.e., decreasing the likelihood of occurrence of the disease, disorder, or condition).

In some embodiments of the methods described herein, the subject is a human. In some embodiments of the methods described herein, the subject is a non-human animal. A subject in need of treatment as described herein may exhibit at least one symptom or sequelae of the disease, disorder, or condition described herein or may be at risk of developing the disease, disorder, or condition. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The effectiveness of the compounds of the present disclosure in treating and/or preventing a disease, disorder, or condition described herein can readily be determined by a person of ordinary skill in the medical and clinical arts. Determining and adjusting an appropriate dosing regimen (e.g., adjusting the amount of compound per dose and/or number of doses and frequency of dosing) can also readily be performed by a person of ordinary skill in the medical and clinical arts. One or any combination of diagnostic methods, including physical examination, assessment and monitoring of clinical symptoms, and performance of analytical tests and methods described herein, may be used for monitoring the health status of the subject.

### Methods for Treating or Preventing Binding of Cancer Cells to E-Selectin and for Treating Cancer and Metastasis

As discussed in detail herein, a disease or disorder to be treated or prevented is a cancer and related metastasis and includes cancers that comprise solid tumor(s) and cancers that comprise liquid tumor(s). The compounds of the present disclosure and pharmaceutical compositions comprising at least one such compound may be useful in methods for preventing and/or treating cancer. In some embodiments, the at least one compound may be used for treating and/or preventing metastasis and/or for inhibiting (slowing, retarding, or preventing) metastasis of cancer cells.

In some embodiments, the compounds of present disclosure and pharmaceutical compositions comprising at least one such compound may be used for decreasing (i.e., reducing) the likelihood of occurrence of metastasis of cancer cells in an individual (i.e., subject, patient) who is in need thereof. The compounds of the present disclosure and compositions comprising at least one such compound may be used for decreasing (i.e., reducing) the likelihood of occurrence of infiltration of cancer cells into bone marrow in an individual who is in need thereof. The individuals (or subjects) in need of such treatments include subjects who have been diagnosed with a cancer, which includes cancers that comprise solid tumor(s) and cancers that comprise liquid tumor(s).

Non-limiting examples of cancers include colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, melanoma, breast cancer, and pancreatic cancer. Liquid tumors can occur in the blood, bone marrow, the soft, sponge-like tissue in the center of most bones, and lymph nodes and include leukemia (e.g., AML, ALL, CLL, and CML), lymphoma, and myeloma (e.g., multiple myeloma). Lymphomas include Hodgkin lymphoma, which is marked by the presence of a type of cell called the Reed-Sternberg cell, and non-Hodgkin lymphomas, which includes a large, diverse group of cancers of immune system cells. Non-Hodgkin lymphomas can be further divided into cancers that have an indolent (slow-growing) course and those that have an aggressive (fast-growing) course, and which subtypes respond to treatment differently.

The compounds of the present disclosure and pharmaceutical compositions comprising at least one such compound may be administered as an adjunct therapy to chemotherapy and/or radiotherapy, which is/are being delivered to the subject as primary therapy for treating the cancer. The chemotherapy and/or radiotherapy that may be administered depend upon several factors including the type of cancer, location of the tumor(s), stage of the cancer, age and gender and general health status of the subject. A person of ordinary skill in the medical art can readily determine the appropriate chemotherapy regimen and/or radiotherapy regimen for the subject in need. The person of ordinary skill in the medical art can also determine, with the aid of preclinical and clinical studies, when the compound of the present disclosure or pharmaceutical composition comprising at least one such compound should be administered to the subject, that is whether the compound or composition is administered prior to, concurrent with, or subsequent to a cycle of the primary chemotherapy or radiation treatment.

Also provided herein is a method for inhibiting adhesion of a tumor cell that expresses a ligand of E-selectin to an endothelial cell expressing E-selectin on its cell surface, which method comprises contacting the endothelial cell with at least one compound of the present disclosure or pharmaceutical compositions comprising at least one such compound, thereby permitting the compound to interact with E-selectin on the endothelial cell surface and inhibiting binding of the tumor cell to the endothelial cell. Without wishing to be bound by theory, inhibiting adhesion of tumor cells to endothelial cells may reduce in a significant manner, the capability of the tumor cells to extravasate into other organs, blood vessels, lymph, or bone marrow and thereby reduce, decrease, or inhibit, or slow the progression of the cancer, including reducing, decreasing, inhibiting, or slowing metastasis.

As described herein, at least one of the compounds of the present disclosure or pharmaceutical compositions comprising at least one such compound may be administered in combination with at least one additional anti-cancer agent. Chemotherapy may comprise one or more chemotherapeutic agents. For example, chemotherapy agents, radiotherapeutic agents, inhibitors of phosphoinositide-3 kinase (PI3K), and inhibitors of VEGF may be used in combination with an E-selectin antagonist compound described herein. Non-limiting examples of inhibitors of PI3K include the compound named by Exelixis as “XL499.” Non-limiting examples of VEGF inhibitors include the compound called “cabo” (previously known as XL184). Many other chemotherapeutics are small organic molecules. As understood by a person of ordinary skill in the art, chemotherapy may also refer to a combination of two or more chemotherapeutic molecules that are administered coordinately and which may be referred to as combination chemotherapy. Numerous chemotherapeutic drugs are used in the oncology art and include, for example, alkylating agents; antimetabolites; anthracyclines, plant alkaloids; and topoisomerase inhibitors.

The compounds of the present disclosure or pharmaceutical compositions comprising at least one such compound may function independently from the anti-cancer agent or may function in coordination with the anti-cancer agent, e.g., by enhancing effectiveness of the anti-cancer agent or vice versa. Accordingly, provided herein are methods for enhancing (i.e., enhancing, promoting, improving the likelihood of, enhancing in a statistically or biologically significant manner) and/or maintaining survival of hematopoietic stem cells (HSC) in a subject who is treated with and/or will be treated with a chemotherapeutic drug(s) and/or radioactive therapy, respectively, comprising administering at least one E-selectin antagonist glycomimetic compound as described herein. In some embodiments, the subject receives and/or will receive both chemotherapy and radiation therapy. Also, provided herein is a method for reducing (i.e., reducing, inhibiting, diminishing in a statistically or biologically significant manner) chemosensitivity and/or radiosensitivity of hematopoietic stem cells (HSC) to the chemotherapeutic drug(s) and/or radioactive therapy, respectively, in a subject. Because repeated cycles of chemotherapy and radiotherapy often diminish the ability of HSCs to recover and replenish bone marrow, the glycomimetic compounds described herein may be useful for subjects who will receive more than one cycle, such as at least two, three, four or more cycles, of chemotherapy and/or radiotherapy. HSCs reside in the bone marrow and generate the cells that are needed to replenish the immune system and the blood. Anatomically, bone marrow comprises a vascular niche that is adjacent to bone endothelial sinuses (see, e.g., Kiel et al., Cell 121:1109-21 (2005); Sugiyama et al., Immunity 25:977-88 (2006); Mendez-Ferrer et al., Nature 466:829-34 (2010); Butler et al., Cell Stem Cell 6:251-64 (2010)). A recent study describes that E-selectin promotes HSC proliferation and is an important component of the vascular niche (see, e.g., Winkler et al., Nature Medicine published online 21 Oct. 2012; doi:10.1038/nm.2969). Deletion or inhibition of E-selectin enhanced HSC survival in mice that were treated with chemotherapeutic agents or radiotherapy and accelerated blood neutrophil recovery (see, e.g., Winkler et al., supra).

In addition, the administration of at least one compound of the present disclosure or pharmaceutical composition comprising at least one such compounds may be in conjunction with one or more other therapies, e.g., for reducing toxicities of therapy. For example, at least one palliative agent to counteract (at least in part) a side effect of a therapy (e.g., anti-cancer therapy) may be administered. Agents (chemical or biological) that promote recovery, or counteract side effects of administration of antibiotics or corticosteroids, are examples of such palliative agents. At least one E-selectin antagonist described herein may be administered before, after, or concurrently with administration of at least one additional anti-cancer agent or at least one palliative agent to reduce a side effect of therapy. When administration is concurrent, the combination may be administered from a single container or two (or more) separate containers.

Cancer cells (also called herein tumor cells) that may be prevented (i.e., inhibited, slowed) from metastasizing, from adhering to an endothelial cell, or from infiltrating bone marrow include cells of solid tumors and liquid tumors (including hematological malignancies). Examples of solid tumors are described herein and include colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, melanoma, breast cancer, and pancreatic cancer. Liquid tumors occur in the blood, bone marrow, and lymph nodes and include leukemia (e.g., AML, ALL, CLL, and CML), lymphoma (e.g., Hodgkin lymphoma and non-Hodgkin lymphoma), and myeloma (e.g., multiple myeloma). As used herein, the term cancer cells include mature, progenitor, and cancer stem cells.

Bones are a common location for cancer to infiltrate once leaving the primary tumor location. Once cancer resides in bone, it is frequently a cause of pain to the individual. In addition, if the particular bone affected is a source for production of blood cells in the bone marrow, the individual may develop a variety of blood cell related disorders. Breast and prostate cancer are examples of solid tumors that migrate to bones. Acute myelogenous leukemia (AML) and multiple myeloma (MM) are examples of liquid tumors that migrate to bones. Cancer cells that migrate to bone will typically migrate to the endosteal region of the bone marrow. Once cancer cells have infiltrated into the marrow, the cells become quiescent and are protected from chemotherapy. The compounds of the present disclosure block infiltration of disseminated cancer cells into bone marrow. A variety of individuals may benefit from treatment with the compounds. Examples of such individuals include individuals with a cancer type having a propensity to migrate to bone where the tumor is still localized or the tumor is disseminated but not yet infiltrated bone, or where individuals with such a cancer type are in remission.

The cancer patient population most likely to respond to treatment using the E-selectin antagonist agents (e.g., compounds of Formula (I)) described herein can be identified based on the mechanism of action of E-selectin. That is, patients may be selected that express a highly active E-selectin as determined by the genetic polymorphism for E-selectin of S128R (Alessandro et al., Int. J. Cancer 121:528-535, 2007). In addition, patients for treatment by the compounds described herein may also selected based on elevated expression of the E-selectin binding ligands (sialyl Le^{a} and sialyl Le^{x}) as determined by antibodies directed against cancer-associated antigens CA-19-9 (Zheng et al., World J. Gastroenterol. 7:431-434, 2001) and CD65. In addition, antibodies HECA-452 and FH-6 which recognize similar carbohydrate ligands of E-selectin may also be used in a diagnostic assay to select the cancer patient population most likely to respond to this treatment.

### Methods for Treating or Preventing Thrombus Formation

The compounds of the present disclosure and pharmaceutical compositions comprising at least one such compound may be useful in methods for treating and/or preventing thrombosis. As described herein methods are provided for inhibiting formation of a thrombus or inhibiting the rate at which a thrombus is formed. These methods may therefore be used for preventing thrombosis (i.e., reducing or decreasing the likelihood of occurrence of a thrombus in a statistically or clinically significant manner).

Thrombus formation may occur in infants, children, teenagers and adults. An individual may have a hereditary predisposition to thrombosis. Thrombosis may be initiated, for example, due to a medical condition (such as cancer or pregnancy), a medical procedure (such as surgery) or an environmental condition (such as prolonged immobility). Other individuals at risk for thrombus formation include those who have previously presented with a thrombus.

The compounds of the present disclosure and pharmaceutical compositions comprising at least one such compound may be useful in methods for treating individuals undergoing thrombosis or who are at risk of a thrombotic event occurring. Such individuals may or may not have a risk of bleeding. In some embodiments, the individual has a risk of bleeding. In some embodiments, the thrombosis is a venous thromboembolism (VTE). VTE causes deep vein thrombosis and pulmonary embolism. Low molecular weight (LMW) heparin is the current mainstay therapy for the prevention and treatment of VTE. In many circumstances, however, the use of LMW heparin is contraindicated. LMW heparin is a known anti-coagulant and delays clotting over four times longer than control bleeding times. Patients undergoing surgery, patients with thrombocytopenia, patients with a history of stroke, and many cancer patients should avoid administration of heparin due to the risk of bleeding. By contract, administration of the E-selectin antagonist compounds of Formula (I) significantly reduces the time to clotting than occurs when LMW heparin is administered, and thus provide a significant improvement in reducing bleeding time compared with LMW heparin. Accordingly, the compounds and pharmaceutical compositions described herein may not only be useful for treating a patient for whom the risk of bleeding is not significant, but also may be useful in when the risk of bleeding is significant and the use of anti-thrombosis agents with anti-coagulant properties (such as LMW heparin) is contraindicated.

The compounds of the present disclosure and pharmaceutical compositions comprising at least one such compound may be administered in combination with at least one additional anti-thrombosis agent. The compounds of the present disclosure and pharmaceutical compositions comprising at least one such compound may function independently from the anti-thrombosis agent or may function in coordination with the at least one anti-thrombosis agent. In addition, the administration of one or more of the compounds or compositions may be in conjunction with one or more other therapies, e.g., for reducing toxicities of therapy. For example, at least one palliative agent to counteract (at least in part) a side effect of therapy may be administered. Agents (chemical or biological) that promote recovery and/or counteract side effects of administration of antibiotics or corticosteroids are examples of such palliative agents. The compounds of the present disclosure and pharmaceutical composition comprising at least one such compound may be administered before, after, or concurrently with administration of at least one additional anti-thrombosis agent or at least one palliative agent to reduce a side effect of therapy. Where administration is concurrent, the combination may be administered from a single container or two (or more) separate containers.

Methods for Treating and/or Preventing Mucositis

The compounds of the present disclosure and pharmaceutical compositions comprising at least one such compound may be useful in methods for preventing and/or treating mucositis. In some embodiments, at least one compound of Formula (I) or a pharmaceutical composition comprising at least one compound of Formula (I) may be used in methods described herein for decreasing the likelihood of occurrence of mucositis in a subject who is in need thereof by administering the compound or composition to the subject. In some embodiments, the mucositis is chosen from oral mucositis, esophageal mucositis, and gastrointestinal mucositis. In some embodiments, the mucositis is alimentary mucositis.

It is believed that approximately half of all cancer patients undergoing therapy suffer some degree of mucositis. Mucositis is believed to occur, for example, in virtually all patients treated with radiation therapy for head and neck tumors, all patients receiving radiation along the GI tract, and approximately 40% of those subjected to radiation therapy and/or chemotherapy for tumors in other locations (e.g., leukemias or lymphomas). It is also is believed to be highly prevalent in patients treated with high dose chemotherapy and/or irradiation for the purpose of myeloablation, such as in preparation for stem cell or bone marrow transplantation. The compounds of the present disclosure and pharmaceutical compositions comprising at least one such compound may be useful in methods for treating and/or preventing mucositis in a subject afflicted with cancer. In some embodiments, the subject is afflicted with a cancer chosen from head and neck cancer, breast cancer, lung cancer, ovarian cancer, prostate cancer, lymphatic cancer, leukemic cancer, and/or gastrointestinal cancer. In some embodiments, the mucositis is associated with radiation therapy and/or chemotherapy. In some embodiments, the chemotherapy comprises administering a therapeutically effective amount of at least one compound chosen from platinum, cisplatin, carboplatin, oxaliplatin, mechlorethamine, cyclophosphamide, chlorambucil, azathioprine, mercaptopurine, vincristine, vinblastine, vinorelbine, vindesine, etoposide, teniposide, paclitaxel, docetaxel, irinotecan, topotecan, amsacrine, etoposide, etoposide phosphate, teniposide, 5-fluorouracil (5-FU), leucovorin, methotrexate, gemcitabine, taxane, leucovorin, mitomycin C, tegafur-uracil, idarubicin, fludarabine, mitoxantrone, ifosfamide and doxorubicin.

In some embodiments, the method further comprising a therapeutically effective amount of at least one MMP inhibitor, inflammatory cytokine inhibitor, mast cell inhibitor, NSAID, NO inhibitor, or antimicrobial compound.

In some embodiments, the method further comprising a therapeutically effective amount of velafermin and/or palifermin.

Methods for Mobilizing Cells from the Bone Marrow

The compounds of the present disclosure and pharmaceutical compositions comprising at least one such compound may be useful in methods for mobilizing cells from the bone marrow to the peripheral vasculature and tissues. As discussed herein, in some embodiments, the compounds and compositions are useful for mobilizing hematopoietic cells, including hematopoietic stem cells and hematopoietic progenitor cells. In some embodiments, the compounds act as mobilizing agents of normal blood cell types. In some embodiments, the agents are used in methods for mobilizing mature white blood cells (which may also be called leukocytes herein), such as granulocytes (e.g., neutrophils, eosinophils, basophils), lymphocytes, and monocytes from the bone marrow or other immune cell compartments such as the spleen and liver. Methods are also provided for using the compounds of the present disclosure and pharmaceutical compositions comprising at least one such compound in methods for mobilizing tumor cells from the bone marrow. The tumor cells may be malignant cells (e.g., tumor cells that are metastatic cancer cells, or highly invasive tumor cells) in cancers. These tumor cells may be of hematopoietic origin or may be malignant cells of another origin residing in the bone.

In some embodiments, the methods using the E-selectin antagonists described herein are useful for mobilizing hematopoietic cells, such as hematopoietic stem cells and progenitor cells and leukocytes (including granulocytes such as neutrophils), which are collected (i.e., harvested, obtained) from the subject receiving the E-selectin antagonist and at a later time are administered back into the same subject (autologous donor) or administered to a different subject (allogeneic donor). Hematopoietic stem cell replacement and hematopoietic stem cell transplantation have been successfully used for treating a number of diseases (including cancers) as described herein and in the art. By way of example, stem cell replacement therapy or transplantation follows myeloablation of a subject, such as occurs with administration of high dose chemotherapy and/or radiotherapy. Desirably, an allogeneic donor shares sufficient HLA antigens with the recipient/subject to minimize the risk of host versus graft disease in the recipient (i.e., the subject receiving the hematopoietic stem cell transplant). Obtaining the hematopoietic cells from the donor subject (autologous or allogeneic) is performed by apheresis or leukapheresis. HLA typing of a potential donor and the recipient and apheresis or leukapheresis are methods routinely practiced in the clinical art.

By way of non-limiting example, autologous or allogenic hematopoietic stem cells and progenitors cells may be used for treating a recipient subject who has certain cancers, such as Hodgkin lymphoma, non-Hodgkin lymphoma, or multiple myeloma. Allogeneic hematopoietic stem cells and progenitors cells may be used, for example, for treating a recipient subject who has acute leukemias (e.g., AML, ALL); chronic lymphocytic leukemia (CLL); amegakaryocytosis/congenital thrombocytopenia; aplastic anemia/refractory anemia; familial erythrophagocytic lymphohistiocytosis; myelodysplastic syndrome/other myelodysplastic disorders; osteopetrosis; paroxysmal nocturnal hemoglobinuria; and Wiskott-aldrich syndrome, for example. Exemplary uses for autologous hematopoietic stem cells and progenitors cells include treating a recipient subject who has amyloidosis; germ cell tumors (e.g., testicular cancer); or a solid tumor. Allogeneic hematopoietic stem cell transplants have also been investigated for use in treating solid tumors (see, e.g., Ueno et al., Blood 102:3829-36 (2003)).

In some embodiments of the methods described herein, the subject is not a donor of peripheral hematopoietic cells but has a disease, disorder, or condition for which mobilization of hematopoietic cells in the subject will provide clinical benefit. Stated another way, while this clinical situation is similar to autologous hematopoietic cell replacement, the mobilized hematopoeitic cells are not removed and given back to the same subject at a later time as occurs, for example, with a subject who receives myeloablation therapy. Accordingly, methods are provided for mobilizing hematopoietic cells, such as hematopoietic stem cells and progenitor cells and leukocytes (including granulocytes, such as neutrophils), by administering at least once compound of Formula (I). Mobilizing hematopoietic stem cells and progenitor cells may be useful for treating an inflammatory condition or for tissue repair or wound healing. See, e.g., Mimeault et al., Clin. Pharmacol. Therapeutics 82:252-64 (2007).

In some embodiments, the methods described herein are useful for mobilizing hematopoietic leukocytes (white blood cells) in a subject, which methods may be used in treating diseases, disorders, and conditions for which an increase in white blood cells, such as neutrophils, eosinophils, lymphocytes, monocytes, basophils, will provide clinical benefit. For example, for cancer patients, the compounds of Formula (I) are beneficial for stimulating neutrophil production to compensate for hematopoietic deficits resulting from chemotherapy or radiation therapy. Other diseases, disorders, and conditions to be treated include infectious diseases and related conditions, such as sepsis. When the subject to whom at least one compound of Formula (I) is administered is a donor, neutrophils may be collected for administration to a recipient subject who has reduced hematopoietic function, reduced immune function, reduced neutrophil count, reduced neutrophil mobilization, severe chronic neutropenia, leucopenia, thrombocytopenia, anemia, and acquired immune deficiency syndrome. Mobilization of mature white blood cells may be useful in subjects to improve or to enhance tissue repair, and to minimize or prevent vascular injury and tissue damage, for example following liver transplantation, myocardial infarction or limb ischemia. See, e.g., Pelus, Curr. Opin. Hematol. 15:285-92 (2008); Lemoli et al., Haematologica 93:321-24 (2008).

The compound of Formula (I) may be used in combination with one or more other agents that mobilize hematopoietic cells. Such agents include, for example, G-CSF; AMD3100 or other CXCR4 antagonists; GRO-β (CXCL2) and an N-terminal 4-amino truncated form (SB-251353); IL-8SDF-1α peptide analogs, CTCE-0021 and CTCE-0214; and the SDF1 analog, Met-SDF-10 (see, e.g., Pelus, supra and references cited therein). In some embodiments, a compound of Formula (I) may be administered with other mobilizing agents used in the art, which may permit administration of a lower dose of GCSF or AMD3100, for example, than required in the absence of a compound of Formula (I). The appropriate therapeutic regimen for administering a compound of Formula (I) in combination with another mobilizing agent or agents can be readily determined by a person skilled in the clinical art.

### Pharmaceutical Compositions and Methods of Using Pharmaceutical Compositions

Also provided herein are pharmaceutical compositions comprising at least one compound of Formula (I). In some embodiments, the pharmaceutical composition further comprises at least one pharmaceutically acceptable ingredient.

In pharmaceutical dosage forms, any one or more of the compounds of the present disclosure may be administered in the form of a pharmaceutically acceptable derivative, such as a salt, and/or it/they may also be used alone and/or in appropriate association, as well as in combination, with other pharmaceutically active compounds.

An effective amount or therapeutically effective amount refers to an amount of a compound of the present disclosure or a composition comprising at least one such compound that, when administered to a subject, either as a single dose or as part of a series of doses, is effective to produce at least one therapeutic effect. Optimal doses may generally be determined using experimental models and/or clinical trials. Design and execution of preclinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject. In general, the amount of at least one compound of Formula (I) as described herein, that is present in a dose, may range from about 0.01 μg to about 1000 μg per kg weight of the subject. The minimum dose that is sufficient to provide effective therapy may be used in some embodiments. Subjects may generally be monitored for therapeutic effectiveness using assays suitable for the disease or condition being treated or prevented, which assays will be familiar to those having ordinary skill in the art and are described herein. The level of a compound that is administered to a subject may be monitored by determining the level of the compound (or a metabolite of the compound) in a biological fluid, for example, in the blood, blood fraction (e.g., serum), and/or in the urine, and/or other biological sample from the subject. Any method practiced in the art to detect the compound, or metabolite thereof, may be used to measure the level of the compound during the course of a therapeutic regimen.

The dose of a compound described herein may depend upon the subject's condition, that is, stage of the disease, severity of symptoms caused by the disease, general health status, as well as age, gender, and weight, and other factors apparent to a person of ordinary skill in the medical art. Similarly, the dose of the therapeutic for treating a disease or disorder may be determined according to parameters understood by a person of ordinary skill in the medical art.

Pharmaceutical compositions may be administered in any manner appropriate to the disease or disorder to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the pharmaceutical composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical compositions described herein may be administered to a subject in need thereof by any one of several routes that effectively delivers an effective amount of the compound. Non-limiting suitable administrative routes include topical, oral, nasal, intrathecal, enteral, buccal, sublingual, transdermal, rectal, vaginal, intraocular, subconjunctival, sublingual, and parenteral administration, including subcutaneous, intravenous, intramuscular, intrasternal, intracavernous, intrameatal, and intraurethral injection and/or infusion.

The pharmaceutical composition described herein may be sterile aqueous or sterile non-aqueous solutions, suspensions or emulsions, and may additionally comprise at least one pharmaceutically acceptable excipient (i.e., a non-toxic material that does not interfere with the activity of the active ingredient). Such compositions may be in the form of a solid, liquid, or gas (aerosol). Alternatively, the compositions described herein may be formulated as a lyophilizate, or compounds described herein may be encapsulated within liposomes using technology known in the art. The pharmaceutical compositions may further comprise at least one additional component, which may be biologically active or inactive. Non-limiting examples of such components include buffers (e.g., neutral buffered saline or phosphate buffered saline), carbohydrates (e.g., glucose, mannose, sucrose or dextrans), mannitol, proteins, polypeptides, amino acids (e.g., glycine), antioxidants, chelating agents (e.g., EDTA and glutathione), stabilizers, dyes, flavoring agents, suspending agents, and preservatives.

Any suitable excipient or carrier known to those of ordinary skill in the art for use in pharmaceutical compositions may be employed in the compositions described herein. Excipients for therapeutic use are well known, and are described, for example, in Remington: The Science and Practice of Pharmacy (Gennaro, 21^{st} Ed. Mack Pub. Co., Easton, Pa. (2005)). In general, the type of excipient is selected based on the mode of administration, as well as the chemical composition of the active ingredient(s). Pharmaceutical compositions may be formulated for the particular mode of administration. For parenteral administration, pharmaceutical compositions may further comprise water, saline, alcohols, fats, waxes, and buffers. For oral administration, pharmaceutical compositions may further comprise at least one component chosen, for example, from any of the aforementioned excipients, solid excipients and carriers, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose, and magnesium carbonate.

The pharmaceutical compositions (e.g., for oral administration or delivery by injection) may be in the form of a liquid. A liquid pharmaceutical composition may include, for example, at least one the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. In some embodiments, the pharmaceutical composition comprises physiological saline. In some embodiments, the pharmaceutical composition an injectable pharmaceutical composition, and in some embodiments, the injectable pharmaceutical composition is sterile.

For oral formulations, at least one of the compounds of the present disclosure can be used alone or in combination with at least one additive appropriate to make tablets, powders, granules and/or capsules, for example, those chosen from conventional additives, disintegrators, lubricants, diluents, buffering agents, moistening agents, preservatives, coloring agents, and flavoring agents. The pharmaceutical compositions may be formulated to include at least one buffering agent, which may provide for protection of the active ingredient from low pH of the gastric environment and/or an enteric coating. A pharmaceutical composition may be formulated for oral delivery with at least one flavoring agent, e.g., in a liquid, solid or semi-solid formulation and/or with an enteric coating.

Oral formulations may be provided as gelatin capsules, which may contain the active compound or biological along with powdered carriers. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract.

A pharmaceutical composition may be formulated for sustained or slow release. Such compositions may generally be prepared using well known technology and administered by, for example, oral, rectal or subcutaneous implantation, or by implantation at the desired target site. Sustained-release formulations may contain the active therapeutic dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active therapeutic contained within a sustained release formulation depends upon the site of implantation, the rate and expected duration of release, and the nature of the condition to be treated or prevented.

The pharmaceutical compositions described herein can be formulated as suppositories by mixing with a variety of bases such as emulsifying bases or water-soluble bases. The pharmaceutical compositions may be prepared as aerosol formulations to be administered via inhalation. The compositions may be formulated into pressurized acceptable propellants such as dichlorodifluoromethane, propane, nitrogen and the like.

The compounds of the present disclosure and pharmaceutical compositions comprising these compounds may be administered topically (e.g., by transdermal administration). Topical formulations may be in the form of a transdermal patch, ointment, paste, lotion, cream, gel, and the like. Topical formulations may include one or more of a penetrating agent or enhancer (also call permeation enhancer), thickener, diluent, emulsifier, dispersing aid, or binder. Physical penetration enhancers include, for example, electrophoretic techniques such as iontophoresis, use of ultrasound (or “phonophoresis”), and the like. Chemical penetration enhancers are agents administered either prior to, with, or immediately following administration of the therapeutic, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneras et al., J. Pharm. Pharmacol. 54:499-508 (2002); Karande et al., Pharm. Res. 19:655-60 (2002); Vaddi et al., Int. J. Pharm. 91:1639-51 (2002); Ventura et al., J. Drug Target 9:379-93 (2001); Shokri et al., Int. J. Pharm. 228(1-2):99-107 (2001); Suzuki et al., Biol. Pharm. Bull. 24:698-700 (2001); Alberti et al., J. Control Release 71:319-27 (2001); Goldstein et al., Urology 57:301-5 (2001); Kiijavainen et al., Eur. J. Pharm. Sci. 10:97-102 (2000); and Tenjarla et al., Int. J. Pharm. 192:147-58 (1999).

Kits comprising unit doses of at least one compound of the present disclosure, for example in oral or injectable doses, are provided. Such kits may include a container comprising the unit dose, an informational package insert describing the use and attendant benefits of the therapeutic in treating the pathological condition of interest, and/or optionally an appliance or device for delivery of the at least one compound or composition comprising the same.

### EXAMPLES

### Example 1

### Synthesis of E-Selectin Inhibitor

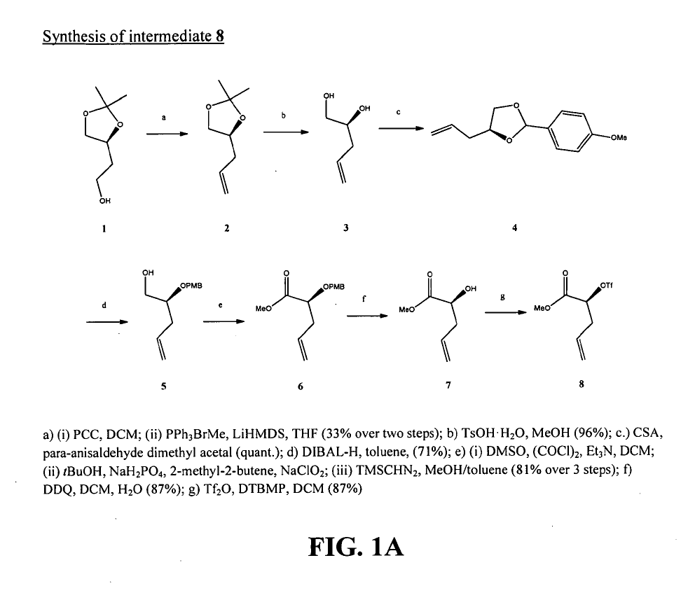

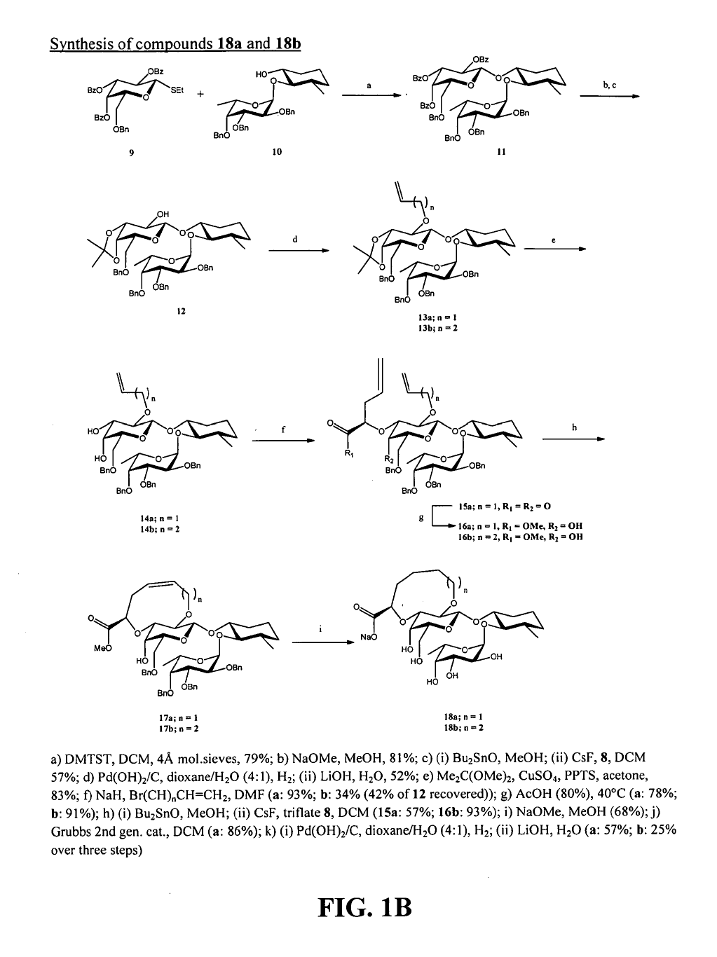

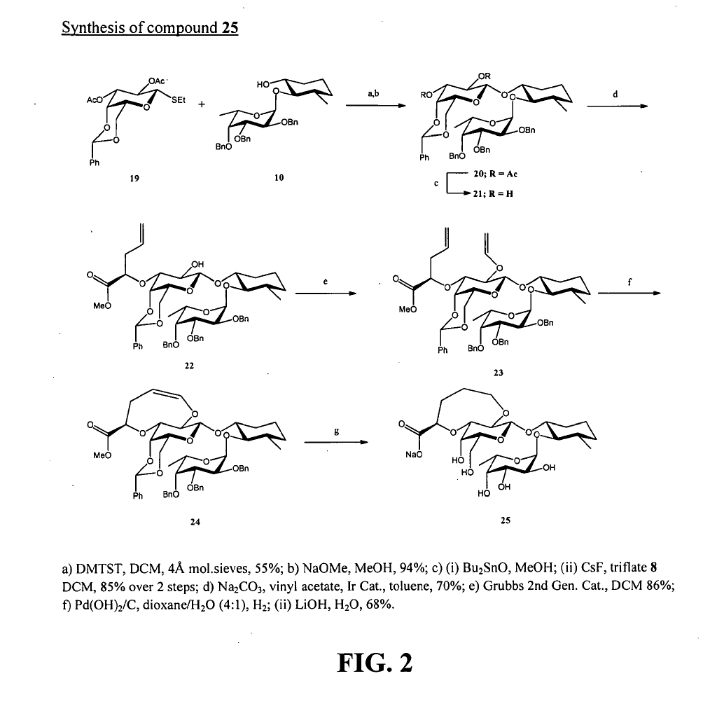

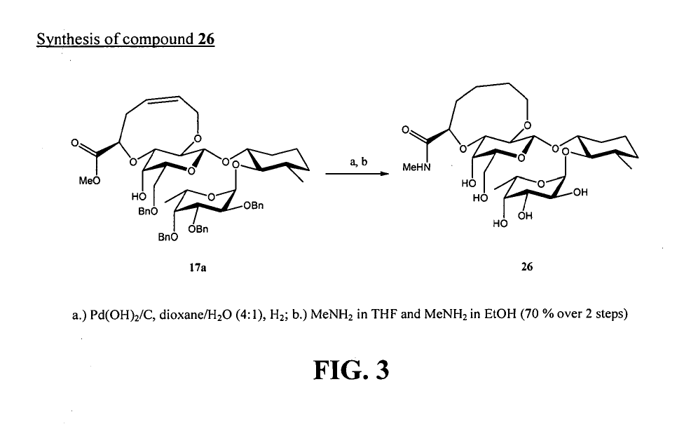

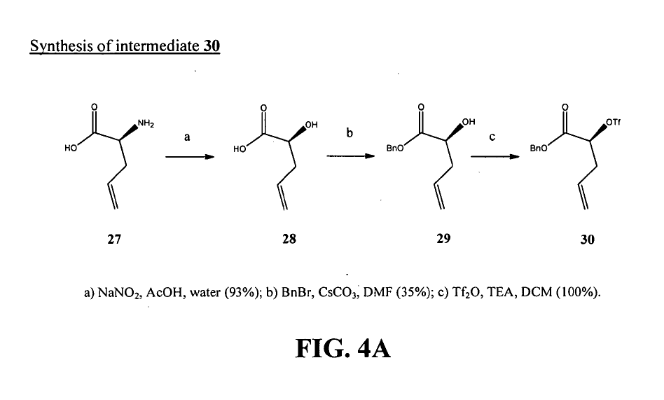

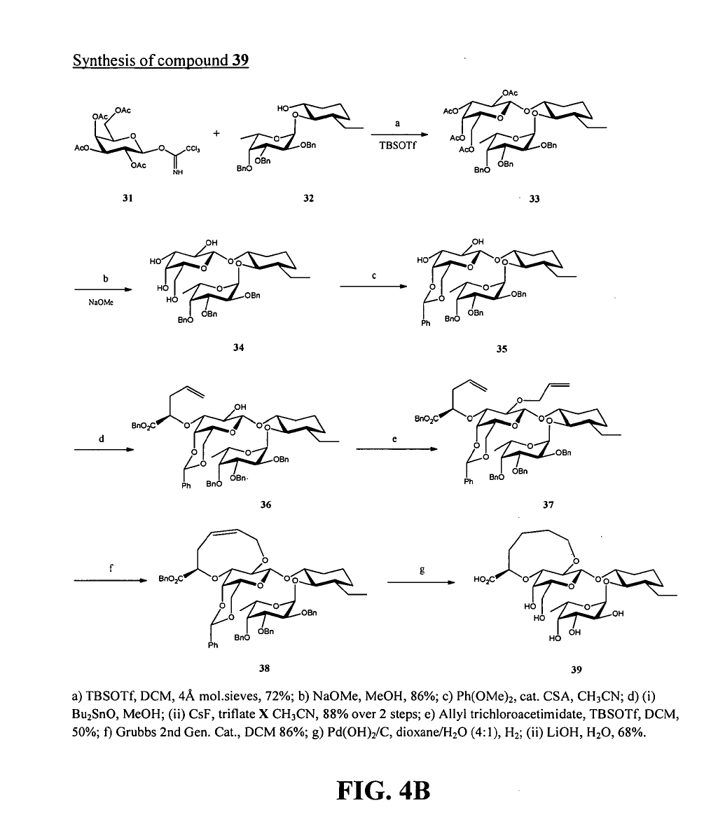

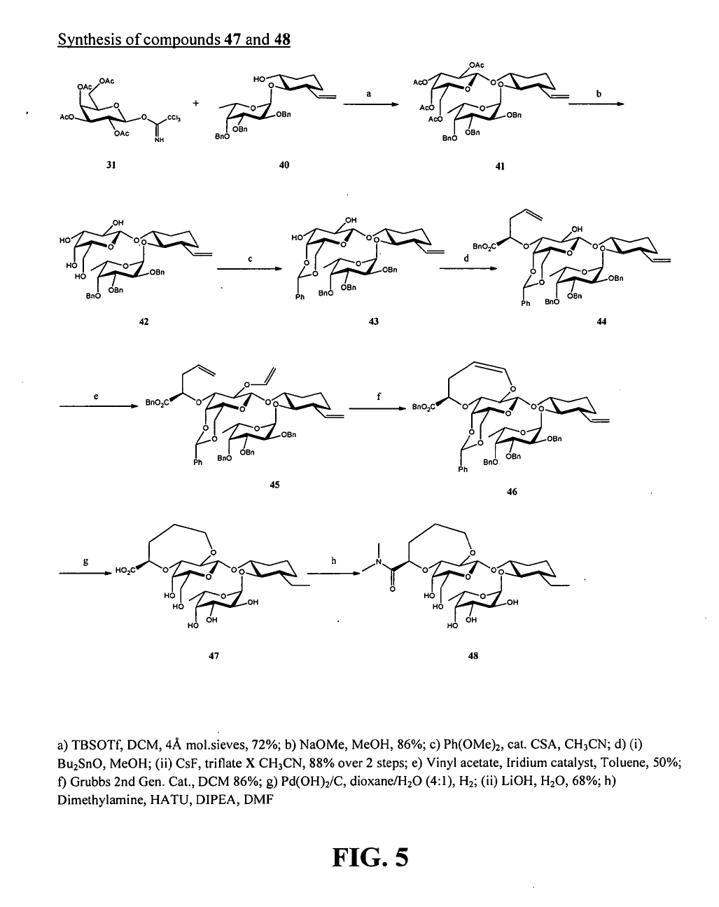

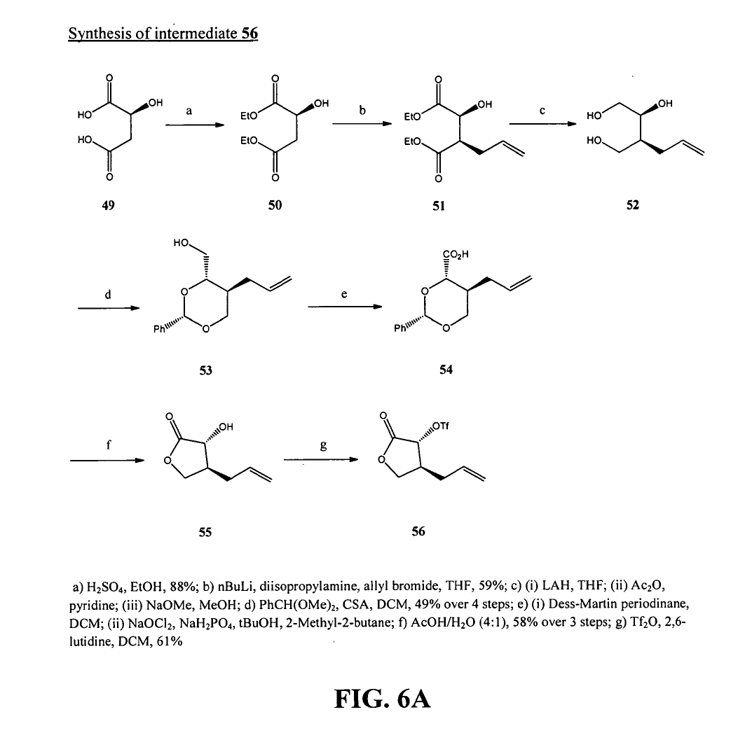

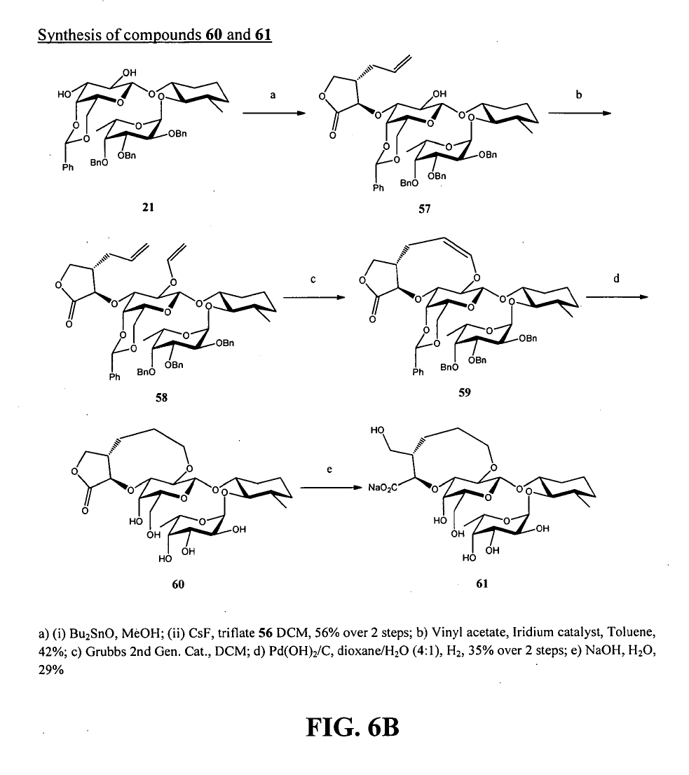

Exemplary glycomimetic compounds of Formula (I) were synthesized as described in this Example and as shown in the exemplary synthesis schemes set forth in FIGS. 1-6.

Synthesis of Compound 2:

(4S)-(+)-4-(2-Hydroxyethyl)-2,2-dimethyl-1,3-dioxolane 1 (1.00 g, 6.87 mmol) was dissolved in DCM (60 mL) and pyridinum chlorochromate (7.40 g, 34.4 mmol) was added at 0° C. The reaction mixture was poured into Et₂O (100 mL) and the resulting mixture was filtered through a pad of celite. The solvent was removed in vacuo and the crude aldehyde used without further purification in the next step.

Methyltriphenylphosphonium bromide (3.70 g, 10.3 mmol) was suspended in THF (30 mL) and LiHMDS (1.0 M solution in THF, 8.93 ml, 8.93 mmol) was added dropwise at −78° C. The mixture was stirred for 30 min at this temperature and additional 45 min at 0° C. The crude aldehyde of the previous step was added and the mixture was stirred at room temperature for 16 h. Water (30 mL) was added and the solution was washed with DCM (3×200 mL). The combined organic layers were dried (Na₂SO₄), filtrated and the solvent was removed in vacuo. The crude product was purified by flash chromatography (petroleum ether/EtOAc 20:1) to yield 2 as a colorless syrup (321 mg, 2.26 mmol, 33%). R_{f} (petroleum ether/EtOAc 10:1) 0.67; [α]_{D}²² 1.3 (c 4.90, CHCl₃); ¹H NMR (500.1 MHz, CDCl₃): δ=5.82-5.73 (m, 1H, R—H4), 5.13-5.05 (m, 2H, R—H5), 4.17-4.11 (m, 1H, R—H2), 4.01 (dd, J=6.0, 8.0, 1H, R—H1), 3.56 (dd, J=7.2, 7.9, 1H, R—H1′), 2.42-2.36 (m, 1H, R—H3), 2.30-2.23 (m, 1H, R—H3′), 1.40 (s, 1H, Me), 1.34 (s, 1H, Me). ¹³C NMR (125.8 MHz, CDCl₃): δ=133.8 (R—C4), 117.81 (R—C5), 109.2 (C(CH₃)₂), 75.4 (R—C2), 69.1 (R—C1), 38.2 (R—C3), 27.1 (Me), 25.8 (Me).

Synthesis of Compound 3:

Acetal 2 (673 mg, 4.73 mmol) was dissolved in MeOH (25 mL) and p-toluenesulfonic acid monohydrate (450 mg, 2.37 mmol) was added. The solution was stirred for 2.5 h at rt. The solvent was removed in vacuo and the crude product was purified by flash chromatography (DCM/MeOH 10:1) to yield 3 as colorless oil (465 mg, 4.55 mmol, 96%). R_{f} (petroleum ether/EtOAc 5:3) 0.14; [α]_{d}²² 11.4 (c 2.30, CHCl₃); ¹H NMR (500.1 MHz, CDCl₃): δ=5.82 (ddt, J=17.2, 10.2, 7.2 Hz, 1H, R—H5), 5.19-5.10 (m, 2H, R—H4), 3.81-3.73 (m, 1H, R—H2), 3.67 (dd, J=11.2, 3.1 Hz, 1H, R—H1′), 3.48 (dd, J=11.2, 7.2 Hz, 1H, R—H1), 2.30-2.18 (m, 2H, R—H3); ¹³C NMR (125.8 MHz, CDCl₃): δ=134.2 (R—C5), 118.4 (R—C4), 71.3 (R—C2), 66.4 (R—C1), 38.0 (R—C3).

Synthesis of Compound 4:

Diol 3 (465 mg, 4.55 mmol) was dissolved in DCM (25 mL) and camphorsulfonic acid and p-methoxybenzaldehyde dimethyl acetal (1.66 g, 9.11 mmol) were added. The solution was stirred at 50° C. for 15 h. The reaction was quenched with Et₃N and the solvent was removed in vacuo. The residue was purified by flash chromatography (petroleum ether/EtOAc 19:1) to give 4 as mixture of diastereomers (1.0 g, 4.55 mmol, quant.), which was used without further purification in the next step. R_{f} (petroleum ether/EtOAc 5:1) 0.75.

Synthesis of Compound 5:

Acetal 4 (1.00 g, 4.55 mmol) was dissolved in 50 ml toluene and DIBAL-H (1M in toluene, 9.1 mL) was added at 0° C. The reaction mixture was allowed to reach room temperature over 4 h and was filtered through a short pad of silica. The filtrat was evaporated and the crude product was purified by flash chromatography (petroleum ether/EtOAc 10:1 to 4:1) to yield 5 as a colorless liquid (0.72 g, 3.23 mmol, 71%). R_{f} (petroleum ether/EtOAc 5:1) 0.12; [α]_{d}²² 24.1 (c 1.40, CHCl₃); ¹H NMR (500.1 MHz, CDCl₃): δ=7.30-7.26 (m, 2H, Ar), 6.90-6.88 (m, 2H, Ar), 5.85-5.77 (m, 1H, R—H4), 5.15-5.05 (m, 2H, R—H5), 4.60 (d, J=11.7 Hz, 2H, Ph-CH₂), 4.47 (d, J=11.2 Hz, 1H, Ph-CH₂), 3.80 (s, 3H, Me), 3.68-3.65 (m, Hz, 1H, R—H1), 3.58-3.51 (m, 2H, R—H1′, R—H2), 2.42-2.37 (m, 1H, R—H3), 2.34-2.25 (m, 1H, R—H3′); ¹³C NMR (125.8 MHz, CDCl₃): δ=134.3 (R—C4), 128.9 (Ar), 117.7 (R—C5), 114.1 (Ar), 78.9 (R—C2), 71.4 (Ph-CH₂), 65.2 (R—C1), 55.5 (Me), 35.5 (R—C3).

Synthesis of Compound 6:

Oxalyl chloride (556 μl, 6.56 mmol) was dissolved in DCM (25 mL) and dimethylsulfoxid (622 μL, 8.75 mmol) was added at −78° C. and the mixture was stirred for 15 min at this temperature. A solution of alcohol 5 (487 mg, 2.19 mmol) DCM (5 mL) was added tropwise. The suspension was stirred for 35 min at −78° C., Et₃N (1.83 ml, 13.1 mmol) was added and the resulting solution was stirred for 1 h at rt. The reaction was quenched with water (80 mL) and the mixture was extracted with DCM (3×50 mL). The combined organic layers were dried, evaporated and used without further purification for the next step.

The crude aldehyde was dissolved in tBuOH (17 mL) and NaH₂PO₄ (420 mg, 3.5 mmol, in 2.8 mL H₂O), 2-methylbut-2-ene (40 mL, 2M in THF) and NaClO₂ (633 mg, 7.00 mmol) were added successively. The resulting solution was stirred for 2.5 h and the volatiles evaporated under reduced pressure. The residue was solved in DCM (60 mL) and the solution was dried (Na₂SO₄), concentrated under reduced pressure and used without further purification in the next step.

The crude acid was dissolved MeOH/toluene (1:2, 30 mL) and TMSCHN₂ (1.42 ml, 2 M in hexane) was added dropwise. The persistent of the yellow color indicated the end of the reaction. The solution was stirred for 1 h at rt, quenched with a few drops of Ac₂O and evaporated in vacuo. The crude ester was purified by flash chromatography (petroleum ether/EtOAc 6:1) to yield 6 (444 mg, 1.77 mmol, 81%) as a colorless liquid. R_{f} (petroleum ether/EtOAc 5:1) 0.60; [α]_{d}²²-78.8 (c 0.90, CHCl₃); ¹H NMR (500.1 MHz, CDCl₃): δ=7.27 (d, J=8.7 Hz, 2H, Ar), 6.87 (d, J=8.6 Hz, 2H, Ar), 5.80 (ddt, J=17.2, 10.2, 7.0 Hz, 1H, R—H4), 5.15-5.04 (m, 2H, R—H5), 4.63 (d, J=11.4 Hz, 1H, Ph-CH₂), 4.38 (d, J=11.4 Hz, 1H, Ph-CH₂), 4.02-3.96 (m, 1H, R—H2), 3.80 (s, 3H, Me), 3.74 (s, 3H, COOMe), 2.53-2.48 (m, 2H, H-3); ¹³C NMR (125.8 MHz, CDCl₃): δ=133.2 (R—C4), 129.9 (Ar), 129.8 (Ar), 129.6 (Ar), 118.1 (R—C5), 113.9 (Ar), 77.6 (R—C2), 72.12 (PhCH₂), 55.4 (Me), 52.0 (COOMe), 37.5 (R—C3).

Synthesis of Compound 7:

Ester 6 (100 mg, 400 μmol) was solved in DCM (9 mL) and H₂O (0.4 mL) and DDQ (95.3 mg, 420 μM) were added. The reaction mixture was stirred for 16 h at rt. The volatiles were removed under reduced pressure and the crude product was purified by flash chromatography (petroleum ether/EtOAc 5:1) to yield 7 (45.5 mg, 350 μmol, 87%) as a colorless liquid. R_{f} (petroleum ether/EtOAc 5:1) 0.27; [α]_{d}²² 10.5 (c 4.40, CHCl₃); ¹H NMR (500.1 MHz, CDCl₃): δ=5.80 (ddt, J=17.2, 10.2, 7.1 Hz, 1H, R—H4), 5.20-5.11 (m, 2H, R—H5), 4.28 (dd, J=6.3, 4.6 Hz, 1H, R—H2), 3.79 (s, 3H, COOMe), 2.62-2.54 (m, 1H, R—H3), 2.48-2.41 (m, 1H, R—H3′); ¹³C NMR (125.8 MHz, CDCl₃): δ=154.4 (COOMe), 132.7 (R—C4), 119.1 (R—C5), 70.2 (R—C2), 52.8 (Me), 38.9 (R—C3).

### Synthesis of Compound 8

Alcohol 7 (35.2 mg, 270 μmol) was dissolved in DCM (1.5 mL) and DTBMP (222 mg, 108 mM) and triflic anhydride (123 μL, 730 μmol) were added dropwise at −20° C.-−30° C. The resulting solution was stirred for 45 min at this temperature and further 45 min at 0° C. The reaction was diluted with DCM (50 mL) and washed with ice cold KH₂PO₄ (40 mL, 1M in H₂O). The aqueous layer was washed with DCM (2×50 mL). The organic phase was dried (Na₂SO₄) and the solvent removed in vacuo. The residue was purified by flash chromatography (petroleum ether/EtOAc 6:1) to yield triflate 8 (61.6 mg, 235 μmol, 87%) as a colorless liquid. R_{f} (petroleum ether/EtOAc 5:1) 0.61; [α]_{d}²²−14.1 (c 2.00, CHCl₃); ¹H NMR (500.1 MHz, CDCl₃): δ=5.80-5.69 (m, 1H, R—H4), 5.28-5.22 (m, 2H, R—H5), 5.16 (dd, J=7.5, 4.5 Hz, 1H, R—H2), 3.85 (s, 3H, Me), 2.83-2.68 (m, 2H, R—H3); ¹³C NMR (125.8 MHz, CDCl₃): δ=167.1 (COO), 129.5 (R—C4), 121.3 (R—C5), 123.85-112.31 (q, J=319.9 Hz, CF3), 82.6 (R—C2), 53.4 (Me), 36.4 (R—C3).

### Synthesis of Compound 11

Compound 10¹, see Schwizer et al., Chem.-Eur. J. 18:1342 (2012), (871 mg, 1.59 mmol) and thioglycoside 9 (1.10 g, 1.75 mmol) were dissolved in dry DCM (30 mL) and stirred together with powdered 4 Å activated molecular sieves (3 g) for 4 h at rt. DMTST (1.23 g, 4.77 mmol) was dissolved in DCM (10 mL) and stirred together with powdered 4 Å activated molecular sieves (1 g) for 3.5 h at rt as well. Both suspensions were combined and stirred for 3 d at rt. The mixture was filtered over a short pad of celite, washed with satd. aq. NaHCO₃ (40 mL) and water (40 mL). The combined aqueous phases were extracted with DCM (3×30 mL). The combined organic layers were dried (Na₂SO₄) and the solvent was removed in vacuo. The crude product was purified by flash chromatography (petroleum ether/EtOAc 4:1) to afford 11 (1.40 g; 1.26 mmol; 79%) as a white solid. Analytical data were in accordance with literature. See Binder, F., E- and P-selectin: differences and similarities guide the development of novel selectin antagonists, 2012 PhD Thesis, University of Basel, Switzerland.

### Synthesis of Compound 12

Benzoate 11 was suspended in a freshly prepared methanolic solution of NaOMe (12.6 mL, 0.02 M). The solution formed after a few minutes was stirred for 16 h at rt. The reaction mixture was neutralized with Dowex 50×8 ion exchange resin, filtered and concentrated in vacuo. The crude product was purified by flash chromatography to yield the intermediate triol as a white solid (815 mg, 1.02 mmol, 81%). Analytical data were in accordance with literature. See Binder, F., E- and P-selectin: differences and similarities guide the development of novel selectin antagonists, 2012 PhD Thesis, University of Basel, Switzerland.

The intermediate triol (810 mg, 1.01 mmol) was dissolved in acetone (50 mL) and 2,2-dimethoxypropane (497 μL, 4.06 mmol), CuSO₄ (2.43 g, 15.2 mmol) and PPTS (25.5 mg, 0.10 mmol) were added. The mixture was stirred for 16 h and additional 2,2-dimethoxypropane (248 μL, 505 μmol) and PPTS (50.9 mg, 0.20 mmol) were added. The suspension was stirred for 24 h and filtered over a short pad of Al₂O₃. The solvent was removed in vacuo and the crude product was purified by flash chromatography (petroleum ether/EtOAc 3:2) to yield 12 as a white solid (708 mg, 844 μmol, 83%). R_{f} (petroleum ether/EtOAc 1:1) 0.50; [α]d²²−47.4 (c 0.70, CHCl₃); ¹H NMR (500.1 MHz, CDCl₃): δ=7.35-7.21 (m, 20H, Ar—H), 5.08 (d, J=3.6 Hz, 1H, Fuc-H1), 4.95 (d, J=11.6 Hz, 1H, Ph-CH₂), 4.81 (d, J=11.7 Hz, 1H, Ph-CH₂), 4.74 (d, J=11.7 Hz, 2H, 2×Ph-CH₂), 4.67 (d, J=11.5 Hz, 1H, Ph-CH₂), 4.63-4.55 (m, 3H, 2×Ph-CH₂, Fuc-H5), 4.51 (d, J=11.9 Hz, 1H, Ph-CH₂), 4.24 (d, J=8.3 Hz, 1H, Gal-H1), 4.18 (dd, J=5.5, 2.1 Hz, 1H, Gal-H4), 4.09-4.00 (m, 3H, Fuc-H2, Gal-H3, Fuc-H3), 3.89 (td, J=6.2, 2.0 Hz, 1H, Gal-H5), 3.78 (dd, J=9.7, 6.2 Hz, 1H, Gal-H6), 3.76-3.68 (m, 2H, Gal-H6′, MeCy-H1), 3.65-3.63 (m, 1H, Fuc-H4), 3.46 (t, J=7.8 Hz, 1H, Gal-H2), 3.24 (t, J=9.4 Hz, 1H, MeCy-H2), 2.14-2.07 (m, 1H, MeCy-H6), 1.67-1.58 (m, 3H, MeCy-H3, MeCy-H5, MeCy-H4), 1.44 (s, 3H, Me), 1.36 (s, 3H, Me), 1.34-1.14 (m, 2H, MeCy-H6′, Cy-H5′), 1.11-1.07 (m, 6H, Fuc-H6, MeCy-Me), 1.06 0.95 (m, 1H, MeCy-H4′); ¹³C NMR (125.8 MHz, CDCl₃): δ=139.2, 139.0, 138.6, 138.3, 128.4-127.5 (24C, Ar—C), 110.1 (C(CH₃)₂), 99.5 (Gal-C1), 98.2 (Fuc-C1), 83.6 (MeCy-C2), 80.1 (Fuc-C3), 79.0 (Gal-C3), 78.8 (MeCy-C1), 78.2 (Fuc-C4), 76.5 (Fuc-C2), 74.9 (Ph-CH₂), 74.3 (Ph-CH₂), 73.7 (Ph-CH₂), 73.7 (Ph-CH₂), 73.5 (Gal-4), 72.9 (Gal-C2), 72.4 (Gal-C5), 69.5 (Gal-C6), 66.5 (Fuc-C5), 39.0 (MeCy-C3), 33.7 (MeCy-C4), 31.1 (MeCy-C6), 28.4 (Me), 26.4 (Me), 23.2 (MeCy-C5), 19.1 (MeCy-Me), 17.1 (Fuc-C6)); ESI-MS: m/z: Calcd for C₅₀H₆₂NaO₁₁ [M+Na]+: 861.42. found: 861.35.

### Synthesis of Compound 13a

Alcohol 15 (204 mg, 243 μmol) was dissolved in 8 mL DMF and sodium hydride (60% oil dispersion, 19.5 mg, 486 μmol) was added at 0° C. The mixture was stirred at this temperature for 1 h. The reaction was allowed to reach rt and allyl bromide (63.0 μL, 723 μmol) was added. The suspension was stirred for 16 h and diluted with MeOH (30 mL) and Et₂O (30 ml). The solution was extracted with H₂O (40 mL) and the aqueous phase was washed with Et₂O (2×40 mL). The combined organic layers were dried (Na₂SO₄), concentrated and the crude product was purified by flash chromatography (petroleum ether/EtOAc 3:1) to give 13a as a white foam (182 mg, 207 μmol, 85%). R_{f} (petroleum ether/EtOAc 2:1) 0.67; [α]_{d}²²−30.2 (c 1.20, CHCl₃); ¹H NMR (500.1 MHz, CDCl₃): δ=7.34-7.19 (m, 20H, Ar—H), 5.94-5.84 (m, 1H, CH₂═CH—CH₂), 5.32-5.25 (m, 1H, CH₂═CH), 5.15-5.10 (m, 1H, CH₂═CH), 5.10-5.07 (m, 1H, Fuc-H1), 4.95 (d, J=11.6 Hz, 1H, Ph-CH₂), 4.81 (d, J=11.7 Hz, 1H, Ph-CH₂), 4.79-4.75 (m, 1H, Ph-CH₂), 4.75-4.71 (m, 2H, 2×Ph-CH₂), 4.66 (d, J=11.4 Hz, 1H, Ph-CH₂), 4.62 (d, J=11.6 Hz, 1H, Ph-CH₂), 4.58 (d, J=12.0 Hz, 1H, Ph-CH₂), 4.48 (d, J=12.0 Hz, 1H, Ph-CH₂), 4.33-4.27 (m, 1H, CH₂═CH—CH₂), 4.26 (d, J=8.3 Hz, 1H, Gal-H1), 4.22-4.15 (m, 2H, CH₂═CH—CH₂, Gal-H4), 4.07-4.03 (m, 3H, Fuc-H2, Fuc-H3, Gal-H3), 3.85-3.79 (m, 1H, Gal-H5), 3.79-3.70 (m, 2H, Gal-H6, Gal-H6′), 3.68-3.65 (m, 1H, Fuc-H4), 3.64-3.58 (m, 1H, MeCy-H1), 3.27 (t, J=9.1 Hz, 1H, MeCy-H2), 3.22-3.16 (m, 1H, Gal-H2), 2.08-2.01 (m, 1H, MeCy-H6), 1.66-1.58 (m, 3H, MeCy-H3, MeCy-H4, MeCy-H5), 1.41 (s, 3H, Me), 1.35 (s, 3H, Me), 1.33-1.14 (m, 2H, MeCy-H5′, MeCy-H6′), 1.13-1.08 (m, 6H, MeCy-Me, Fuc-H6), 1.08-0.99 (m, 1H, MeCy-H4′); ¹³C NMR (125.8 MHz, CDCl₃): δ=¹³C NMR (125.8 MHz, CDCl₃) δ 139.2, 139.13, 138.78, 138.30, 128.72-127.3 (24×Ar—C), 135.2 (CH₂═CH—CH₂), 116.7 (CH₂═CH—CH₂), 109.7 (C(CH₃)₂), 101.3 (Gal-C1), 98.2 (Fuc-C1), 82.5 (MeCy-C2), 80.3 (Fuc-C3), 80.2 (Gal-C2), 80.0 (MeCy-C1), 79.4 (Gal-C3), 78.2 (Fuc-C4), 76.6 (Fuc-C2), 74.8 (Ph-CH₂), 74.3 (Ph-CH₂), 73.7 (Ph-CH₂), 73.7 (Gal-C4), 73.0 (Ph-CH₂), 72.8 (Ph-CH₂), 71.8 (Gal-H5), 69.3 (Gal-C6), 66.1 (Fuc-C5), 39.1 (MeCy-C3), 33.6 (MeCy-C4), 23.0 (MeCy-C5), 19.1 (Fuc-C6), 17.3 (MeCy-Me); ESI-MS: m/z: Calcd for C₅₃H₆₆NaO₁₁ [M+Na]+: 901.45. found: 901.47.

### Synthesis of Compound 13b

Alcohol 12 (716 mg, 854 μmol) was dissolved in DMF (8 mL) and sodium hydride (60% oil dispersion, 68.3 mg, 1.71 mmol) was added at 0° C. The mixture was stirred at this temperature for 1 h. The reaction was allowed to reach rt and pure but-3-en-1-yl triflate (375 mg, 1.84 mmol) was added. The suspension was stirred for 16 h and diluted with MeOH (100 mL) and Et₂O (100 mL). The solution was extracted with H₂O (80 mL) and the aqueous phase was washed with Et₂O (2×80 mL). The combined organic layers were dried (Na₂SO₄), concentrated and the crude product was purified by flash chromatography (petroleum ether/EtOAc 3:1) to give 13b as a white foam (258 mg, 289 μmol, 34% (58%)). Alcohol 12 was recovered in 42% yield (301 mg, 359 μmol). R_{f} (petroleum ether/EtOAc 1:1) 0.36; [α]_{d}²²−44.4 (c 0.50, CHCl₃); ¹H NMR (500.1 MHz, CDCl₃): δ=7.34-7.17 (m, 20H, Ar—H), 5.86-5.74 (m, 1H, CH₂═CH—CH₂), 5.10-5.03 (m, 2H, Fuc-H1, CH₂═CH—CH₂), 5.02-4.97 (m, 1H, CH₂═CH—CH₂), 4.94 (d, J=11.7 Hz, 1H, Ph-CH₂), 4.82-4.75 (m, 2H, Ph-CH₂, Fuc-H5), 4.72 (d, J=10.0 Hz, 2H, 2×Ph-CH₂), 4.66 (d, J=11.4 Hz, 1H, Ph-CH₂), 4.61 (d, J=11.7 Hz, 1H, Ph-CH₂), 4.57 (d, J=12.0 Hz, 1H, Ph-CH₂), 4.47 (d, J=12.0 Hz, 1H, Ph-CH₂), 4.22 (d, J=8.2 Hz, 1H, Gal-H1), 4.17-4.13 (m, 1H, Gal-H4), 4.07-4.04 (m, 2H, Fuc-H2, Fuc-H3), 4.03-3.99 (m, 1H, Gal-H3), 3.83-3.63 (m, 6H, 2×CH₂—CH₂—O, Gal-H5, Gal-H6, Gal-H6′, Fuc-H4), 3.63-3.56 (m, 1H, MeCy-H1), 3.25 (t, J=9.1 Hz, 1H, MeCy-H2), 3.13-3.08 (m, 1H, Gal-H2), 2.33-2.26 (m, 2H, CH₂═CH—CH₂), 2.05-1.99 (m, 1H, MeCy-H6), 1.65-1.54 (m, 3H, MeCy-H3, MeCy-H5, MeCy-H4), 1.41 (s, 3H, Me), 1.34 (s, 3H, Me), 1.31-1.12 (m, 2H, MeCy-H6′, MeCy-H5′), 1.12-0.99 (m, 7H, Fuc-H6, MeCy-Me, MeCy-H4); ¹³C NMR (125.8 MHz, CDCl₃): δ=139.2, 139.1, 138.8, 138.3, 128.6-127.3 (24C, Ar—C), 135.4 (CH₂═CH—CH₂), 116.4 (CH₂═CH—CH₂), 109.7 (Gal-C1), 101.1 (Fuc-C1), 98.1 (C(CH₃)₂), 82.3 (MeCy-C2), 81.1 (Gal-C2), 80.3 (Fuc-C3), 79.8 (MeCy-C1), 79.4 (Gal-C3), 78.2 (Fuc-C4), 76.6 (Fuc-C2), 74.8 (Ph-CH₂), 74.3 (Ph-CH₂), 73.7 (Ph-CH₂), 73.7 (Gal-C4), 72.8 (Ph-CH₂), 71.9 (CH₂—CH₂—O), 71.8 (Gal-C5), 69.4 (Gal-C6), 66.1 (Fuc-C5), 39.1 (MeCy-C3), 34.8 (CH₂═CH—CH₂), 33.6 (MeCy-C4), 31.0 (MeCy-C6), 28.3 (Me), 26.4 (Me), 21.0 (MeCy-C5), 19.1 (Fuc-C6), 17.3 (MeCy-Me); ESI-MS: m/z: Calcd for C₅₄H₆₈NaO₁₁ [M+Na]+: 915.47. found: 915.62.

### Synthesis of Compound 14a

Acetic acid (1 mL, 80%) was added to acetonide 13a (55.0 mg, 62.6 μmol) and the reaction mixture was stirred for 3 days at room temperature. The acetic acid was removed in vacuo and the residue was coevaporated with toluene. The crude product was purified by flash chromatography (petroleum ether/EtOAc 0 to 2:3) to yield 14a in (41.0 mg, 48.9 μmol, 78%) as a white solid. R_{f} (petroleum ether/EtOAc 2:1) 0.13; [α]_{d}²²−45.6 (c 3.64, CHCl₃); ¹H NMR (500.1 MHz, CDCl₃): δ=7.36-7.21 (m, 20H, Ar—H), 5.96-5.86 (m, 1H, CH₂═CH), 5.30-5.24 (m, 1H, CH₂═CH), 5.19-5.14 (m, 1H, CH₂═CH), 5.08 (d, J=3.1 Hz, 1H, Fuc-H1), 4.93 (d, J=11.5 Hz, 1H, PhCH₂), 4.87-4.79 (m, 2H, Fuc-H5, Ph-CH₂), 4.76-4.71 (m, 21-1, 2×Ph-CH₂), 4.66 (d, J=11.4 Hz, 1H, Ph-CH2), 4.58 (d, J=11.5 Hz, 1H, Ph-CH₂), 4.50 (s, 2H, 2×Ph-CH₂), 4.49-4.44 (m, 1H, CH₂═CH—CH₂), 4.35 (d, J=7.7 Hz, 1H, Gal-H1), 4.15-4.09 (m, 2H, Ph-CH₂, CH₂═CHCH₂), 4.08-4.02 (m, 3H, Fuc-H3, Gal-H4, Fuc-H2), 3.77 (dd, J=9.4, 6.9 Hz, 1H, Gal-H6), 3.73-3.71 (m, 1H, Fuc-H4), 3.70-3.59 (m, 2H, Gal-H6′, MeCy-C1), 3.57-3.51 (m, 2H, Gal-H3, Gal-H5), 3.34 (dd, J=9.2, 7.9 Hz, 1H, Gal-H2), 3.25 (t, J=9.2 Hz, 1H, MeCy-H2), 2.11-2.05 (m, 1H, MeCy-H6), 1.70-1.59 (m, 3H, MeCy-H3, MeCy-H4, MeCy-H5), 1.37-1.17 (m, 2H, MeCy-H6′, MeCy-H5′), 1.14 (d, J=6.5 Hz, 3H, Fuc-H6), 1.10 (d, J=6.5 Hz, 3H, MeCy-Me), 1.09-1.00 (m, 1H, MeCy-H4′); ¹³C NMR (125.8 MHz, CDCl₃): δ=139.3, 139.2, 138.7, 137.9, 128.8-127.3 (Ar—C), 135.0 (CH₂═CH), 117.3 (CH₂═CH), 101.4 (Gal-C1), 98.4 (Fuc-C1), 82.7 (MeCy-C2), 80.3 (Fuc-C3), 79.7 (MeCy-C1), 79.1 (Gal-C1), 78.5 (Fuc-C4), 76.6 (Fuc-C2), 75.1 (Ph-CH₂), 74.4 (Ph-CH₂), 73.8 (Ph-CH₂), 73.7 (Gal-C3), 73.5 (Gal-C5), 72.6 (Ph-CH₂), 69.1 (Gal-C6), 68.5 (Gal-C4), 66.3 (Fuc-C5), 39.2 (MeCy-C3), 33.6 (MeCy-C4), 31.1 (MeCy-C6), 23.1 (MeCy-C5), 19.0 (MeCy-Me), 17.2 (Fuc-C6); ESI-MS: m/z: Calcd for C₅₀H₆₂NaO₁₁ [M+Na]+: 861.42. found: 861.35.

### Synthesis of Compound 14b

Acetic acid (2 ml, 80%) was added to acetonide 13b (23.5 mg, 26.3 μmol) and the reaction mixture was stirred for 3 days at room temperature. The acetic acid was removed in vacuo and the residue was coevaporated with toluene. The crude product was purified by flash chromatography (petroleum ether/EtOAc 0 to 60%) to yield 14b in 91% (20.5 mg, 24.0 μmol). R_{f} (petroleum ether/EtOAc 2:1) 0.15; [α]_{d}²²−77.9 (c 0.40, CHCl₃); ¹H NMR (500.1 MHz, CDCl₃): δ=7.37-7.19 (m, 20H, Ar—H), 5.87-5.77 (m, 1H, CH₂═CH), 5.16-5.10 (m, 1H, CH₂═CH), 5.10-5.06 (m, 2H, CH₂═CH, Fuc-H1), 4.94 (d, J=11.5 Hz, 1H, Ph-CH₂), 4.88-4.79 (m, 2H, Fuc-H5, Ph-CH₂), 4.77-4.71 (m, 2H, 2×Ph-CH₂), 4.67 (d, J=11.4 Hz, 1H, Ph-CH₂), 4.59 (d, J=11.5 Hz, 1H, Ph-CH₂), 4.50 (s, 2H, 2×Ph-CH₂), 4.32 (d, J=7.7 Hz, 1H, Gal-H1), 4.09-3.98 (m, 4H, Fuc-H2, Fuc-H4, Gal-H4, CH₂═CH—CH₂), 3.77 (dd, J=9.4, 7.0 Hz, 1H, Gal-H6), 3.75-3.72 (m, 1H, Fuc-H4), 3.71-3.59 (m, 3H, Gal-H6′, MeCy-H2, CH₂═CH—CH₂), 3.56-3.49 (m, 2H, Gal-H5, Gal-H3), 3.31-3.22 (m, 2H, Gal-H2, MeCy-H2), 2.37-2.26 (m, 2H, O—CH₂—CH₂), 2.12-2.04 (m, 1H, MeCy-H6), 1.69-1.55 (m, 3H, MeCy-H5, MeCy-H3, MeCy-H4), 1.35-1.18 (m, 2H, MeCy-H6′, MeCy-H5′), 1.15 (d, J=6.5 Hz, 3H, Fuc-H6), 1.11 (d, J=6.5 Hz, 3H, MeCy-Me), 1.09-1.00 (m, 1H, MeCy-H4′); ¹³C NMR (125.8 MHz, CDCl₃): δ=139.3, 139.2, 138.7, 138.0, 128.8-127.3 (Ar—C), 136.0 (CH₂═CH), 117.1 (CH₂═CH), 101.3 (Gal-C1), 98.3 (Fuc-C1), 82.6 (MeCy-C2), 80.2 (Fuc-C3), 79.5 (MeCy-C1), 79.5 (Gal-C2), 78.5 (Fuc-C4), 76.5 (Fuc-C2), 75.1 (Ph-CH₂), 74.4 (Ph-CH₂), 73.7 (Ph-CH₂), 73.6 (Gal-C3), 72.7 (Gal-C5), 72.6 (Ph-CH₂), 71.8 (CH₂═CH—CH₂), 69.0 (Gal-C6), 68.2 (Gal-C4), 66.23 (Fuc-C5), 39.2 (Cy-C3), 34.8 (O—CH₂—CH₂), 33.6 (MeCy-C4), 31.1 (MeCy-C6), 23.1 (MeCy-C5), 19.0 (MeCy-Me), 17.2 (Fuc-C6); ESI-MS: m/z: Calcd for C₅₁H₆₄NaO₁₁ [M+Na]+: 875.43. found: 875.47.

### Synthesis of Compound 15a

Diol 14a (38.5 mg, 45.9 mmol) and Bu₂SnO (34.3 mg, 138 μmol) were dried for 16 h at rt, suspended in MeOH (3 mL) and refluxed for 2 h. The resulting solution was concentrated and the residue was coevaporated with toluene and the tin acetal was dried for 16 h under reduced pressure. The acetal was dissolved in freshly dried (Al₂O₃ column) DME (3 mL) and added to CsF (dried for 2 h at high vacuo at 100° C., 12.5 mg, 82.6 μmol). Triflate 8 (18.0 mg, 68.8 μmol) was added and the suspension was stirred for 16 hours at room temperature. A 10% solution of KF (in aq. 1M KH₂PO₄, 3 mL) was added. After stirring for 3 h at rt DCM (4 mL) was added and the aq. phase was extracted with DCM (2×10 ml). The combined organic layers were dried (Na₂SO₄) and concentrated under reduced pressure. Column chromatography on silica (petroleum ether/EtOAc 2:1) afforded 15a (25.0 mg, 26.3 μmol, 57%) as a white solid. R_{f} (petroleum ether/EtOAc 2:1) 0.49; [α]_{d}²²−39.2 (c 2.90, CHCl₃); ¹H NMR (500.1 MHz, CDCl₃): δ=7.39-7.19 (m, 20H, Ar—H), 5.94-5.83 (m, 2H, R—H4, O—CH₂—CH═CH₂), 5.35-5.29 (m, 1H, O—CH₂—CH═CH₂), 5.27-5.21 (m, 1H, R—H5), 5.21-5.16 (m, 2H, R—H5′, O—CH₂—CH═CH₂), 5.03 (d, J=3.7 Hz, 1H, Fuc-H1), 4.95 (d, J=11.2 Hz, 1H, Ph-CH₂), 4.83 (d, J=11.7 Hz, 1H, Ph-CH₂), 4.78-4.76 (m, 1H, Gal-H4), 4.76-4.70 (m, 3H, 2×Ph-CH₂, Fuc-H5), 4.67 (d, J=11.3 Hz, 1H, Ph-CH₂), 4.63 (d, J=11.3 Hz, 1H, Ph-CH₂), 4.51-4.43 (m, 3H, 2×Ph-CH₂, R—H2), 4.42-4.35 (m, 2H, Gal-H1, O—CH₂—CH═CH₂), 4.19 (dd, J=13.0, 6.4 Hz, 1H, O—CH₂—CH═CH₂), 4.05 (dd, J=10.3, 3.6 Hz, 1H, Fuc-H2), 3.99 (dd, J=10.3, 2.6 Hz, 1H, Fuc-H3), 3.90-3.85 (m, 2H, Gal-H3, Fuc-H4), 3.78-3.72 (m, 1H, Gal-H6), 3.68-3.58 (m, 3H, Gal-H6′, Gal-H5, Cy-H2), 3.43 (dd, J=9.9, 7.5 Hz, 1H, Gal-H2), 3.25 (t, J=9.4 Hz, 1H, Cy-H1), 2.74-2.66 (m, 2H, R—H3), 2.11-2.03 (m, 1H, Cy-H6), 1.68-1.56 (m, 3H, Cy-H5, Cy-H3, Cy-H4), 1.35-1.16 (m, 2H, Cy-H6′, Cy-H5′), 1.12 (d, J=6.5 Hz, 3H, Fuc-H6), 1.10 (d, J=6.4 Hz, 3H, Cy-Me), 1.04 (d, J=10.3 Hz, 1H, Cy-H4′), 1.08-0.99 (m, 1H, Cy-H4′); ¹³C NMR (125.8 MHz, CDCl₃): δ=168.6 (COO), 139.4, 139.1, 138.7, 137.7, 128.75-127.30 (24C, Ar—C), 135.0 (CH₂—CH═CH₂), 132.3 (R—C4), 119.6 (O—CH₂—CH═CH₂), 117.7 (R—C4), 101.8 (Gal-C1), 98.5 (Fuc-C1), 82.3 (Cy-C1), 80.2 (2C, Fuc-C3, Cy-C2), 78.7 (Fuc-C4), 76.0 (Fuc-C2), 75.5 (Ph-CH₂), 74.5 (Ph-CH₂), 74.4 (Gal-C4), 73.8 (Ph-CH₂), 73.4 (O—CH₂—CH═CH₂), 73.3 (Gal-C2), 72.7 (Gal-C3), 72.3 (Ph-CH₂), 71.4 (Gal-C5), 71.2 (R—C2), 66.6 (Gal-C6), 66.3 (Fuc-C5), 39.4 (Cy-C3), 37.5 (R—C3), 33.7 (Cy-C4), 31.1 (Cy-C6), 23.2 (Cy-C5), 19.0 (Fuc-C6), 16.9 (Cy-Me). ESI-MS: m/z: Calcd for C₅₆H₇₀NaO₁₃ [M+Na]+: 973.47. found: 973.48.

### Synthesis of Compound 16b

Alcohol 14b (24.5 mg, 28.7 mmol) and Bu₂SnO (21.4 mg, 86.2 μmol) were dried for 16 h at rt, suspended in MeOH (1.5 mL) and refluxed for 2 h. The resulting solution was concentrated and coevaporated with toluene and the tin acetal was dried for 16 h under reduces pressure. The acetal was dissolved in freshly dried (Al₂O₃ column) DME (1.5 mL) and added to CsF (dried for 2 h at high vacuo at 100° C., 7.85 mg, 51.7 μmol). Triflate 8 (11.3 mg, 43.1 μmol) was added and the suspension was stirred for 16 h at rt. A 20% solution of KF (in aq. 1M KH₂PO₄, 2 mL) was added. After stirring for 1 h at rt DCM (4 mL) was added and the aqueous phase was extracted with DCM (2×10 ml). The combined organic layers were dried (Na₂SO₄) and concentrated under reduced pressure. Column chromatography on silica (petroleum ether/EtOAc 2:1) afforded 16b (25.9 mg, 26.9 μmol, 93%) as a white solid. R_{f}(petroleum ether/EtOAc 2:1) 0.65; [α]_{d}²²−51.9 (c 0.60, CHCl₃); ¹H NMR (500.1 MHz, CDCl₃): δ=7.37-7.20 (m, 20H, Ar—H), 5.86-5.75 (m, 2H, R—H4, CH₂—CH₂—CH═CH₂), 5.21-5.14 (m, 2H, R—H5, R—H5′), 5.08-4.98 (m, 3H, 2×CH₂—CH₂—CH═CH₂, Fuc-H1), 4.94 (d, J=11.5 Hz, 1H, Ph-CH₂), 4.88-4.80 (m, 2H, Fuc-H5, Ph-CH₂), 4.75-4.67 (m, 3H, 3×Ph-CH₂), 4.60 (d, J=11.5 Hz, 1H, Ph-CH₂), 4.49 (s, 2H, 2×Ph-CH₂), 4.28 (d, J=7.2 Hz, 1H, Gal-H1), 4.24 (dd, J=7.9, 4.8 Hz, 1H, R—H2), 4.08-4.02 (m, 2H, Fuc-H2, Fuc-H3), 4.00-3.96 (m, 1H, Gal-H4), 3.90-3.84 (m, 1H, CH₂—CH₂—CH═CH2), 3.80-3.74 (n, 2H, Gal-H6, Fuc-H4), 3.73 (s, 3H, Me), 3.67-3.55 (m, 3H, Gal-H6′, MeCy-H1, CH2-CH₂CH═CH₂), 3.46-3.41 (m, 1H, Gal-H5), 3.38-3.31 (m, 2H, Gal-H2, Gal-H3), 3.24 (t, J=9.2 Hz, 1H, MeCy-H2), 2.58-2.43 (m, 2H, R—H3), 2.30-2.23 (m, 2H, 2×CH₂—CH₂—CH═CH₂), 2.07-2-01 (d, 1H, MeCy-H6), 1.70-1-53 (m, 3H, MeCy-H3, MeCy-H5, MeCy-H4), 1.32-1.18 (m, 2H, MeCy-H6′, MeCy-H5′), 1.15 (d, J=6.5 Hz, 3H, Fuc-H6), 1.12-0.97 (m, 4H, MeCy-Me, MeCy-H4′); ¹³C NMR (125.8 MHz, CDCl₃): δ=172.0 (COO), 139.4, 139.3, 138.8, 138.1, 128.8-127.2 (24C, Ar—C), 135.5 (CH₂—CH₂—CH═CH₂), 133.3 (R—C4), 119.1 (R—C5), 116.2 (CH₂—CH₂—CH═CH₂), 101.3 (Gal-C1), 98.3 (Fuc-C1), 82.3 (Cy-C2), 81.5 (Gal-C3), 80.3 (Fuc-C3), 79.4 (Cy-C1), 78.6 (Fuc-C4), 77.9 (Gal-C2), 76.5 (Fuc-C2), 75.1 (Ph-CH₂), 74.4 (Ph-CH₂), 73.8 (Ph-CH₂), 72.6 (Ph-CH₂), 72.2 (CH₂—CH₂—CH═CH₂), 72.1 (Gal-C5), 68.7 (Gal-C6), 66.4 (Gal-C4), 66.2 (Fuc-C5), 52.1 (Me), 39.2 (MeCy-C3), 37.9 (R—C3), 34.8 (CH₂—CH₂—CH═CH₂), 33.7 (MeCy-C4), 31.1 (MeCy-C6), 23.1 (MeCy-C5), 19.0 (MeCy-Me), 17.2 (Fuc-C6); ESI-MS: m/z: Calcd for C₅₇H₇₂NaO₁₃ [M+Na]+: 987.49. found 987.65.

### Synthesis of Compound 17a

Lactone 15a (79.1 mg, 83.2 μmol) was suspended in a freshly prepared methanolic solution of NaOMe (1.5 mL, 0.01 M). The solution formed after a few minutes, was stirred for 1.5 h at rt. The reaction mixture was quenched with a few drops of AcOH and concentrated in vacuo. The crude product was purified by flash chromatography to yield 16a as a white solid (53.6 mg, 56.4 mmol, 68%).

Methyl ester 16a (10.0 mg, 10.5 μmol) was dissolved in DCM (2 mL) and Grubbs 2^{nd} generation catalyst (0.89 mg, 1.05 μmol) was added and the solution stirred for 2 h at rt. The solvent was removed in vacuo and the crude product purified by flash chromatography (petroleum ether/EtOAc 3:1) to yield 17a (8.30 mg, 8.99 μmol, 86%) as a white solid. R_{f} (petroleum ether/EtOAc 3:1) 0.27; [α]_{d}²²−25.1 (c 0.57, CHCl₃); ¹H NMR (500.1 MHz, CDCl₃): δ=7.36-7.17 (m, 20H, Ar—H), 5.60-5.52 (m, 2H, R—H4, R—H5), 5.07 (d, J=2.6 Hz, 1H, Fuc-H1), 4.93-4.86 (m, 2H, Ph-CH₂, Fuc-H5), 4.82 (d, J=11.7 Hz, 1H, Ph-CH₂), 4.75-4.68 (m, 2H, 2×Ph-CH₂), 4.65 (d, J=11.4 Hz, 1H, Ph-CH₂), 4.59-4.46 (m, 4H, 3×Ph-CH₂, R—H5), 4.41 (dd, J=12.6, 2.5 Hz, 1H, R—H₂), 4.26 (d, J=7.7 Hz, 1H, Gal-H1), 4.20-4.11 (m, 2H, Gal-H4, R—H5′), 4.08-4.02 (m, 2H, Fuc-H2, Fuc-H3), 3.80-3.70 (m, 6H, Gal-H3, Me, Fuc-H4, Gal-H6), 3.68-3.60 (m, 2H, MeCy-H1, Gal-H6′), 3.55-3.46 (m, 2H, R—H3, Gal-H5), 3.24 (t, J=9.2 Hz, 1H, MeCy-H2), 3.18 (t, J=8.2 Hz, 1H, Gal-H2), 2.35-2.29 (m, 1H, R—H3′), 2.11-2.03 (m, 1H, MeCy-H6), 1.68-1.59 (m, 3H, MeCy-H3, MeCy-H4, MeCy-H5), 1.33-1.15 (m, 2H, MeCy-H5′, MeCy-H6′), 1.13 (d, J=6.5 Hz, 3H, Fuc-H6), 1.10 (d, J=6.5 Hz, 3H, MeCy-Me), 1.08-0.97 (m, 1H, MeCy-H4′); ¹³C NMR (125.8 MHz, CDCl₃): δ=172.5 (COO), 139.4, 139.3, 138.8, 138.2, 130.8, 128.8-127.3, 126.9 (26C, 24×Ar—C, CH═CH), 101.5 (Gal-C1), 98.3 (Fuc-C1), 82.5 (MeCy-C2), 80.5 (Gal-C2), 80.3 (Fuc-C3), 80.0 (MeCy-C1), 78.7 (Fuc-C4), 76.6 (R—C2), 76.5 (Fuc-C2), 76.2 (Gal-C3), 75.1 (Ph-CH₂), 74.4 (Ph-CH₂), 73.7 (Ph-CH₂), 72.6 (Gal-C5), 72.6 (Ph-CH₂), 71.9 (R—C5), 69.5 (Gal-C6), 69.2 (Gal-C4), 66.3 (Fuc-C5), 52.1 (Me), 39.1 (MeCy-C3), 33.7 (MeCy-C4), 31.6 (R—C3), 31.0 (MeCy-C6), 23.0 (MeCy-C5), 19.0 (MeCy-Me), 17.1 (Fuc-C6); ESI-MS: m/z: Calcd for C₅₄H₆₆NaO₁₃ [M+Na]+: 945.44. found: 945.47.

### Synthesis of Compound 18a

Compound 17a (12.2 mg, 13.2 μmol) was dissolved in dioxane/water (4:1, 1 mL) and Pd(OH)₂/C (1.5 mg, 10% Pd(OH)₂) was added. The suspension was stirred for 16 h under an atmosphere of hydrogen. Solvent was removed in vacuo.

The resulting ester was dissolved in H₂O (1 mL) and LiOH (0.63 mg, 26.4 μmol) was added. The turbid reaction mixture was stirred for 24 h at rt and the solvent was removed under reduced pressure. The residue was purified via RP chromatography (MeOH/H₂O), eluated through a sodium exchange column (Dowex 50/8 sodium form) and finally purified via size exclusion chromatography. Lyophilization from water/dioxane gave 18a (4.30 mg, 7.51 μmol), 57%) as a white fluffy foam. R_{f} (DCM/MeOH/H₂O 10:5:0.4) 0.30; [α]_{d}²²−68.1 (c 1.10, H₂O); ¹H NMR (500.1 MHz, D₂O) δ=5.13 (d, J=4.0 Hz, 1H, Fuc-H1), 4.94-4.89 (m, 1H, Fuc-H5), 4.49 (d, J=7.8 Hz, 1H, Gal-H1), 4.32-4.26 (m, 1H, R—H2), 4.18-4.15 (m, 1H, Gal-H5), 3.97-3.90 (m, 2H, Fuc-H3, R—H5), 3.84-3.78 (m, 3H, Fuc-H4, Fuc-H2, R—H5′), 3.77-3.65 (m, 4H, Gal-H6′, Gal-H6, Gal-H3, Cy-H1), 3.53 (t, J=5.9 Hz, 1H, Gal-H4), 3.35 (dd, J=9.2, 8.2 Hz, 1H, Gal-H2), 3.24 (t, J=9.4 Hz, 1H, Cy-H2), 2.20-2.09 (m, 3H, R—H4, R—H2, MeCy-H6), 1.86-1.73 (m, 3H, R—H2′, R—H3, R—H3′), 1.73-1.58 (m, 3H, MeCy-H5, MeCy-H4, MeCy-H3), 1.46-1.39 (m, 1H, R—H4), 1.37-1.24 (m, 2H, MeCy-H6′, MeCy-H5′), 1.22 (d, J=6.6 Hz, 3H, Fuc-H6), 1.11 (d, J=6.4 Hz, 4H, MeCy-Me, MeCy-H4′); ¹³C NMR (125.8 MHz, D₂O): δ=180.3 (COO), 100.1 (Gal-C1), 98.7 (Fuc-C1), 83.8 (MeCy-C2), 81.0 (R—C1), 79.0 (MeCy-C1), 77.6 (Gal-C2), 75.3 (Gal-C3), 74.6 (Gal-C4), 74.0 (R—C5), 72.0 (Fuc-C4), 69.3 (Fuc-C3), 69.0 (Gal-C5), 68.2 (Fuc-C2), 66.5 (Fuc-C5), 61.7 (Gal-C6), 38.5 (MeCy-C3), 33.1 (MeCy-C4), 30.4 (MeCy-C6), 29.0 (R—C2), 26.1 (R—C3), 25.3 (R—C4), 22.4 (MeCy-C5), 18.2 (MeCy-Me), 15.7 (Fuc-C6); ESI-MS: m/z: Calcd for C₂₅H₄₂NaO₁₃ [M+H]+: 573.2523. found: 573.2525.

### Synthesis of Compound 18b

Compound 16b (25.5 mg, 26.4 μmol) was dissolved in DCM (mL) and Grubbs 2^{nd} generation catalyst (4.49 mg, 5.28 μmol) was added. The solution was stirred for 2 h at rt. The solvent was removed in vacuo and the crude product purified by short column of silica (petroleum ether/EtOAc 3:1) to give 17b (20.4 mg) as a white solid.

3.7 mg of the olefin 17b (3.95 μmol) was dissolved in dioxane/water (4:1) and Pd(OH)₂/C (1 mg, 10% Pd(OH)₂) was added. The suspension was stirred for 16 h under an atmosphere of hydrogen. The solvent was removed under reduced pressure. The residue was dissolved H₂O (1 mL) and LiOH (0.19 mg, 7.90 μmol) was added. The resulting turbid reaction mixture was stirred for 24 h at rt. The solvent was removed under reduced pressure and the crude product was purified by RP chromatography (MeOH/H₂O), eluted through a sodium exchange column (Dowex 50/8 sodium form) and finally purified by size exclusion chromatography. Lyophilization from water/dioxane afforded 18b (800 μg, 1.42 μmol, 25%) as a white fluffy foam. R_{f} (DC M/MeOH/H₂O 10:5:0.4) 0.28; ¹H NMR (500.1 MHz, CDCl₃): δ=5.14 (d, J=4.0 Hz, 1H, Fuc-H1), 4.99-4.93 (m, 1H, Fuc-H5), 4.52 (d, J=7.3 Hz, 1H, Gal-H1), 4.40-4.35 (m, 1H, R—H2), 4.24-4.19 (m, 1H, R—H7), 4.11-4.08 (m, 1H, Gal-H5), 3.92 (dd, J=10.5, 3.3 Hz, 1H, Fuc-H3), 3.82 (d, J=22.7 Hz, 4H, Fuc-H2, Gal-H6, R—H7′, Fuc-H4), 3.70 (m, 4H, Gal-H2, Gal-H6′, MeCy-H1, Gal-H3), 3.54 (t, J=6.0 Hz, 1H, Gal-H4), 3.25 (t, J=9.5 Hz, 1H, MeCy-H2), 2.20-2.13 (m, 1H, MeCy-H6), 1.87-1.49 (m, 10H, MeCy-H3, MeCy-H5, MeCy-H4, R—H3, MeCy-H3′, MeCy-H4, MeCy-H4′, MeCy-H5, MeCy-H5′, MeCy-H6), 1.44-1.28 (m, 3H, MeCy-H6′, R—H6′, MeCy-H5′), 1.27 (d, J=6.6 Hz, 3H, Fuc-H6), 1.11 (d, J=6.4 Hz, 3H, MeCy-Me); ESI-MS: m/z: Calcd for C₂₆H₄₄NaO₁₃ [M+H]+: 587.2680. found: 587.2681.

### Synthesis of Compound 20

Compound 10 (300 mg, 549 μmol) and thioglycoside 19 (283 mg, 713 mmol) were dissolved in dry DCM (10 mL) and stirred together with 1 g powdered 4 Å activated molecular sieves for 4 h at rt. DMTST (425 mg, 1.65 mmol) was dissolved in DCM (5 mL) and stirred together with 0.5 g powdered 4 Å activated molecular sieves for 3.5 h at rt as well. Both suspensions were combined and stirred for 3 days at ambient temperature. The mixture was filtered through a short pad of celite, washed with an aq. sat. solution of NaHCO₃ (40 mL) and water (40 mL). The combined organic phases were extracted with DCM (3×30 mL). The combined organic layers were dried (Na₂SO₄) and the solvent was removed in vacuo. The crude product was purified by flash chromatography (petroleum ether/EtOAc 3:2) to yield 20 (266 mg, 302 μmol, 55%) as a white solid. Analytical data were in accordance with literature. See Binder, F., E- and P-selectin differences and similarities guide the development of novel selectin antagonists, 2012 PhD Thesis, University of Basel, Switzerland.

### Synthesis of Compound 21

Acetate 20 (156 mg, 178 μmol) was suspended in MeOH (5 mL) and sodium was added. The solution, formed after a few minutes was stirred for 16 h rt. The reaction mixture was neutralized by adding a few drops of glacial acetic acid and concentrated under reduced pressure. The crude product was purified by flash chromatography to yield 21 as a white solid (132 mg, 166 mot, 94%). Analytical data were in accordance with literature. See Binder, F., E- and P-selectin: differences and similarities guide the development of novel selectin antagonists, 2012 PhD Thesis, University of Basel, Switzerland.

### Synthesis of Compound 22

Alcohol 21 (60.0 mg, 75.3 μmol) and Bu₂SnO (56.2 mg, 226 μmol) were dried for 2.5 h at high vacuo, suspended in MeOH (4 mL) and refluxed for 2 h. The resulting solution was concentrated and coevaporated with toluene and the stannyl acetal was dried for 16 h at high vacuo. The residue was solved in DME (2 mL) and added together with triflate 8 (29.6 mg, 113 μmol) to CsF (dried for 2 h at high vacuo at 100° C., 20.6 mg, 136 μmol). The suspension was stirred for 16 h at rt. A 10% solution of KF (in 1M KH₂PO₄ solution, 3 mL) was added and the suspension was stirred for additional 1 h at rt. DCM (10 mL) was added and the aq. phase was separated. The aqueous phase was extracted with DCM (2×12 mL). The combined organic layers were dried (Na₂SO₄), concentrated under reduced pressure and purified by flash chromatography (petroleum ether/EtOAc 2:1) to afford 22 (58.4 mg, 64.2 μmol, 85%) as a white solid. R_{f} (petroleum ether/EtOAc 1:1) 0.35; [α]_{d}²²−18.5 (c 0.10, CHCl₃); ¹H NMR (500.1 MHz, CDCl₃): δ=7.61-7.14 (m, 20H, Ar—H), 6.00-5.85 (m, 1H, R—H4), 5.58 (s, 1H, Ph-CH), 5.17-5.11 (m, 1H, R—H5), 5.04-4.99 (m, 1H, R—H5′), 4.97 (d, J=3.4 Hz, 1H, Fuc-H1), 4.94-4.88 (m, 1H, Fuc-H5), 4.81 (d, J=11.7 Hz, 1H, Ph-CH₂), 4.70 (d, J=11.7 Hz, 1H, Ph-CH2), 4.60 (s, 2H, 2×Ph-CH₂), 4.37-4.31 (m, 2H, Gal-H6, Gal-H1), 4.25 (d, J=11.3 Hz, 1H, Ph-CH₂), 4.20-4.17 (m, 1H, Gal-H4), 4.14 (t, 1H, R—H2), 4.10-4.04 (m, 1H, Gal-H6′), 3.98-3.88 (m, 3H, Fuc-H3, Fuc-H2, Gal-H2), 3.76 (s, 3H, Me), 3.65-3.58 (m, 2H, MeCy-H1, Ph-CH₂), 3.41 (dd, J=9.6, 3.3 Hz, 1H, Gal-H3), 3.34-3.30 (m, 1H, Gal-H5), 3.28-3.20 (m, 2H, MeCy-H2, Fuc-H4), 2.59-2.53 (m, 2H, R—H3, R—H3′), 2.12-2.04 (m, 1H, MeCy-H6), 1.70-1.48 (m, 3H, MeCy-H5, MeCy-H3, MeCy-H4), 1.45-1.34 (m, 1H, MeCy-H6′), 1.29-1.15 (m, 1H, MeCy-H5′), 1.12-0.99 (m, 7H, Fuc-H6, MeCy-Me, Me-Cy-H4′); ¹³C NMR (125.8 MHz, CDCl₃): δ=173.2 (COO), 139.86, 139.68, 138.83, 138.25, 128.9-126.0 (24×Ar—C), 133.3 (R—C4), 118.3 (R—C5), 101.5 (Gal-C1), 99.8 (Ph-CH), 98.6 (Fuc-C1), 82.3 (MeCy-C2), 81.2 (Gal-C3), 80.3 (MeCy-C1), 79.9 (Fuc-C3), 78.9 (Fuc-C4), 77.2 (R—C2), 75.7 (Fuc-C2), 74.9 (Ph-CH₂), 74.6 (Ph-CH₂), 72.5 (Gal-C4), 71.4 (Ph-CH₂), 69.8 (Gal-C6), 68.9 (Gal-C2), 66.4 (Gal-C5), 66.2 (Fuc-C5), 52.5 (Me), 39.7 (MeCy-C3), 37.6 (R—C3), 33.9 (MeCy-C4), 31.5 (MeCy-C6), 23.5 (MeCy-C5), 19.0 (Fuc-H6), 16.7 (MeCy-Me); ESI-MS: m/z: Calcd for C₅₃H₆₄NaO₁₃ [M+Na]+: 931.42. found: 931.50.

### Synthesis of Compound 23

Alcohol 22 (14.1 mg, 15.5 μmol) and sodium carbonate (822 mg, 7.76 μmol) were dissolved in toluene (1 mL) and vinyl acetate (1 mL) and chloro(1,5-cyclooctadiene)iridium (I) dimer was added. The mixture was refluxed at 80° C. for 48 h. The reaction mixture was diluted with DCM (20 mL) and washed with satd. aq. NaHCO₃ (30 mL). The aqueous phase was washed with DCM (2×10 ml). The combined organic layers were dried (Na₂SO₄) and concentrated in vacuo. The crude product was purified by flash chromatography (0 to 50% petroleum ether/EtOAc+0.5% TEA) to yield 23 in 70% (10.1 mg, 10.8 μmol). R_{f} (petroleum ether/EtOAc 2:1) 0.38; [α]_{d}²²−4.4 (c 0.195, CHCl₃); ¹H NMR (500.1 MHz, CDCl₃): δ=7.61-7.56 (m, 2H, Ar—H), 7.37-7.13 (m, 18H, Ar—H), 6.38 (dd, J=13.7, 6.3 Hz, 1H, CH₂═CHO), 5.93-5.82 (m, 1H, R—H4), 5.59 (s, 1H, Ph-CH), 5.17-5.09 (m, 1H, R—H5), 5.06-5.01 (m, 1H, R—H5′), 4.99-4.91 (m, 2H, Fuc-H1, Fuc-H5), 4.81 (d, J=11.7 Hz, 1H, Ph-CH₂), 4.70 (d, J=11.7 Hz, 1H, Ph-CH₂), 4.64-4.57 (m, 2H, 2×Ph-CH₂), 4.37 (d, J=7.7 Hz, 1H, Gal-H1), 4.36-4.29 (m, 2H, CH₂═CHO, Gal-H6), 4.26-4.21 (m, 2H, Gal-H4, Ph-CH₂), 4.17 (dd, J=7.3, 5.6 Hz, 1H, R—H2), 4.11-4.04 (m, 1H, Gal-H6′), 3.99-3.86 (m, 4H, CH₂═CHO, Fuc-H3, Fuc-H2, Gal-H2), 3.70 (s, 3H, Me), 3.66 (dd, J=9.6, 3.5 Hz, 1H, Gal-H3), 3.61-3.54 (m, 2H, Ph-CH₂, MeCy-H1), 3.34-3.31 (m, 1H, Gal-H5), 3.26-3.20 (m, 2H, Fuc-H4, MeCy-H2), 2.58-2.49 (m, 2H, R—H3, R—H3′), 2.04-1.96 (m, 1H, MeCy-H6), 1.68-1.57 (m, 3H, MeCy-H3, MeCy-H5, MeCy-H4), 1.40-1.11 (m, 2H, MeCy-H6′, MeCy-H5′), 1.12-0.95 (m, 7H, MeCy-H4′, Fuc-H6, MeCy-Me); ¹³C NMR (125.8 MHz, CDCl₃): δ=153.8 (COO), 133.6 (R—C4), 129.0-125.9 (24×Ar—C), 118.1 (R—C5), 100.9 (Gal-C1), 99.9 (Ph-CH), 98.5 (Fuc-C1), 88.7 (CH₂═CHO), 82.0 (MeCy-C2), 81.5 (MeCy-C1), 79.8 (Fuc-C3), 78.9 (Gal-C3), 78.9 (Fuc-C4), 78.8 (Gal-C2), 77.9 (R—C2), 77.4 (Fuc-C2), 75.8 (Ph-CH₂), 75.0 (Ph-CH₂), 74.6 (Gal-C4), 73.7 (Ph-CH₂), 71.5 (Ph-CH₂), 69.6 (Gal-C6), 66.2 (Gal-C5), 66.1 (Fuc-C5), 52.1 (Me), 39.7 (MeCy-C3), 37.8 (R—C3), 37.8 (MeCy-C4), 31.2 (MeCy-C6), 23.5 (MeCy-C5), 19.9 (Fuc-C6), 16.8 (MeCy-Me); ESI-MS: m/z: Calcd for C₅₅H₆₆NaO₁₃ [M+Na]+: 957.44. found: 957.41.

### Synthesis of Compound 25

Compound 23 (4.2 mg, 4.49 μmol) was dissolved in DCM (1 mL) and Grubbs 2^{nd} generation catalyst (0.76 mg, 0.90 μmol) was added. The solution was stirred for 6 h at rt. Additional Grubbs 2^{nd} generation catalyst (0.76 mg, 0.90 μmol) was added and the solution stirred for another 10 h at ambient temperature. The solvent was removed under reduced pressure and the crude product purified by a short column of silica (petroleum ether/EtOAc 3:1+TEA). The olefin was dissolved in dioxane/water (4:1; 1 mL) and Pd(OH)₂/C (0.5 mg, 10% Pd(OH)₂) was added. The suspension was stirred for 16 h under an atmosphere of hydrogen, filtered and concentrated. The residue was solved in an aqueous LiOH solution (2 mL, 2.4 mM, 4.85 μmol) was added and the turbid mixture was stirred for 24 h at rt. The residue was purified via RP chromatography (MeOH/H₂O), eluated through a sodium exchange column (Dowex 50/8 sodium form) and finally purified via size exclusion chromatography. Lyophilization from water/dioxane gave 25 (1.46 mg, 2.62 μM, 58% over 3 steps) as a white fluffy foam. R_{f} (DCM/MeOH/H₂O 10:5:0.4) 0.30; [α]_{D}²²−74.7 (c 0.28, H₂O); ¹H NMR (500.1 MHz, D₂O): δ=5.13 (d, J=4.0 Hz, 1H, Fuc-H1), 4.89-4.75 (m, Fuc-H5), 4.62 (d, J=7.9 Hz, 1H, Gal-H1), 4.27 (dd, J=12.4, 2.3 Hz, 1H, R—H1), 4.23-4.19 (m, J=3.1 Hz, 1H, Gal-H4), 4.19-4.13 (m, 1H, R—H4), 4.02 (dd, J=9.7, 3.2 Hz, 1H, Gal-H3), 3.93 (dd, J=10.6, 3.3 Hz, 1H, Fuc-H3), 3.90-3.71 (m, 6H, R—H4′, Fuc-H4, Fuc-H2, MeCy-H1, Gal-H6, Gal-H6′), 3.62 (t, J=5.9 Hz, 1H, Gal-H5), 3.40 (dd, J=9.5, 8.0 Hz, 1H, Gal-H2), 3.27 (t, J=9.6 Hz, 1H, MeCy-H2), 2.20-2.06 (m, 2H, MeCy-H6, R—H2), 2.03-1.87 (m, 2H, R—H3, R—H2′), 1.73-1.55 (m, 4H, R—H3, MeCy-H4, MeCy-H5, MeCy-H3), 1.34-1.24 (m, 2H, MeCy-H5′, MeCy-H6′), 1.21 (d, J=6.6 Hz, 3H, Fuc-H6), 1.11 (d, J=6.3 Hz, 3H), 1.16-1.06 (m, MeCy-H4′); ¹³C NMR (125.8 MHz, D₂O): δ=99.6 (Fuc-C1), 98.5 (Gal-C1), 84.6 (MeCy-C2), 80.8 (R—C1), 79.9 (Gal-C2), 79.2 (Gal-C3), 79.2 (Gal-C4), 76.4 (Gal-C3), 75.9 (Gal-C5), 72.9 (Fuc-C4), 70.6 (R—C4), 70.2 (Fuc-C3), 69.1 (Fuc-C2), 67.3 (Fuc-C5), 62.5 (Gal-C6), 39.8 (MeCy-C3), 34.0 (Cy-C4), 30.9 (MeCy-C6), 28.0 (R—C3), 25.2 (R—C2), 23.5 (MeCy-C5), 19.2 (MeCy-Me), 16.5 (Fuc-C6); ESI-MS: m/z: Calcd for C₂₄H₄₀NaO₁₃ [M+H]+: 559.2367. found: 559.2365.

### Synthesis of Compound 26

Compound 17a (11.4 mg, 12.3 μmol) was dissolved in dioxane/water (4:1, 4 mL) and Pd(OH)₂/C (6 mg, 10% Pd(OH)₂) was added. The suspension was stirred for 16 h under an atmosphere of hydrogen. Solvent was removed in vacuo. The residue was purified via RP chromatography (MeOH/H₂O), eluated through a sodium exchange column (Dowex 50/8 sodium form) and finally purified via size exclusion chromatography. Lyophilization from water/dioxane gave methylester (4.90 mg, 868 μmol, 70%) as a white fluffy foam.

The crude product (3.90 mg, 1.06 μmol) was dissolved in MeNH₂ in THF (2M, 1 ml) and MeNH₂ in EtOH (8M, 1 ml) and stirred at rt for 24 h. The solvent was removed under reduced pressure and the crude product purified by RP flash chromatography and size exclusion chromatography to give 26 as a white solid (3.9 mg, 1.06 quant.). [α]_{d}²²−16.0 (c 0.28, H₂O); ¹H NMR (500.1 MHz, D₂O): δ=5.13 (d, J=3.2 Hz, 1H, Fuc-H1), 4.98-4.90 (m, 1H, Fuc-H5), 4.55-4.48 (m, 2H, R—H2, Gal-H1), 3.99-3.95 (m, 1H, Gal-H4), 3.94-3.87 (m, 2H, R—H6, Fuc-H3), 3.87-3.75 (m, 4H, R—H6′, Fuc-H2, Fuc-H4, Gal-H3), 3.74-3.62 (m, 3H, Cy-H1, Gal-H6, Gal-H6′), 3.59-3.48 (m, 2H, Gal-H2, Gal-H5), 3.24 (t, J=9.4 Hz, 1H, MeCy-H2), 2.77 (s, 3H, NH—CH₃), 2.18-2.10 (m, 1H, MeCy-H6), 2.00-1.84 (m, 2H, R—H3, R—H3′), 1.83-1.54 (m, 8H, MeCy-H3, MeCy-H4, MeCy-H5, R—H4, R—H4′, R—H5, R—H5′), 1.39-1.21 (m, 2H, MeCy-H6′, MeCy-H5′), 1.24 (d, J=6.4 Hz, 3H, Fuc-H6), 1.17-1.04 (m, 1H, Cy-H4′), 1.10 (d, J=6.2 Hz, 3H, MeCy-Me); ¹³C NMR (125.8 MHz, D₂O): δ=176.3 (COO), 100.5 (Gal-C1), 98.6 (Fuc-C1), 83.8 (Cy-C2), 79.2 (Cy-C1), 76.4 (Gal-C3), 75.5 (R—C2), 74.8 (Gal-C5), 73.8 (Gal-C2), 72.0 (Fuc-C4), 71.9 (Fuc-C2), 69.3 (Fuc-C3), 69.0 (R—C6), 68.2 (Gal-C4), 66.5 (Fuc-C5), 61.4 (Gal-C6), 38.5 (Cy-C3), 33.1 (Cy-C4), 30.5 (Cy-C6), 29.9 (R—C2), 25.5 (Me), 23.8 (R—C3), 22.4 (Cy-C5), 21.1 (R—C4), 18.2 (Cy-Me), 15.7 (Fuc-C6); HR-MS: m/z: Calcd for C₂₆H₄₅NNaO₁₂ [M+Na]+: 586.28. found: 586.31.

### Synthesis of Compound 28

A solution of (S)-2-aminopent-4-enoic acid (5.0 g, 43.4 mmole, 1.0 eq.) in water/acetic acid (100 mL, 8/2 v/v) is added a solution of NaNO₂ (7.5 g, 108.5 mmole, 2.5 eq.) in distilled water (20 mL) over 30 minutes period at 0° C. The resulting solution is stirred 2 hours at 0° C., then 12 hours at ambient temperature. The solution is then cooled down to 0° C. again, quenched with a solution of CH₃NH₂ in THF (2 M, 18.2 mL, 36.4 mmole, 2.0 eq.). THF is briefly evaporated under reduced pressure and the residual aqueous solution is acidified with conc. HCl to pH 2. This acidic solution is extracted with EtOAc (50 mL×4) and the organic layer is combined, dried over Na₂SO₄, and then concentrated under reduced pressure. The residue is purified by chromatography over silica gel with mixed solvent system of (CH₂Cl₂/MeOH/water, 10/5/0.5, v/v/v). The title compound is obtained in 4.7 g (40.4 mmole, 93%) as light brown gel. ES-MS; Calcd for C₅H₈O₃ [M−1]⁻, m/z 115.1. found 115.1.

### Synthesis of Compound 29

A solution of compound 8 (0.55 g, 4.73 mmole, 1.0 eq.) in anhydrous DMF (2 mL) is added Cs₂CO₃ (1.62 g, 4.97 mmole, 1.05 eq.) and the mixture is stirred for 15 minutes at ambient temperature. The mixture is cooled down to 0° C. and a solution of BnBr (0.59 mmole, 4.97 mmole, 1.05 eq.) in DMF (2 mL) is added drop wise over 30 minutes period. The resulting mixture is stirred for 1 hour at 0° C. then 12 hours at ambient temperature. The solution is concentrated under reduced pressure and the heterogeneous viscous residue is diluted with EtOAc (10 mL), washed with water, dried over Na₂SO₄ then concentrated. The crude product is purified by chromatography over silica gel with mixed solvent gradient from 5% to 50% EtOAc/Hexane. The title compound is obtained in 0.34 g (1.65 mmole, 35%) as yellow syrup. ES-MS; Calcd for C₁₂H₁₄O₃ [M+Na]⁺, m/z 229.0. found 229.1.

### Synthesis of Compound 30

A solution of compound 9 (0.27 g, 1.30 mmole, 1.0 eq.) and Et₃N (0.22 mL, 1.57 mmole, 1.2 eq.) in anhydrous CH₂Cl₂ (2 mL) is saturated with nitrogen then, cooled down to −40° C. Tf₂O (0.26 mL, 1.57 mmole, 1.2 eq.) is added drop wise over 5 minutes period and the resulting solution is stirred for 1 hour under the same condition. After completion of reaction, the solution is diluted with CH₂Cl₂ (10 mL), washed with distilled water, dried over Na₂SO₄, then concentrated. The crude product is purified by chromatography over silica gel with mixed solvent gradient from 5% to 20% EtOAc/Hexane. The title compound is obtained in 0.48 g (1.40 mmole, quantitative) as light brown syrup. R_{f}=0.69 (EtoAc/Hexane, 1/6, v/v).

### Synthesis of Compound 33

(1R,2R,3S)-3-ethyl-2-(((2S,3S,4R,5R,6S)-3,4,5-tris(benzyloxy)-6-methyltetrahydro-2H-pyran-2-yl)oxy)cyclohexan-1-ol (0.42 g, 0.75 mmole, 1.0 eq.) was dried by azeotroping twice with toluene (10 mL) followed by placement under high vacuum for 2 hours. A solution of acceptor in anhydrous dichloromethane (5 mL) was saturated with nitrogen and cooled to −40° C. To this solution was then added simultaneously via separate syringes a solution of commercially available compound 31 (0.72 g, 1.5 mmole, 2.0 eq.) in anhydrous CH₂Cl₂ (10 mL) and a solution of TBSOTf (0.17 mL, 0.75 mmole, 1.0 eq.) over a 35 minute period. The reaction was stirred for 2 hours under the same condition. Triethylamine (0.2 mL, 1.50 mmole, 2.0 eq.) was added slowly. The cooling bath was removed and the solution stirred for 10 minutes at room temperature. The solution was diluted with CH₂Cl₂ (50 mL), washed with water, brine, dried over Na₂SO₄, then concentrated under reduced pressure. The residue was purified by chromatography over silica gel with mixed solvent gradient from 10% EtOAC/Hexane to 50% EtOAc/Hexane to afford 0.48 g (0.54 mmole, 72% yield) compound 33 as a white foam. ES-MS; Calcd for C₄₉H₆₂O₁₅ [M+Na]⁺, m/z 913.4. found 913.4.

### Synthesis of Compound 34

A solution of compound 33 (0.9 g, 1.01 mmole, 1.0 eq.) in MeOH (10 mL) was cooled to 0° C. and a solution of NaOMe/MeOH (25 wt %) was added dropwise to get pH 9. The solution was stirred for 3 hours at 0° C. The solution was neutralized with H⁺ resin (Dowex HCR-W2, H⁺ form) to pH 7. The resin was filtered off and the filtrate was concentrated under reduced pressure. The crude product was purified by chromatography over silica gel with mixed solvent gradient from 100% EtOAc to 10% MeOH/EtOAc to afford 0.64 g (0.87 mmole, 86% yield) compound 34 as a white foam. ES-MS; Calcd for C₄₁H₅₄O₁₁ [M+Na]⁺, m/z 745.3. found 745.3.

### Synthesis of Compound 35

A solution of compound 34 (0.21 g, 0.29 mmole, 1.0 eq.) and benzaldehyde dimethyl acetal (0.13 mL, 0.87 mmole, 3.0 eq.) in anhydrous CH₃CN was heated to 60° C. A solution of camphosulfonic acid in CH₃CN was added over 2 hours period. The resulting solution was stirred another 3 hours under the same condition. After completion of reaction, the solution was cooled to room temperature and concentrated under reduced pressure. The residue was purified by chromatography over silica gel with EtOAc as an eluent to afford 0.13 g (0.16 mmole, 55% yield) compound 35 as a white solid. ES-MS; Calcd for C₄₈H₅₈O₁₂ [M+Na]⁺, m/z 833.4. found′833.3.

### Synthesis of Compound 36

A mixture of compound 35 (0.13 g, 0.16 mmole, 1.0 eq.) and dibutyltin oxide (52 mg, 0.2 mmole, 1.3 eq.) in anhydrous MeOH (5 mL) was saturated with nitrogen. The solution was stirred for 2 hours at 90° C. as it becomes homogeneous solution upon formation of tin complex. The solution was cooled to room temperature and concentrated under reduced pressure. The residue was co-evaporated with toluene (5 mL) three times and dried under high vacuum for 2 hours to dryness. This complex and dry cesium fluoride (49 mg, 0.32 mmole, 2.0 eq.) was dispersed in CH₃CN (3 mL) under nitrogen atmosphere at room temperature, then a solution of triflate 30 (0.11 g, 0.32 mmole, 2.0 eq.) in CH₃CN (3 mL) was added over 10 minutes. The resulting suspension was stirred overnight under the same condition. The suspension was diluted with EtOAc (10 mL), washed with water, brine, dried over Na₂SO₄, then concentrated under reduced pressure. The residue was purified by chromatography over silica gel with mixed solvent from 30% EtOAc/Hexane to 50% to afford 0.14 g (0.14 mmole, 88%) compound 36 as colorless sticky gel. ES-MS; Calcd for C₆₀H₇₀O₁₃ [M+Na]⁺, m/z 1021.4. found 1021.4.

### Synthesis of Compound 37

A solution of compound 36 (0.19 g, 0.19 mmole, 1.0 eq.) in anhydrous CH₂Cl₂ (4 mL) was saturated with nitrogen and cooled down to 0° C. A solution of allyl 2,2,2-trichloroacetimidate (0.77 g, 3.80 mmole, 20.0 eq.) in CH₂Cl₂ (4 mL) and a solution of TBSOTf in CH₂Cl₂ (1 mL) are simultaneously added drop wise over 30 minutes period. The resulting solution was stirred 12 hours under the same condition For completion of reaction, another 5 equivalent of imidate and 1 equivalent of activator are added in the same way as described above and the reaction was continued another 5 hours. Excess Et₃N (0.1 mL, 0.75 mmole) was added slowly and the solution was stirred for 15 minutes. The reaction was diluted with CH₂Cl₂ (10 mL), washed with water, brine, dried over Na₂SO₄ then concentrated. The residue was purified by chromatography over silica gel with mixed solvent gradient from 30% EtOAc/Hexane to 50% to afford 64 mg (0.06 mmole, 50% yield) compound 37 as white foam. ES-MS; Calcd for C₆₃H₇₄O₁₃ [M+H2O]⁺, m/z 1056.5. found 1056.4.

### Synthesis of Compound 38

A solution of compound 37 (64 mg, 60 μmole, 1.0 eq.) and benzylidene (2,5-dimesitylcyclopentyl)tricyclohexylphosphanylruthenium(V) chloride (Grubbs catalyst 2^{nd} generation, 10 mg, 12 μmole, 0.2 eq.) was saturated with nitrogen and the homogeneous brown solution was stirred 12 hours at ambient temperature. Another 0.1 eq. of catalyst (5 mg, 6 μmole, 1.0 eq.) was added and the reaction was continued additional 4 hours to complete the reaction. The solution was concentrated under reduced pressure and the residue was directly purified by chromatography over silica gel with mixed solvent gradient from 20% EtOAc/Hexane to 50% to afford 40 mg (40 μmole, 66%) compound 38 as a light brown foam. ES-MS; Calcd for C₆₁H₇₀O₁₃ [M+Na]⁺, m/z 1033.4. found 1033.4.

### Synthesis of Compound 39

A mixture of compound 38 (44 mg 43.5 μmole, 1.0 eq.) and Pd/C (wet, 10%, 40 mg, 100% by weight) in anhydrous EtOH (4 mL) was hydrogenated at 50 psi H₂ gas for 24 hours at ambient temperature. The solution was filtered through celite pad and the filtrate was concentrated under reduced pressure. The residue was run through on C-18 column using mixed solvent gradient from 100% water to 50% MeOH/H₂O. The product portion was collected, evaporated then lyophilized against distilled water to afford 10 mg (17.7 μmole, 41% yield) compound 39 as a white solid. H-1 NMR (DMSO-d₆, 600 MHz): δ=0.82 (t, J=7.40 Hz 3H), 0.84-0.88 (m, 1H), 0.95-1.07 (m, 1H), 1.04 (d, J=6.48 Hz, 3H), 1.10-1.19 (m, 1H), 1.21-1.36 (m, 3H), 1.37-1.48 (m, 2H)1.55-1.64 (m, 2H), 1.66-1.74 (m, 2H), 1.81-1.96 (m, 4H), 3.22 (m, 2H), 3.30-3.41 (m, partially hidden by DMSO signal, 3H), 3.47 (m, 1H), 3.48-3.3.57 (m, 3H), 3.58-3.62 (m, 4H), 3.96-4.02 (m, 2H), 4.14 (d, J=7.09 Hz, 1H), 4.16 (d, J=4.45 Hz, 1H), 4.20 (d, J=7.27 Hz, 1H), 4.24 (d, J=6.00 Hz, 1H), 4.46 (m, 1H), 4.55 (m, 1H), 4.67 (d, J=3.78 Hz, 1H). ES-MS; Calcd for C₂₆H₄₄O₁₃ [M−1]⁻, m/z 563.2. found 563.3.

### Synthesis of Compound 41

(1R,2R,3R)-2-(((2S,3S,4R,5R,6S)-3,4,5-tris(benzyloxy)-6-methyltetrahydro-2H-pyran-2-yl)oxy)-3-vinylcyclohexan-1-ol (Compound 40) (4.80 g, 8.60 mmole, 1.0 eq.) was dried by azeotroping twice with toluene (30 mL) followed by high vacuum for 2 hours. Compound 40, described in U.S. Pat. No. 7,964,569, was dissolved in anhydrous dichloromethane (120 mL) and cooled to −40° C. To this solution was then simultaneously added a solution of compound 31 (6.20 g, 12.9 mmole, 1.5 eq.) in anhydrous CH₂Cl₂ (50 mL) and a solution of TBSOTf (2.0 mL, 8.60 mmole, 1.0 eq.) over a 1 hour period. The reaction mixture was stirred for 6.5 hours under the same condition. Triethylamine (2.4 mL, 17.2 mmole, 2.0 eq.) was added slowly and the solution was allowed to stir for 20 minutes while temperature was raised to room temperature. The solution was diluted with CH₂Cl₂ (100 mL), washed with water, brine, dried over Na₂SO₄, then concentrated under reduced pressure. The residue was purified by chromatography over silica gel with mixed solvent gradient from 10% EtOAC/Hexane to 50% to afford 5.4 g compound 41 (6.07 mmole, 70% yield) as a colorless sticky gel. ES-MS; Calcd for C₄₉H₆₀O₁₅Na [M+Na]⁺, m/z 911.4. found 911.3.

### Synthesis of Compound 42

A solution of compound 41 (2.6 g, 2.92 mmole, 1.0 eq.) in MeOH (60 mL) was cooled to 0° C. and a solution of NaOMe/MeOH (25 wt %) was added dropwise to get pH 9. The reaction mixture was stirred for 1.5 hours then concentrated under reduced pressure. The crude product was purified by chromatography over silica gel with mixed solvent gradient from 100% EtOAc to 10% MeOH/EtOAc to afford 1.9 g (2.63 mmole, 90% yield) compound 42 as a white solid. ES-MS; Calcd for C₄₁H₅₂O₁₁Na [M+Na]⁺, m/z 743.3. found 743.3.

### Synthesis of Compound 43

A solution of compound 42 (0.29 g, 0.40 mmole, 1.0 eq.) and benzaldehyde dimethyl acetal (0.09 mL, 0.60 mmole, 1.5 eq.) in anhydrous CH₃CN (3.5 mL) was heated to 60° C. prior to addition of acid catalyst. A solution of camphosulfonic acid (9.3 mg, 0.04 mmole, 0.1 eq.) in CH₃CN (1.5 mL) was then added over a 2 hour period while the solution is stirred. The resulting solution was stirred another 1.5 hours under the same condition. The solution was cooled to room temperature and concentrated under reduced pressure. The residue was purified by chromatography over silica gel with EtOAc as an eluent to afford 0.22 g (0.27 mmole, 68% yield) of compound 43 as a white foam. ES-MS; Calcd for C₄₈H₅₆O₁₁Na [M+Na]⁺, m/z 808.3. found 831.3.

### Synthesis of Compound 44

A mixture of compound 43 (1.4 g, 1.73 mmole, 1.0 eq.) and dibutyltin oxide (0.56 g, 2.25 mmole, 1.3 eq.) in anhydrous MeOH (50 mL) was stirred for 2 hours at 90° C. during which time it became homogenous. The solution was cooled to room temperature and concentrated under reduced pressure. The residue was co-evaporated with toluene (30 mL) three times and dried under high vacuum for 2 hours. This complex and dry cesium fluoride (0.53 g, 3.46 mmole, 2.0 eq.) were dispersed in CH₃CN (30 mL) under nitrogen atmosphere at room temperature, then a solution of triflate 30 (1.17 g, 3.46 mmole, 2.0 eq.) in CH₃CN (20 mL) was added over 1.5 hours period. The resulting suspension was stirred overnight under the same condition. The reaction mixture was diluted with EtOAc (100 mL), washed with water, brine, dried over Na₂SO₄, then concentrated under reduced pressure. The residue was purified by chromatography over silica gel with mixed solvent from 30% EtOAc/Hexane to 50% to afford 1.48 g of compound 44 (1.48 mmole, 86% yield) as a white foam. ES-MS; Calcd for C₆₀H₆₈O₁₃Na [M+Na]⁺, m/z 1019.4. found 1019.4.

### Synthesis of Compound 45

To a solution of compound 44 (0.13 g, 0.13 mmole, 1.0 eq.) and Na₂CO₃ (7.0 mg, 0.065 mmole, 0.5 eq.) in anhydrous toluene/vinyl acetate (1/1 v/v, 20 mL) under a nitrogen atmosphere was added [Ir(COD)Cl]₂ (45 mg, 0.065 mmole, 0.5 eq.). The resulting solution was stirred 24 hours under the same condition. The reaction was diluted with EtOAc (50 mL), washed with saturated NaHCO₃, water, brine, dried over Na₂SO₄ then concentrated. The residue was purified by chromatography over silica gel with mixed solvent gradient from 25% EtOAc/Hexane to 50% to afford 0.12 g of compound 45 (0.11 mmole, 85% yield) as a light brown foam. ES-MS; Calcd for C₆₂H₇₄O₁₄ [M+H₂O]⁺, m/z 1042.5. found 1042.5.

### Synthesis of Compound 46

A solution of compound 45 (0.27 g, 0.26 mmole, 1.0 eq.) and benzylidene(2,5-dimesitylcyclopentyl)tricyclohexylphosphanylruthenium (V) chloride (Grubbs catalyst 2″ generation, 0.22 g, 0.26 mmole, 1.0 eq.) was saturated with nitrogen and the homogeneous brown solution was stirred 2.5 hours at ambient temperature. The reaction mixture was concentrated under reduced pressure. The residue was purified by chromatography over silica gel with mixed solvent gradient from 20% EtOAc/Hexane to 50% to afford 70 mg compound 46 (0.07 mmole, 35% yield) as a light brown solid. ES-MS; Calcd for C₆₀H₆₆O₁₃Na[M+Na]⁺, m/z 1017.4. found 1017.3.

### Synthesis of Compound 47

A mixture of compound 46 (0.14 g, 0.14 mmole, 1.0 eq.) and Pd(OH)₂ (70 mg) in a solution of 1,4-dioxane/water (4/1 v/v, 10 mL) was stirred under an atmosphere of H₂ overnight at ambient temperature. The solution was filtered through celite and the filtrate was concentrated under reduced pressure. The residue was purified by chromatography over silica gel with a solution of EtOAc/MeOH/water (10/6/1, v/v/v) as an eluent. The product portion is collected, evaporated then lyophilized from distilled water to afford 75 mg compound 47 (0.13 mmole, 74% yield) as a white solid. H-1 NMR (D₂O, 600 MHz): δ=0.73 (m, 3H), 0.82-0.89 (m, 1H), 1.05 (d, 3H), 1.09-1.16 (m, 3H), 1.32-1.33 (m, 1H), 1.53-1.67 (m, 3H), 1.68-1.76 (m, 1H), 1.78-1.88 (m, 2H), 1.1.92-2.04 (m, 3H), 3.20-3.29 (m, 2H), 3.44-3.47 (m, 1H), 3.50-3.74 (m, 5H), 3.75-3.79 (dd, 1H), 3.86-3.93 (dd, 1H), 3.98-4.03 (m, 1H), 4.05-4.12 (br d, 1H), 4.21-4.27 (m, 1H), 4.46 (d, 1H), 4.66-4.71 (br q, 1H), 4.93 (d, 1H). ES-MS; Calcd for C₂₅H₄₁O₁₃ [M−1]⁻, m/z 550.2. found 549.3.

### Synthesis of Compound 48

To a solution of compound 47 (40 mg, 72 μmole, 1.0 eq.) and N,N-diisopropylethylamine (50 μL, 0.29 mmole, 4.0 eq.) in anhydrous DMF (1 mL) cooled to 0° C. was added HATU (41 mg, 0.11 mmole, 1.5 eq.). The resulting solution was stirred 5 minutes. A solution of Me₂NH in THF (2M, 1 mL, 2.0 mmole, 27 eq.) was slowly added. The resulting solution was stirred 50 minutes under the same condition. The solution was concentrated under reduced pressure. The residue was purified by chromatography over silica gel with a solution of (EtOAc/MeOH/water, 10/5/0.5, v/v/v) followed by C-18 column chromatography with gradient solution of water/MeOH (100% water to 50% water in MeOH) to afford 21 mg of compound 48 (36 μmole, 50% yield) as a white solid. H-1 NMR (D₂O, 600 MHz): δ=0.73 (t, 3H), 0.81-0.90 (m, 1H), 1.05 (d, 3H), 1.07-1.1.19 (m, 3H), 1.29-1.36 (m, 1H), 1.43-1.49 (m, 1H), 1.53-1.66 (m, 3H), 1.67-1.74 (m, 1H), 1.82-1.90 (m, 1H), 1.99 (m, 1H), 2.06-2.14 (m, 1H), 2.79 (s, 3H), 3.03 (s, 3H), 3.20-3.26 (m, 2H), 3.42 (m, 1H), 3.53 (dd, 1H), 3.54-3.60 (m, 1H), 3.62-3.68 (m, 4H), 3.69-3.73 (ddd, 1H), 3.76 (dd, 1H), 3.83 (dd, 1H), 4.03 (m, 1H), 4.47 (d, 1H), 4.68 (m, 1H), 4.72 (br d, 1H), 4.93 (d, 1H). ES-MS; Calcd for C₂₇H47O₁₂ [M+Na]⁻, m/z 600.3. found 600.2.

### Synthesis of Compound 50

(5)-Maleic acid (49, 10.0 g) was dissolved in EtOH (80 mL) and H₂SO₄ (0.25 mL) was added. The solution was refluxed for 20 h, quenched with Et₃N (0.5 mL) and the solvent was removed under reduced pressure. The residue was purified by flash chromatography (petroleum ether/EtOAc, 10:1 to 1:1) to yield 2 (12.5 g, 65.9 mmol, 88%) as colorless syrup. The analytical data were in accordance with P. Wipf et al., Chem.-Eur. J. 8:1670-1681 (2002).

### Synthesis of Compound 51

Experimental procedure (yield: 59%) and analytical data were in accordance with Khan et al., Eur. J. Org. Chem. 2012:995-1002 (2012).

### Synthesis of compound 53

A solution of 51 (100 mg, 435 μmol) in THF (1.5 mL) was added to a solution of LiAlH₄ (1 M, 956 μL, 956 μmol) in THF. The mixture was stirred for 2 h at rt and quenched with AcOH (80 μL) at 0° C. Pyridine (2 mL) and Ac₂O (1 mL) were added and the reaction mixture stirred at 80° C. for 16 h. The mixture was diluted with 1 M aq. hydrochloric acid (10 mL) and extracted with Et₂O (2×15 mL). The combined organic layers were washed with satd. aq. NaHCO₃ (3×10 mL). The organic layer was dried (Na₂SO₄), the solvents were removed in vacuo and the residue coevaporated with toluene. The crude residue was treated with NaOMe/MeOH (0.02 M, 2.5 mL) for 3 h at rt. The reaction was quenched with 2 drops of AcOH and evaporated in vacuo to give intermediate 52 as yellow oil, which was used without further purification in the next step.

Triol 52 was dissolved in DCM (1.5 mL) and benzaldehyde dimethyl acetal (72.7 mg, 478 μmol, 71.7 μL) and camphorsulfonic acid (20.2 mg, 86.9 μmol) were added. The solution was stirred at 50° C. for 16 h, then quenched with Et₃N and concentrated in vacuo. The residue was purified by flash chromatography (petroleum ether/EtOAc, 3:1) to give 53 (49.6 mg, 212 μmol, 49%) as colorless oil. R_{f} (petroleum ether/EtOAc, 2:1) 0.74; [α]_{d}²²+2.5 (c 2.20, CHCl₃); ¹H NMR (500 MHz, CDCl₃): δ=7.52-7.33 (m, 5H, Ar), 5.83-5.68 (m, 1H, H-5), 5.51 (s, 1H, PhCH), 5.16-5.00 (m, 2H, H-6, H-6′), 4.23 (dd, J=11.4, 4.8 Hz, 1H, H-7), 3.92-3.82 (m, 1H, H-1), 3.76-3.65 (m, 2H, H-1′, H-2), 3.58 (t, J=11.4 Hz, 1H, H-7′), 2.25 (s, 1H, OH), 2.22-2.13 (m, 1H, H-4), 2.13-2.00 (m, 1H, H-3), 1.92-1.80 (m, 1H, H-4′); ¹³C NMR (126 MHz, CDCl₃): δ=134.7 (C-5), 129.3, 128.6, 126.4 (Ar—C), 117.6 (C-6), 101.4 (PhCH), 82.1 (C-2), 71.2 (C-7), 63.4 (C-1), 34.3 (C-3), 32.5 (C-5); ESI-MS: m/z: Calcd for C₁₄H₁₈O₃ [M+Na]⁺: 256.8. found: 257.1. See Akinnusi et al., Bioorg. Med. Chem. 19:2696 (2011).

### Synthesis of Compound 55

Dess-Martin periodiane (109 mg, 256 μmol) was suspended in DCM (0.5 mL) and a solution of 53 (30.0 mg, 128 μmol) in DCM (1 mL) was added. The solution was stirred for 1.5 h at rt and diluted with Et₂O. The mixture was washed with a Na₂S₂O₃ solution (1.5 g Na₂S₂O₃/5 mL satd. aq. NaHCO₃). The organic layer was dried (Na₂SO₄) and the solvents were removed in vacuo.

The crude aldehyde was dissolved in tBuOH (1 mL) and NaH₂PO₄ (24.6 mg, 205 μmol, in 165 μL H₂O). 2-Methylbut-2-ene (4 mL, 2M in THF) and NaClO₂ (37.1 mg, 410 μmol) were added successively. The resulting solution was stirred for 3 h and the volatiles evaporated under reduced pressure. The residue was dissolved in DCM (5 mL), the solution was dried (Na₂SO₄) and concentrated under reduced pressure. The resulting intermediate 6 was used without further purification in the next step.

Carboxylic acid 54 was suspended in AcOH (1 mL, 80%) and stirred for 8 h at 50° C. The solvent was removed in vacuo and the residue coevaporated with toluene and EtOH. The crude product was purified by flash chromatography (petroleum ether/EtOAc, 2:1) to afford lactone 55 (10.0 mg, 70.3 μmol, 55%) as clear oil. R_{f} (petroleum ether/EtOAc, 2:1) 0.36; [α]_{d}²²+34.2 (c 2.4, MeOH); ¹H NMR (500.1 MHz, CDCl₃): δ=5.92-5.75 (m, 1H, H-5), 5.17-5.04 (m, 2H, H-6, H-6′), 4.52 (d, J=7.3 Hz, 1H, H-2), 4.29 (dd, J=9.2, 5.8 Hz, 1H, H-7), 4.13 (dd, J=9.2, 2.9 Hz, 1H, H-7′), 2.62 (m, 1H, H-3), 2.44 (m, 1H, H-4), 1.99 (m, 1H, H-4′); ¹³C NMR (125.8 MHz, CD₃OD): δ=136.6 (C-5), 117.8 (C-6), 70.1 (C-7), 70.0 (C-2), 41.2 (C-3), 31.2 (C-4); ESI-MS: m/z: Calcd for C₇H₁₀O₃ [M+Na]⁺: 165.1. found: 164.7.

### Synthesis of Compound 56

To a solution of 55 (160 mg, 1.13 mmol) in DCM (4 mL) were added 2,6-lutidine (196 μL, 1.69 mmol) and triflic anhydride (1.26 mL, 1.12 M, 1.41 mmol) dropwise at −80° C. The resulting suspension was allowed to reach −30° C. over 3 h. The reaction was diluted with DCM (10 mL) and washed with ice-cold 1 M aq. KH₂PO₄ solution (10 mL). The aqueous layer was extracted with DCM (2×10 mL). The organic phase was dried (Na₂SO₄) and the solvent removed in vacuo. The residue was purified by flash chromatography (petroleum ether/EtOAc, 4:1) to yield triflate 56 (190 mg, 691 μmol, 61%) as a pale yellow liquid. R_{f} (petroleum ether/EtOAc, 2:1) 0.77; [α]_{D}²²+26.4 (c 0.70, CHCl₃); ¹H NMR (500.1 MHz, CDCl₃): δ=5.72 (m, 1H, H-5), 5.44 (d, J=7.4 Hz, 1H, H-2), 5.27-5.18 (m, 2H, H-6, H-6′), 4.39 (dd, J=9.6, 5.9 Hz, 1H, H-7), 4.29 (dd, J=9.6, 3.3 Hz, 1H, H-7′), 2.91 (m, 1H, H-3), 2.49 (m, 1H, H-4), 2.20 (m, 1H, H-4′); ¹³C NMR (125.8 MHz, CDCl₃): δ=168.3 (CO), 132.3 (C-5), 120.0 (C-6), 119.0 (q, J=320 Hz, CF₃), 80.0 (C-2), 68.9 (C-7), 38.9 (C-3), 30.5 (C-4).

### Synthesis of Compound 57

Diol 21 (120 mg, 0.151 mmol) and Bu₂SnO (113 mg, 0.453 mmol) were dried for 16 h at rt, then suspended in MeOH (10 mL) and refluxed for 2 h. The resulting solution was concentrated and coevaporated with toluene and the residue was dried for 16 h under reduced pressure. The tin acetal was dissolved in freshly dried (Al₂O₃ column) DME (10 mL) and added to CsF (dried for 3 h at high vacuum at 100° C., 41.3 mg, 272 μmol). Triflate 56 (62.0 mg, 0.226 mmol) was added and the suspension was stirred for 16 h at rt. A 10% solution of KF (1 M in aq. KH₂PO₄, 10 mL) was added. After stirring for 3 h at rt, DCM (12 mL) was added and the aqueous phase was extracted with DCM (2×30 ml). The combined organic layers were dried (Na₂SO₄) and concentrated under reduced pressure. Column chromatography on silica (petroleum ether/EtOAc, 1:1) afforded 57 (78.3 mg, 0.085 mmol, 56%) as a white solid. R_{f} (petroleum ether/EtOAc, 2:3) 0.53; [α]_{d}²²−30.5 (c 1.10, CHCl₃); ¹H NMR (500.1 MHz, CDCl₃): δ=7.59-7.15 (m, 20H, Ar—H), 5.75 (m, 1H, R—H5), 5.62 (s, 1H, PhCH), 5.09-5.00 (m, 2H, R—H6, R—H6′), 4.98 (d, J=3.5 Hz, 1H, Fuc-H1), 4.90 (q, J=6.7 Hz, 1H, Fuc-H5), 4.81 (d, J=11.7 Hz, 1H, PhCH₂), 4.70 (d, J=11.7 Hz, 1H, PhCH₂), 4.62-4.55 (m, 2H, 2 PhCH₂), 4.46 (dd, J=9.2, 8.0 Hz, 1H, R—H7), 4.40-4.33 (m, 3H, Gal-H1, Gal-H4, Gal-H6), 4.28 (d, J=11.3 Hz, 1H, PhCH₂), 4.15 (d, J=9.2 Hz, 1H, R—H2), 4.10 (dd, J=12.1, 1.5 Hz, 1H, Gal-H6′), 4.05 (dd, J=9.7, 3.5 Hz, 1H, Gal-H3), 3.99-3.87 (m, 4H, Fuc-H2, Fuc-H3, Gal-H2, R—H7′), 3.68-3.61 (m, 2H, MeCy-H1, PhCH₂), 3.40 (m, 1H, Gal-H5), 3.28-3.21 (m, 2H, Fuc-H4, MeCy-H2), 2.82 (m, 1H, R—H3), 2.52 (m, 1H, R—H4), 2.20 (m, 1H, R—H4′), 2.09 (m, 1H, MeCy-H6), 1.73-1.53 (m, 3H, MeCy-H5, MeCy-H3, MeCy-H4), 1.44-1.16 (m, 2H, MeCy-H6′, MeCy-H5′), 1.11-1.06 (m, 6H, Fuc-H6, MeCy-Me), 1.02 (m, 1H, MeCy-H4′); ¹³C NMR (125.8 MHz, CDCl₃): δ=175.9 (CO), 133.7 (R—C5), 129.0 (R—C6), 128.0-125.9 (Ar—C), 118.2 (R—C5), 101.3 (Fuc-C1), 99.9 (PhCH), 98.6 (Gal-C1), 82.3 (MeCy-C2), 80.4 (MeCy-C1), 79.8 (Gal-C3), 79.8 (Fuc-C3), 78.9 (Fuc-C4), 76.2 (R—C2), 75.7 (Fuc-C2), 74.9 (PhCH₂), 74.6 (PhCH₂), 72.6 (Gal-C4), 71.5 (PhCH₂), 69.8 (Gal-C6), 69.6 (R—C7), 69.5 (Gal-C2), 66.3 (Gal-C4), 66.2 (Fuc-C5), 41.7 (R—C3), 39.7 (MeCy-C3), 34.4 (R—C4), 33.9 (MeCy-C4), 31.5 (MeCy-C6), 23.5 (MeCy-C5), 19.0 (MeCy-Me), 16.7 (Fuc-C6); ESI-MS: m/z: Calcd for C₅₄H₆₄O₁₃ [M+Na]⁺: 943.4. found: 943.4.

### Synthesis of Compound 58

To a suspension of 57 (19.0 mg, 20.6 μmol) and sodium carbonate (1.1 mg, 10.3 μmol) in toluene (1.5 mL) were added vinyl acetate (1.5 mL) and chloro(1,5-cyclooctadiene)iridium (I) dimer (0.69 mg, 1.03 μmol). The mixture was refluxed at 80° C. for 48 h, then diluted with DCM (25 mL) and washed with satd. aq. NaHCO₃ (30 mL). The aqueous phase was extracted with DCM (2×15 mL). The combined organic layers were dried (Na₂SO₄) and concentrated in vacuo. The crude product was purified by flash chromatography (petroleum ether/EtOAc, 3:2+0.5% TEA) to yield 58 (8.3 mg, 8.76 μmol, 42%) as a white solid. R_{f} (petroleum ether/EtOAc, 1:1) 0.77; ¹H NMR (500.1 MHz, CDCl₃): δ=7.61-7.13 (m, 20H, Ar—H), 6.37 (dd, J=13.8, 6.3 Hz, 1H, CH₂═CH—O), 5.74 (m, 1H, R—H5), 5.64 (s, 1H, PhCH), 5.10-5.01 (m, 2H, R—H6, R—H6′), 4.97 (dd, J=11.0, 5.3 Hz, 2H, Fuc-H1, Fuc-H5), 4.81 (d, J=11.7 Hz, 1H, PhCH₂), 4.70 (d, J=11.7 Hz, 1H, PhCH₂), 4.61 (s, 2H, 2 PhCH₂), 4.50-4.45 (m, 2H, Gal-H4, R—H7), 4.42 (d, J=7.7 Hz, 1H, Gal-H1), 4.40-4.31 (m, 2H, CH₂═CH—O, Gal-H6), 4.26 (d, J=11.3 Hz, 1H, PhCH₂), 4.22-4.14 (m, 2H, R—H2, Gal-H3), 4.10 (m, 1H, Gal-H6′), 4.02-3.89 (m, 4H, Fuc-H3, CH₂═CH—O, R—H7′, Fuc-H2), 3.85 (dd, J=9.5, 7.8 Hz, 1H, Gal-H2), 3.68-3.51 (m, 2H, MeCy-H1, PhCH₂), 3.39 (m, 1H, Gal-H5), 3.28-3.19 (m, 2H, Fuc-H4, MeCy-H2), 2.78 (m, 1H, R—H3), 2.48 (m, 1H, R—H4), 2.18 (m, 1H, R—H4′), 2.02 (m, 1H, MeCy-H6), 1.69-1.51 (m, 3H, MeCy-H5, MeCy-H3, MeCy-H4), 1.40-1.12 (m, 2H, MeCy-H6′, MeCy-H5′), 1.12-1.05 (m, 6H, MeCy-Me, Fuc-H6), 1.01 (m, 1H, MeCy-H4′); ¹³C NMR (125.8 MHz, CDCl₃): δ=174.7 (CO), 153.8 (CH₂═CH—O), 139.8, 139.6, 138.8, 138.2, 129.0-125.9 (24 Ar—C), 133.9 (R—C5), 118.1 (R—C6), 100.7 (Gal-C1), 99.9 (PhCH), 98.5 (Fuc-C1), 88.8 (O—CH═CH₂), 82.0 (MeCy-C2), 81.5 (MeCy-C1), 79.8 (Fuc-C2), 79.0 (Fuc-C4), 78.8 (Gal-C2), 76.2 (Gal-C3), 75.8 (Fuc-C3), 75.2 (R—C2), 75.0 (PhCH₂), 74.6 (PhCH₂), 72.7 (Gal-C4), 71.5 (PhCH₂), 70.1 (R—C7), 69.7 (Gal-C6), 66.1 (Fuc-C5), 66.0 (Gal-C5), 41.4 (R—C3), 39.7 (MeCy-C3), 34.5 (R—C4), 33.9 (MeCy-C4), 31.2 (MeCy-C6), 23.5 (MeCy-C5), 19.0 (MeCy-Me), 16.9 (Fuc-C6); ESI-MS: m/z: Calcd for C₅₆H₆₆O₁₃ [M+Na]⁺: 969.4. found: 969.6.

### Synthesis of Compound 60

Compound 58 (8.3 mg, 8.76 mot) was dissolved in DCM (1 mL) and Grubbs 2^{nd} generation catalyst (0.74 mg, 0.88 wild) was added. The solution was stirred for 4 h at rt. The solvent was removed in vacuo and the crude product filtrated through a short pad of silica (petroleum ether/EtOAc 3:1) to give 59 (4.1 mg) as a white solid.

Compound 59 (2.1 mg, 3.95 μmol) was dissolved in dioxane/water (4:1) and Pd(OH)₂/C (1.0 mg, 10% Pd) was added. The suspension was stirred for 16 h under an atmosphere of hydrogen, filtered and concentrated. The residue was purified by flash chromatography (DCM/MeOH, 5:1). Lyophilization from water/dioxane afforded 60 (883 μg, 1.57 μmol, 35%) as a white foam. R_{f} (DCM/MeOH, 10:1.5) 0.23; [α]_{d}²²−41.2 (c 1.00, H₂O); ¹H NMR (500.1 MHz, CD₃OD): δ=5.02 (d, J=4.0 Hz, 1H, Fuc-H1), 4.95 (m, 1H, Fuc-H5), 4.57 (d, J=12.6 Hz, 1H, R—H1), 4.37 (t, J=8.0 Hz, 1H, R—H6), 4.33 (d, J=7.6 Hz, 1H, Gal-H1), 4.06 (m, 1H, Gal-H4), 3.93 (m, 1H, R—H5), 3.87 (dd, J=10.3, 3.3 Hz, 1H, Fuc-H3), 3.79-3.71 (m, 5H, R—H6′, R—H5′, Fuc-H2, Fuc-H4, Gal-H6), 3.69 (m, 1H, Gal-H6′), 3.66-3.54 (m, 2H, MeCy-H1, R—H2), 3.42-3.27 (m, 3H, Gal-H3, Gal-H5, Gal-H2), 3.21 (m, 1H, MeCy-H2), 2.34 (m, 1H, R—H4), 2.13 (m, 1H, MeCy-H6), 1.82 (m, 1H, R—H3), 1.74-1.58 (m, 4H, R—H3′, MeCy-H4, MeCy-H3, MeCy-H5), 1.47 (m, 1H, R—H4), 1.40-1.25 (m, 2H, MeCy-5′, MeCy-6′), 1.20 (d, J=6.6 Hz, 3H, Fuc-H6), 1.15 (d, J=6.4 Hz, 3H, MeCy-Me), 1.10 (m, 1H, MeCy-H4′); ¹³C NMR (125.8 MHz, CD₃OD): δ=177.4 (CO), 102.2 (Gal-C1), 100.3 (Fuc-C1), 84.1 (MeCy-C2), 81.8 (R—C1), 80.3 (MeCy-C1), 80.1 (Gal-C2), 78.5 (Gal-C3), 75.8 (Gal-C5), 75.0 (R—C5), 73.8 (Fuc-C4), 71.4 (R—C6), 71.2 (Fuc-C2), 70.4 (Gal-C4), 67.4 (Fuc-C5), 62.9 (Gal-C6), 40.8 (R—C2), 40.2 (MeCy-C3), 34.8 (MeCy-C4), 32.1 (MeCy-C6), 30.5 (R—C3), 26.6 (R—C4), 24.0 (MeCy-C5), 19.5 (MeCy-Me), 16.9 (Fuc-C6); ESI-MS: m/z: Calcd for C₂₆H₄₂O₁₃ [M+Na]⁺: 585.3. found: 585.2.

### Synthesis of Compound 61

Compound 60 (1.6 mg, 2.84 μmol) was suspended in water (500 μL) and aqueous NaOH (0.1 M, 57 μL, 5.69 μmol) was added. The resulting solution was stirred for 16 h at rt and concentrated. The residue was purified via RP chromatography (MeOH/H₂O). Lyophilization from water/dioxane afforded 61 (0.50 mg, 0.83 μmol, 29%) as a white solid. R_{f} (DCM/MeOH/H₂O, 10:5:0.4) 0.16; [α]_{D}²²−80.7 (c 0.26, H₂O); ¹H NMR (500.1 MHz, D₂O): δ 5.14 (d, J=4.1 Hz, 1H, Fuc-H1), 4.94-4.88 (m, 1H, Fuc-H5S), 4.54 (d, J=7.8 Hz, 1H, Gal-H1), 4.16 (dd, J=9.8, 3.4 Hz, 1H, Gal-H3), 4.00 (d, J=10.9 Hz, 114, R—H1), 3.93 (dd, J=10.6, 3.3 Hz, 1H, Fuc-H3), 3.91-3.88 (m, 1H, Gal-H4), 3.86-3.81 (m, 2H, Fuc-H4, Fuc-H2), 3.79-3.66 (m, 5H, R—H5, R—H5′, MeCy-H1, Gal-H6, Gal-H6′), 3.59 (dd, J=11.2, 3.5 Hz, 1H, R—H6), 3.56-3.52 (m, 1H, Gal-H5), 3.42-3.36 (m, 2H, R—H6′, Gal-H2), 3.26 (t, J=9.4 Hz, 1H, MeCy-H2), 2.34-2.27 (m, 1H, R—H2), 2.22-2.13 (m, 2H, R—H4), 1.90-1.80 (m, 1H, R—H3), 1.79-1.59 (m, 4H, R—H3′, MeCy-H4, MeCy-H3, MeCy-H5), 1.56-1.49 (m, 1H, R—H4′), 1.40-1.25 (m, 2H, MeCy-H5′, MeCy-H6′), 1.23 (d, J=7.4 Hz, 3H, Fuc-H6), 1.12 (d, J=6.4 Hz, 31-1, MeCy-Me), 1.16-1.06 (m, 1H, MeCy-H4′); ¹³C NMR (125.8 MHz, D₂O): δ 99.4 (Gal-C1), 98.1 (Fuc-C1), 83.5 (MeCy-C2), 82.0 (R—C1), 78.7 (MeCy-C1), 77.5 (Gal-C2), 74.0 (Gal-C5), 73.8 (Gal-C3), 71.6 (Fuc-C2), 69.1 (Fuc-C3), 68.5 (Gal-C4), 68.0 (Fuc-C4), 66.3 (Fuc-C5), 63.7 (R—C6), 61.6 (Gal-C6), 39.5 (R—C2), 38.5 (MeCy-C3), 32.6 (MeCy-C4), 30.3 (MeCy-C6), 28.6 (R—C3), 23.5 (R—C4), 23.0 (MeCy-C5), 18.2 (MeCy-Me), 15.7 (Fuc-C6); ESI-MS: m/z: Calcd for C₂₆H₄₃NaO₁₄ [M−Na]⁻: 579.3. found: 579.2.

### Example 2

### E-Selectin Activity Binding Assay

The inhibition assay to screen and characterize glycomimetic antagonists of E-selectin is a competitive binding assay, from which IC₅₀ values may be determined. E-selectin/Ig chimera was immobilized in 96 well microtiter plates by incubation at 37° C. for 2 hours. To reduce nonspecific binding, bovine serum albumin was added to each well and incubated at room temperature for 2 hours. The plate was washed and serial dilutions of the test compounds were added to the wells in the presence of conjugates of biotinylated, sLe^{a} polyacrylamide with streptavidin/horseradish peroxidase and incubated for 2 hours at room temperature.

To determine the amount of sLe^{a} bound to immobilized E-selectin after washing, the peroxidase substrate, 3,3′,5,5′ tetramethylbenzidine (TMB) was added. After 3 minutes, the enzyme reaction was stopped by the addition of H₃PO₄, and the absorbance of light at a wavelength of 450 nm was determined. The concentration of test compound required to inhibit binding by 50% was determined and reported as the IC₅₀ value for each glycomimetic E-selectin antagonist as shown in the table below. IC₅₀ values for exemplary compounds disclosed herein are provided in the following table.

### E-Selectin Antagonist Activity of Glycomimetic Compounds

| Compound | IC50 (μM) | rIC50 |
|---|---|---|
| 18a | 15.2 | 3.04 |
| 25 | 15.8 | 2.35 |
| 39 | 7.3 | 1.30 |
| 47 | 3.8 | 1.52 |

The various embodiments described above can be combined to provide further embodiments. All U.S. patents, U.S. patent application publications, U.S. patent applications, non-U.S. patents, non-U.S. patent applications, and non-patent publications referred to in this specification and/or listed in the Application Data Sheet are incorporated herein by reference, in their entirety. Aspects of the embodiments can be modified, if necessary, to employ concepts of the various patents, applications, and publications to provide yet further embodiments.

What is claimed is:

1. At least one compound chosen from compounds of Formula (I): 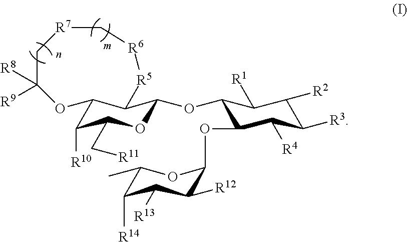
wherein
R¹ is chosen from H, C₁₋₈ alkyl, C₂₋₈ alkenyl, C₂₋₈ alkynyl, C₂₋₈ haloalkyl, C₂₋₈ haloalkenyl, and C₂₋₈ haloalkynyl groups;
R² is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, -M, -L-M, —C(═O)OY¹, and —C(═O)NY¹Y² groups, wherein Y¹ and Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y¹ and Y² may join together to form a ring;
R³ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, -M, -L-M, —C(═O)OY³, and —C(═O)NY³Y⁴ groups, wherein Y³ and Y⁴, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y³ and Y⁴ may join together to form a ring;
R⁴ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R⁵ is chosen from 0, S, and NR¹⁵;
R⁶ is chosen from a bond, C(═O), and CR¹⁶R¹⁷;
R⁷ is chosen from C₂₋₈ alkylene, C₂₋₈ alkenyl, C₂₋₈ alkynyl, C₂₋₁₂ heterocyclyl, C₆₋₁₈ aryl, C₂₋₁₃ heteroaryl, and CR¹⁸R¹⁹ groups;
R⁸ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₁₋₈ alkoxy, C₆₋₁₈ aryl, and C₂₋₁₃ heteroaryl groups, or R⁸ joins together with R⁹ to form a ring;
R⁹ is chosen from —Z, —CH₂OH, —CH₂OY⁵, OH, —OY⁵, —CN, —C(═O)Y⁵, —C(═O)OH, —C(═O)OY⁵, —C(═O)NY⁵Y⁶, —S(═O)₂Y⁵, —S(═O)₂OY⁵, and —S(═O)₂NY⁵Y⁶ groups, wherein Y⁵ and Y⁶, which may be identical or different, are independently chosen from C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y⁵ and Y⁶ may join together to form a ring, or R⁹ joins together with R⁸ or R¹⁸ to form a ring;
R¹⁰ is chosen from H, —OH, F, Cl, Br, —CF₂H, and —NY⁷Y⁸, wherein Y⁷ and Y⁸, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y⁷ and Y⁸ may join together to form a ring;
R¹¹ is chosen from H, —OH, F, Cl, Br, —CF₂H, and —NY⁹Y¹⁰, wherein Y⁹ and Y¹⁰, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y⁹ and Y¹⁰ may join together to form a ring;
R¹² is chosen from —OH, F, Cl, Br, —CF₂H, and —NY¹¹Y¹², wherein Y¹¹ and Y¹², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y¹¹ and Y¹² may join together to form a ring;
R¹³ is chosen from —OH, F, Cl, Br, —CF₂H, and —NY¹³Y¹⁴, wherein Y¹³ and Y¹⁴, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y¹³ and Y¹⁴ may join together to form a ring;
R¹⁴ is chosen from —OH, F, Cl, Br, —CF₂H, and —NY⁷Y⁸, wherein Y¹⁵ and Y¹⁶, which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, and C₂₋₁₂ haloalkynyl groups, wherein Y¹⁵ and Y¹⁶ may join together to form a ring;
R¹⁵ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
R¹⁶ is chosen from H, C₁₋₈ alkyl, C₂₋₈ alkenyl, C₂₋₈ alkynyl, C₁₋₄ haloalkyl, C₂₋₈ haloalkenyl, and C₂₋₄ haloalkynyl groups, or R¹⁶ joins together with R¹⁷ to form a ring;
R¹⁷ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, or R¹⁷ joins together with R¹⁶ to form a ring;
R¹⁸ is chosen from H, C₁₋₈ alkyl, C₂₋₈ alkenyl, C₂₋₈ alkynyl, C₁₋₄ haloalkyl, C₂₋₄ haloalkenyl, C₂₋₈ haloalkynyl, and C₁₋₄ alkoxy groups, or R¹⁸ joins together with R⁹ or R¹⁹ to form a ring;
R¹⁹ is chosen from H, C₁₋₈ alkyl, C₂₋₈ alkenyl, C₂₋₈ alkynyl, C₁₋₄ haloalkyl, C₂₋₈ haloalkenyl, C₂₋₈ haloalkynyl, and C₁₋₈ alkoxy groups, or R¹⁹ joins together with R¹⁸ to form a ring;
L is chosen from linker groups;
M is chosen from non-glycomimetic moieties;
Z is chosen from acid bioisosteric moieties;
m is chosen from integers ranging from 0 to 5; and
n is chosen from integers ranging from 0 to 5.

2. The at least one compound according to claim 1, wherein R¹², R¹³, and/or R¹⁴ is OH.

3. The at least one compound according to claim 1, wherein R¹², R¹³, and R¹⁴ are each OH.

4. The at least one compound according to any one of claims 1 to 3, wherein R¹ is chosen from H, C₁₋₄ alkyl, and C₁₋₄ haloalkyl groups.

5. The at least one compound according to any one of claims 1 to 4, wherein R² is chosen from H, —C(═O)OY¹, and —C(═O)NY¹Y².

6. The at least one compound according to any one of claims 1 to 5, wherein R³ is chosen from H, —C(═O)OY¹, and —C(═O)NY¹Y².

7. The at least one compound according to any one of claims 1 to 6, wherein R⁴ is chosen from H, C₁₋₄ alkyl, and C₁₋₄ haloalkyl groups.

8. The at least one compound according to any one of claims 1 to 7, wherein R′ and/or R⁴ is chosen from H, methyl, and ethyl.

9. The at least one compound according to any one of claims 1 to 8, wherein R⁴ is chosen from methyl and ethyl.

10. The at least one compound according to any one of claims 1 to 9, wherein R¹, R², and/or R³ is H.

11. The at least one compound according to any one of claims 1 to 9, wherein R¹, R², and R³ are each H.

12. The at least one compound according to any one of claims 1 to 11, wherein m and n are chosen such that the sum of m and n is an integer ranging from 0 to 5.

13. The at least one compound according to any one of claims 1 to 12, wherein R¹⁰ is chosen from H, —OH, F, and —CF₂H.

14. The at least one compound according to any one of claims 1 to 12, wherein R¹¹ is chosen from H, —OH, and —CF₂H.

15. The at least one compound according to any one of claims 1 to 14, wherein R⁶ is chosen from CR¹⁶R¹⁷ groups.

16. The at least one compound according to claim 15, wherein R¹⁶ is chosen from H and C₁₋₈ alkyl groups, or R¹⁶ joins together with R¹⁷ to form a ring.

17. The at least one compound according to claim 15 or 16, wherein R¹⁷ is chosen from H and C₁₋₈ alkyl groups, or R¹⁶ joins together with R¹⁷ to form a ring.

18. The at least one compound according to claim 15, wherein R¹⁶ joins together with R¹⁷ to form a ring, and the at least one compound is chosen from compounds of Formula (Ia):

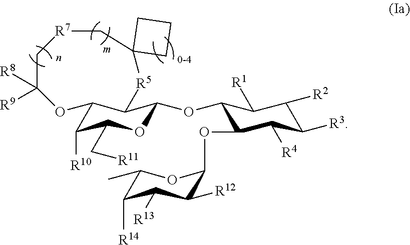

19. The at least one compound according to claim 1, wherein the at least one compound is chosen from compounds of the following Formulas: 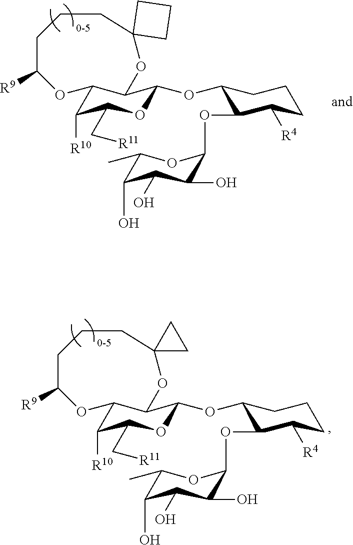
wherein
R⁴ is chosen from H, C₁₋₄ alkyl, and C₁₋₄ haloalkyl groups;
R⁹ is chosen from —Z, —C(═O)OH, —C(═O)OY⁵; and —C(═O)NY⁵Y⁶;
R¹⁰ is chosen from H, —OH, F, and —CF₂H; and
R¹¹ is chosen from H, —OH, and —CF₂H.

20. The at least one compound according to any one of claims 1 to 18, wherein R⁷ is chosen from CR¹⁸R¹⁹ groups.

21. The at least one compound according to claim 20, wherein R¹⁸ is chosen from H and C₁₋₈ alkyl groups, or R¹⁸ joins together with R¹⁹ to form a ring.

22. The at least one compound according to any one of claims 20 and 21, wherein R¹⁹ is chosen from H and C₁₋₈ alkyl groups, or R¹⁹ joins together with R¹⁸ to form a ring.

23. The at least one compound according to any one of claims 20 and 21, wherein R¹⁸ joins together with R¹⁹ to form a ring, and the at least one compound is chosen from compounds of Formula (Ib):

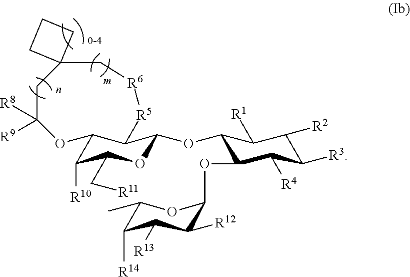

24. The at least one compound according to claim 1, wherein the at least one compound is chosen from compounds of the following Formulas: 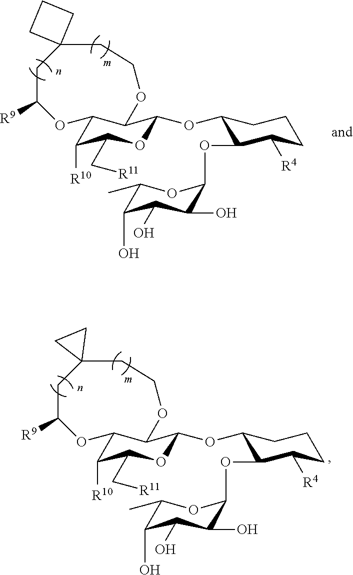
wherein
R⁴ is chosen from H, C₁₋₄ alkyl, and C₁₋₄ haloalkyl groups;
R⁹ is chosen from —Z, —C(═O)OH, —C(═O)OY⁵, and —C(═O)NY⁵Y⁶;
R¹⁰ is chosen from H, —OH, F, and —CF₂H;
R¹¹ is chosen from H, —OH, and —CF₂H; and
m and n are chosen such that the sum of m and n is an integer ranging from 0 to 6.

25. The at least one compound according to any one of claims 1 to 15, wherein the at least one compound is chosen from compounds of Formula (Ic):

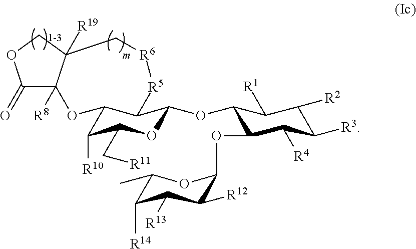

26. The at least one compound according to claim 1, wherein the at least one compound is chosen from compounds of the following Formula: 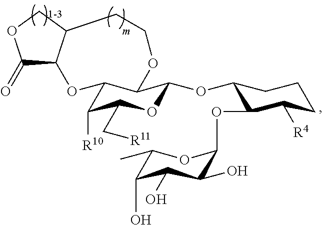
wherein
R⁴ is chosen from H, C₁₋₄ alkyl, and C₁₋₄ haloalkyl groups;
R¹⁰ is chosen from H, —OH, F, and —CF₂H; and
R¹¹ is chosen from H, —OH, and —CF₂H.

27. The at least one compound according to any one of claims 1 to 15, wherein the at least one compound is chosen from compounds of Formulas (Id), (Ie), and (If): 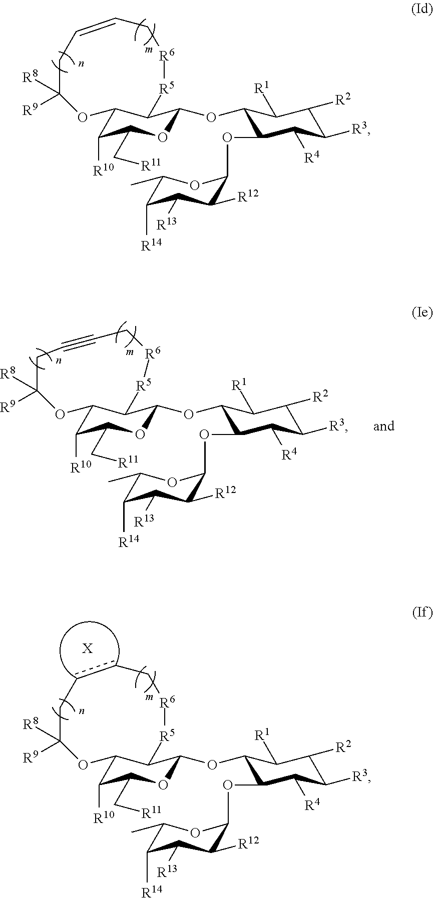
wherein X represents a carbocyclic, heterocyclic, aromatic, or heteroaromatic ring.

28. The at least one compound according to claim 1, wherein the at least one compound is chosen from compounds of the following Formula: 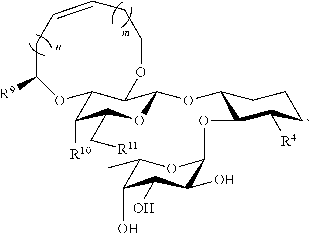
wherein
R⁴ is chosen from H, C₁₋₄ alkyl, and C₁₋₄ haloalkyl groups;
R⁹ is chosen from —Z, —C(═O)OH, —C(═O)OY⁵, and —C(═O)NY⁵Y⁶;
R¹⁰ is chosen from H, —OH, F, and —CF₂H;
R¹¹ is chosen from H, —OH, and —CF₂H; and
m and n are chosen such that the sum of m and n is an integer ranging from 0 to 5.

29. The at least one compound according to claim 1, wherein the at least one compound is chosen from compounds of the following Formula: 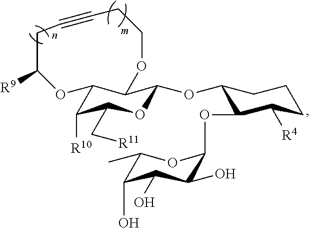
wherein
R⁴ is chosen from H, C₁₋₄ alkyl, and C₁₋₄ haloalkyl groups;
R⁹ is chosen from —Z, —C(═O)OH, —C(═O)OY⁵, and —C(═O)NY⁵Y⁶;
R¹⁰ is chosen from H, —OH, F, and —CF₂H;
R¹¹ is chosen from H, —OH, and —CF₂H; and
m and n are chosen such that the sum of m and n is an integer ranging from 0 to 5.

30. The at least one compound according to claim 1, wherein the at least one compound is chosen from compounds of the following Formulas: 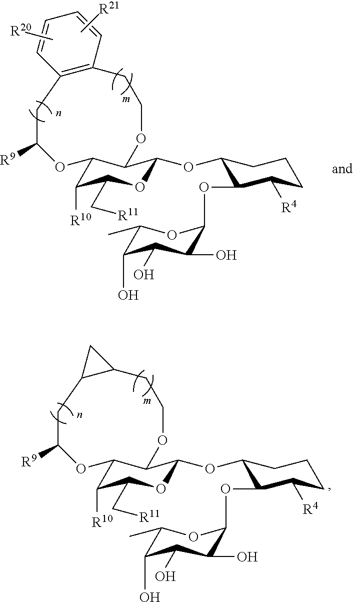
wherein
R⁴ is chosen from H, C₁₋₄ alkyl, and C₁₋₄ haloalkyl;
R⁹ is chosen from —Z, —C(═O)OH, —C(═O)OY⁵, and —C(═O)NY⁵Y⁶;
R¹⁰ is chosen from H, —OH, F, and —CF₂H;
R¹¹ is chosen from H, —OH, and —CF₂H;
R²⁰ is chosen from H, halo, C₁₋₆ alkyl, C₁₋₆ haloalkyl, —OH, —O—C₁₋₆ alkyl, C₂₋₆ heterocyclyl, C₆₋₁₀ aryl, C₂₋₈ heteroaryl, and —C(═O)OY¹⁷ groups, wherein Y¹⁷ is chosen from H, C₁₋₆ alkyl, C₂₋₁₂ heterocyclyl, C₆₋₁₀ aryl, and C₂₋₈ heteroaryl groups;
R²¹ is chosen from H, halo, C₁₋₆ alkyl, C₁₋₆ haloalkyl, —OH, —O—C₁₋₆ alkyl, C₂₋₆ heterocyclyl, C₆₋₁₀ aryl, C₂₋₈ heteroaryl, and —C(═O)OY¹⁸ groups, wherein Y¹⁸ is chosen from H, C₁₋₆ alkyl, C₂₋₁₂ heterocyclyl, C₆₋₁₀ aryl, and C₂₋₈ heteroaryl groups; and
m and n are chosen such that the sum of m and n is an integer ranging from 0 to 5.

31. The at least one compound according to any one of claims 1 to 18, 20 to 23, 25, and 27, wherein R⁸ is chosen from H, C₁₋₄ alkyl, C₆₋₁₈ aryl, and C₂₋₁₃ heteroaryl groups.

32. The at least one compound according to any one of claims 1 to 15, wherein the at least one compound is chosen from compounds of Formulas (Ig) and (Ih):

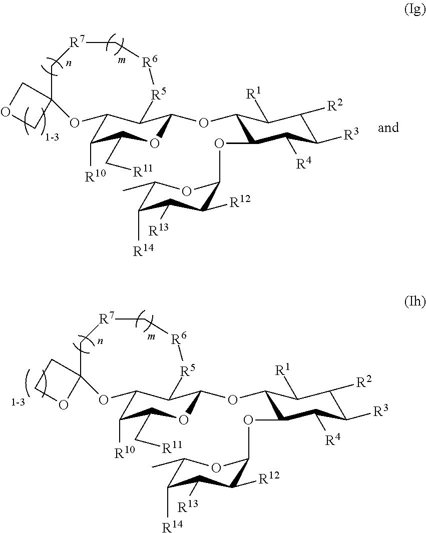

33. The at least one compound according to claim 1, wherein the at least one compound is chosen from compounds of the following Formulas:

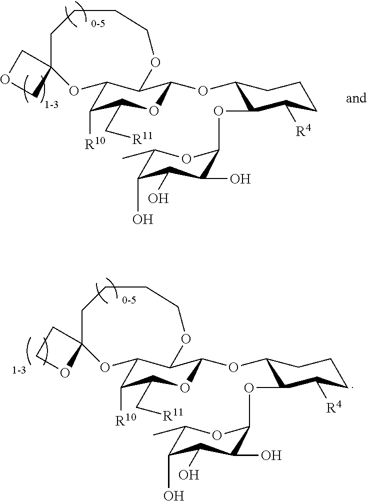

34. The at least one compound according to any one of claims 1 to 18, 20 to 23, 25, 27, 31, and 32, wherein R⁵ is O.

35. The at least one compound according to any one of claims 19, 24, 26, and 28 to 34, wherein R⁴ is methyl.

36. The at least one compound according to any one of claims 19, 24, 26, and 28 to 34, wherein R⁴ is ethyl.

37. The at least one compound according to any one of claims 19, 24, 26, and 28 to 36, wherein R¹⁰ is —OH.

38. The at least one compound according to any one of claims 19, 24, 26, and 28 to 37, wherein R¹¹ is —OH.

39. The at least one compound according to claim 1, wherein the at least one compound is chosen from compounds of the following Formulas:

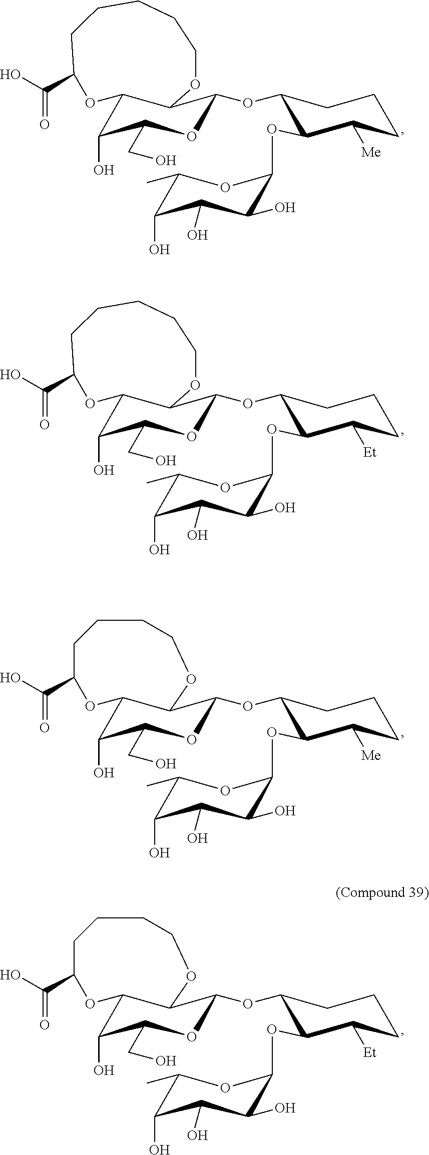

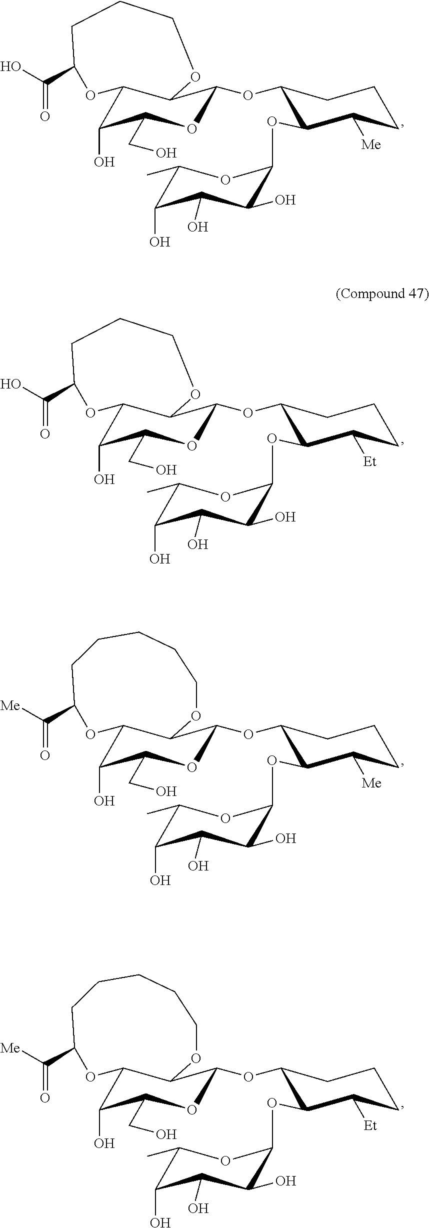

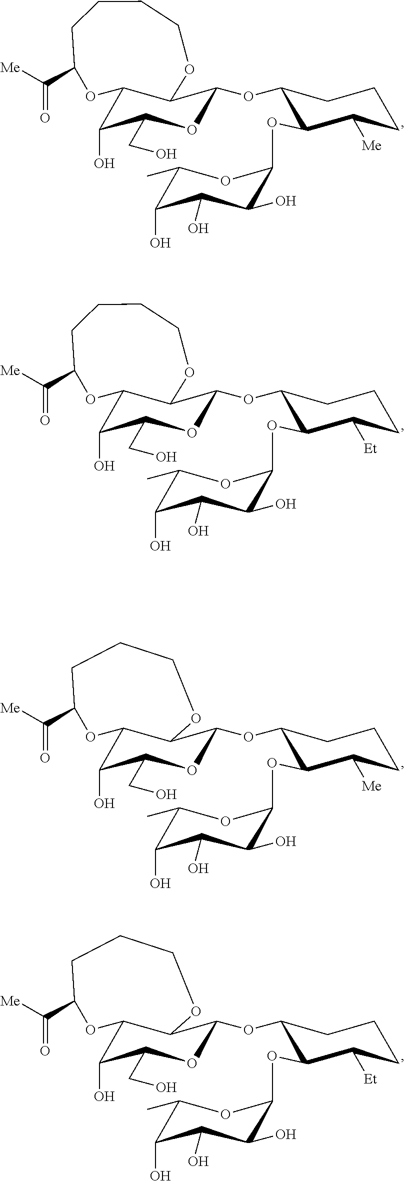

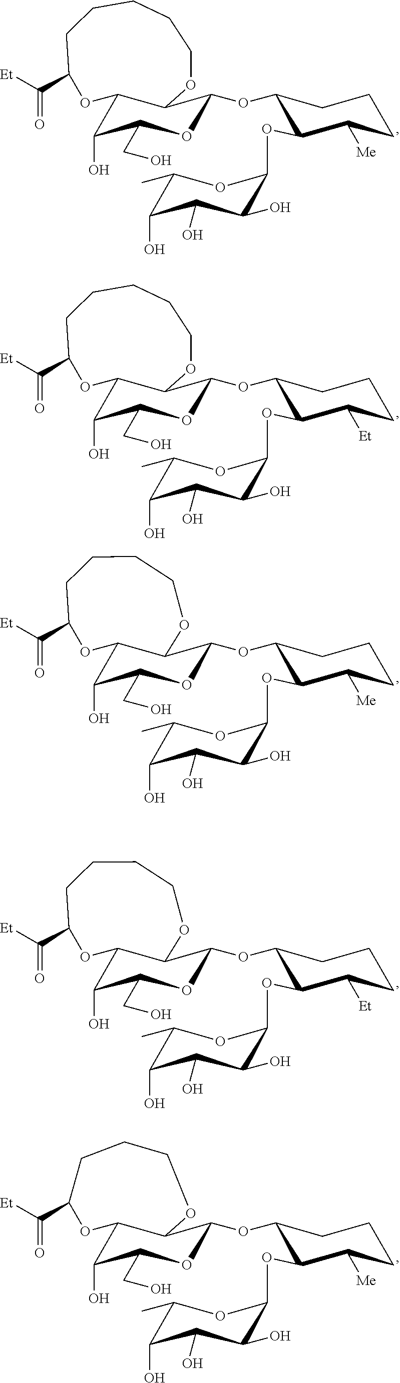

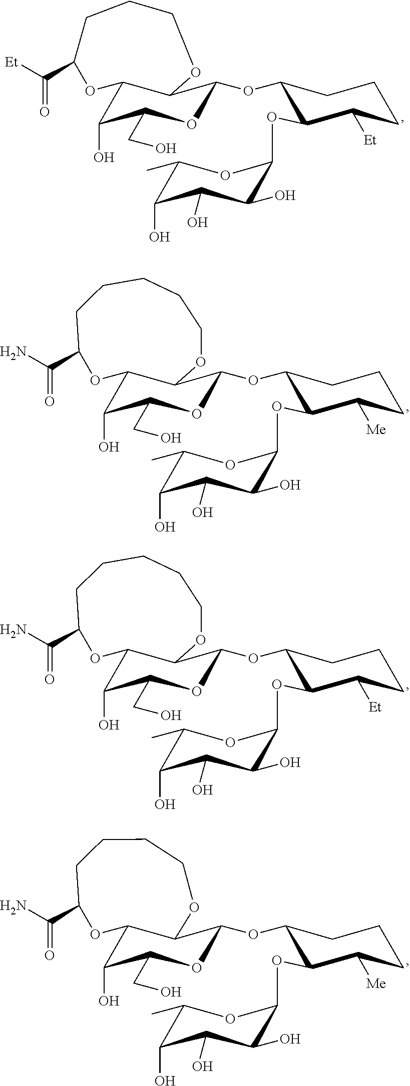

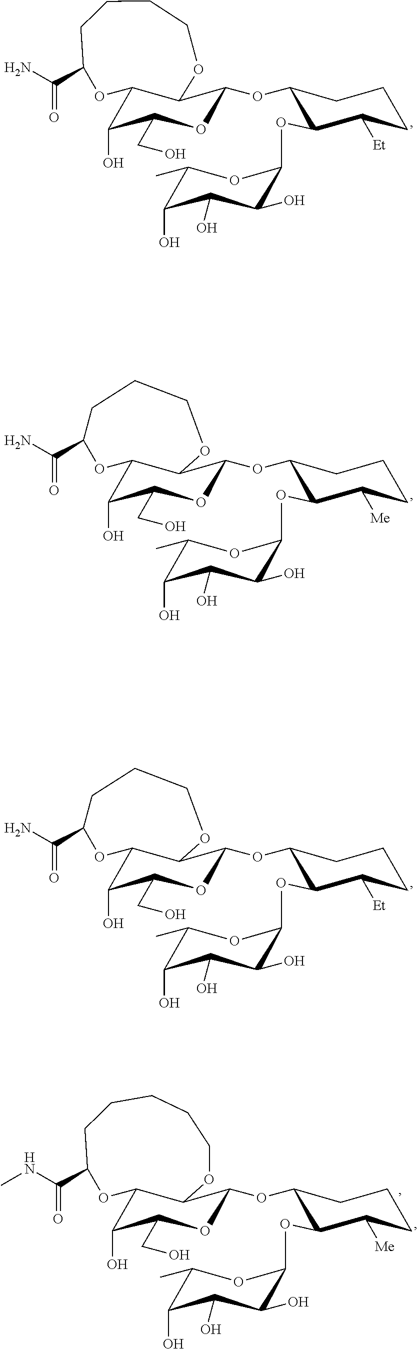

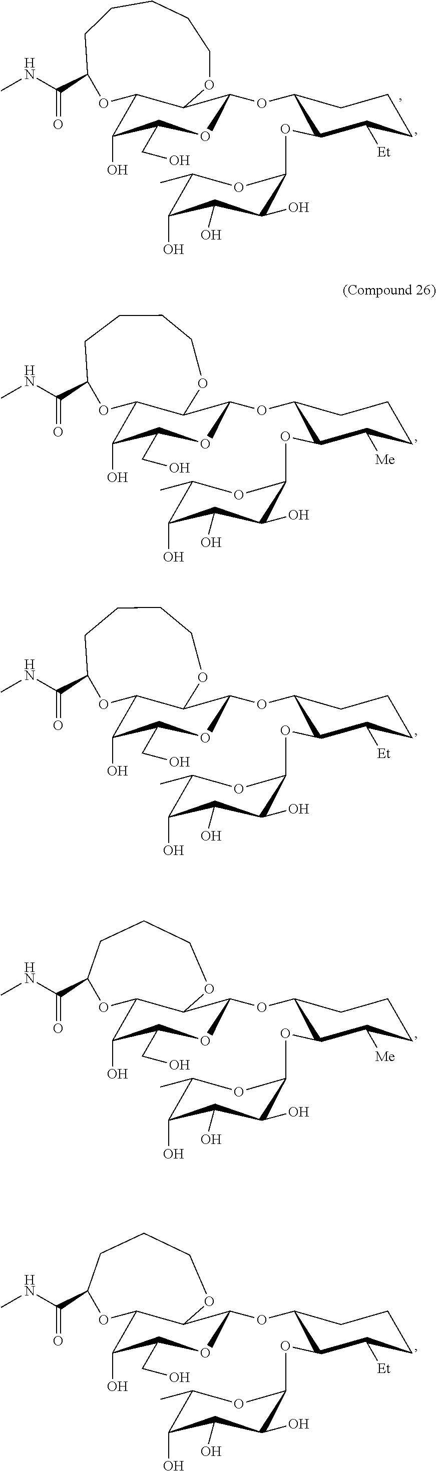

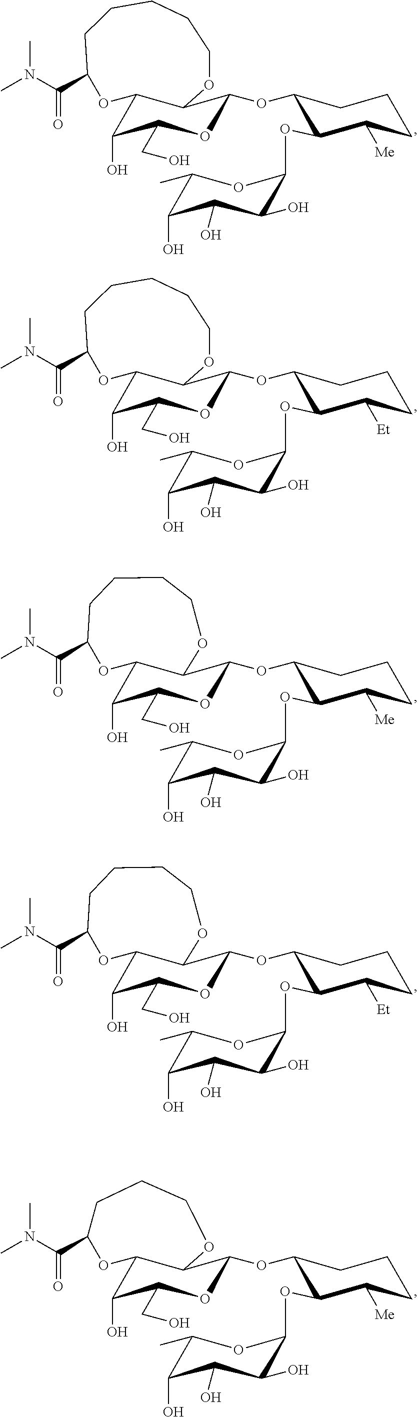

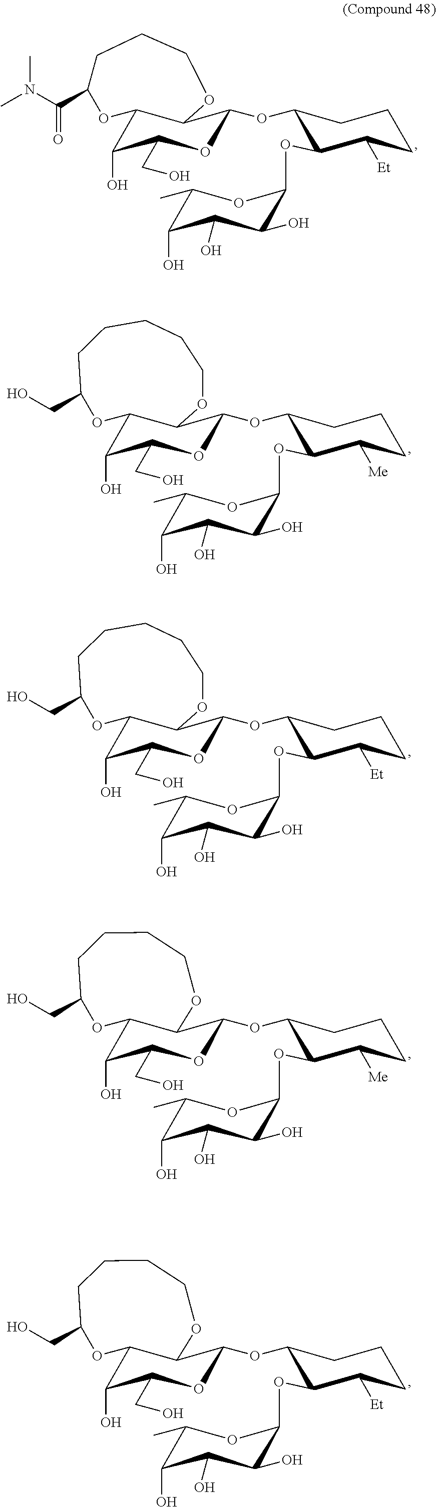

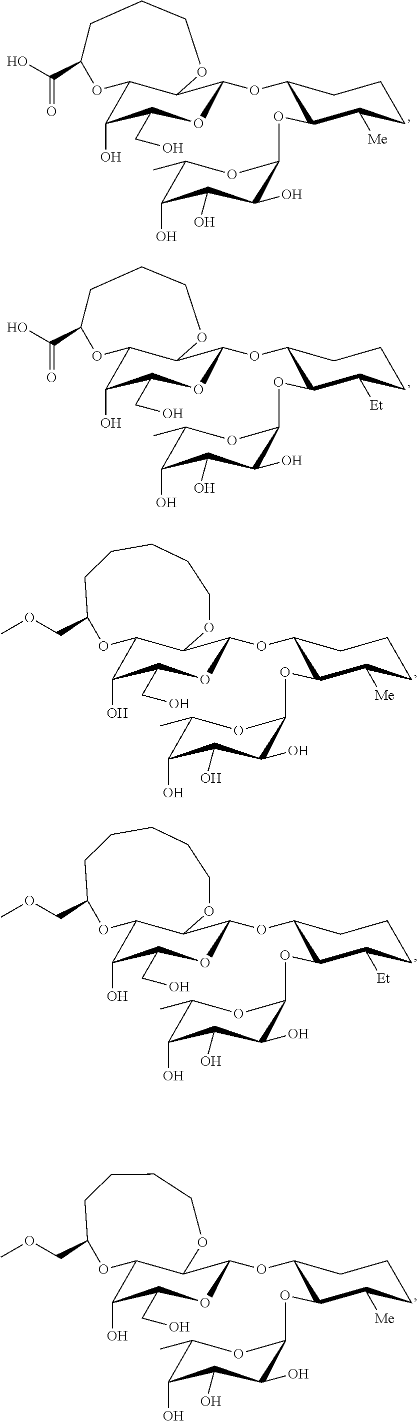

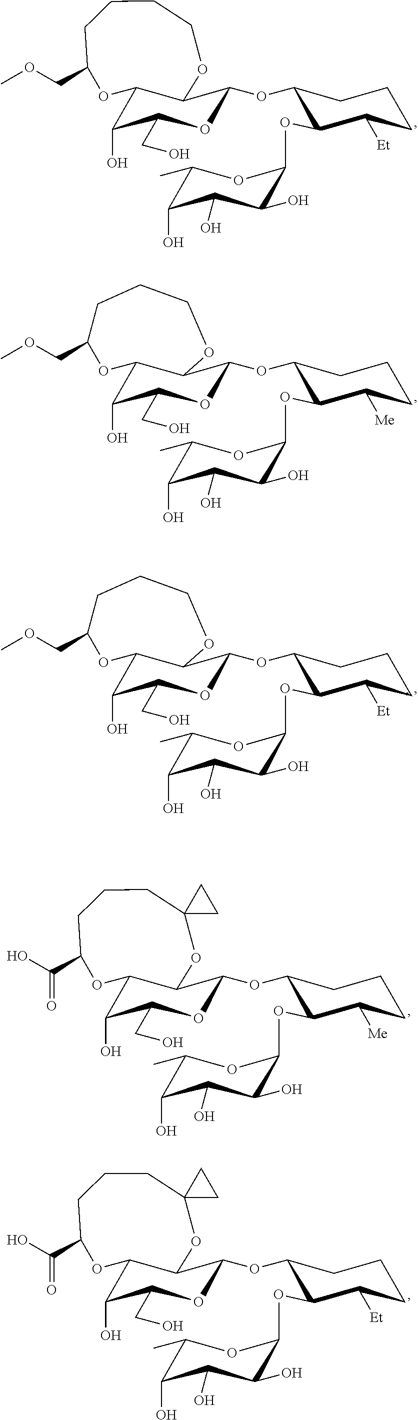

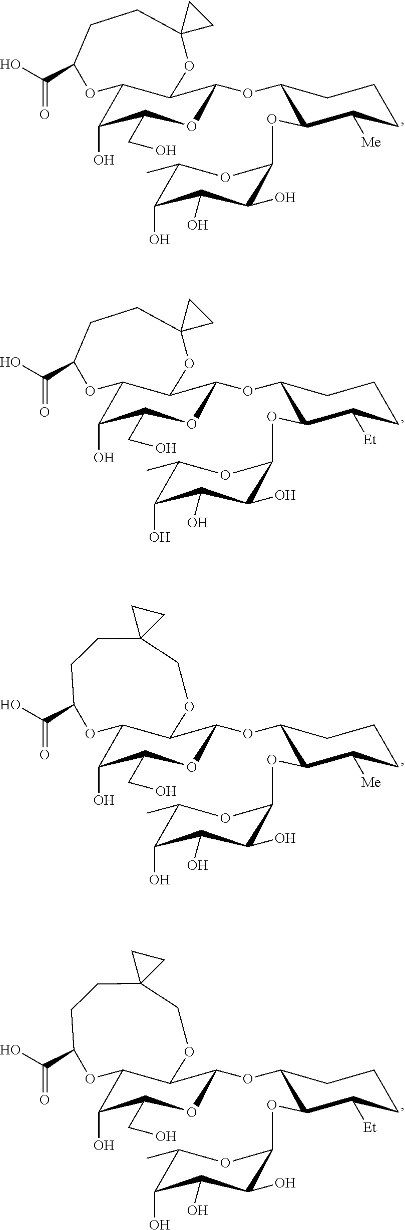

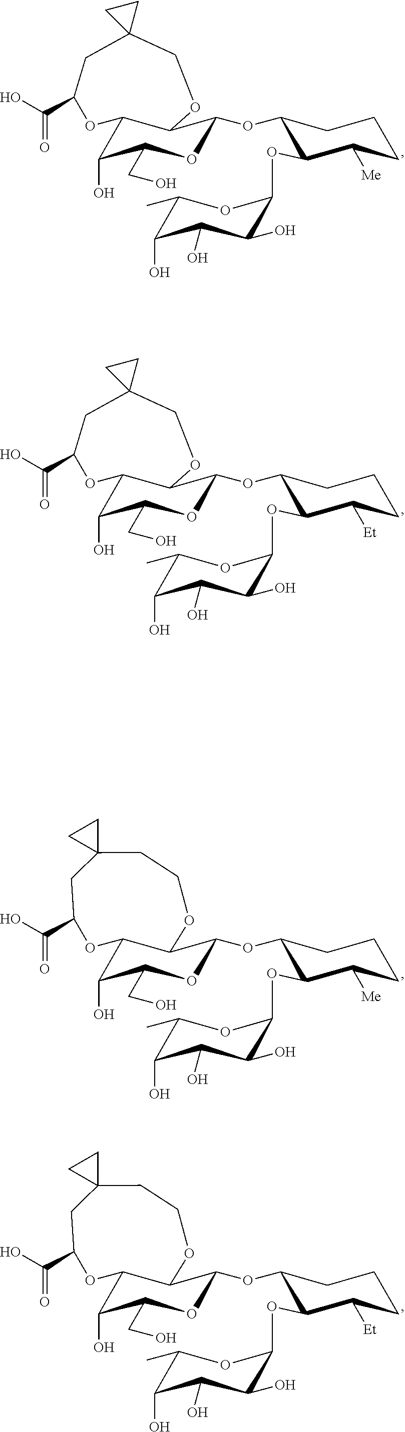

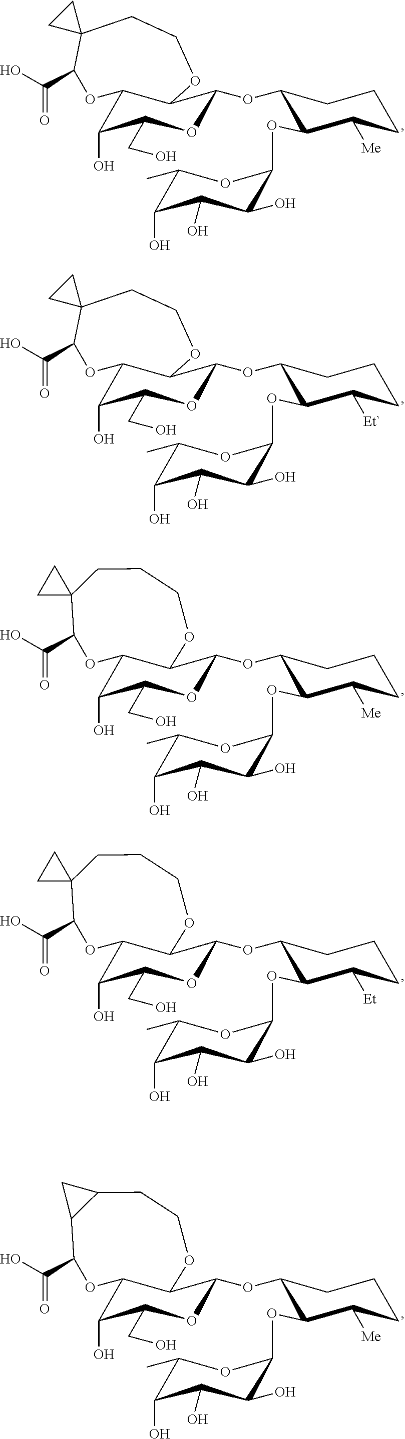

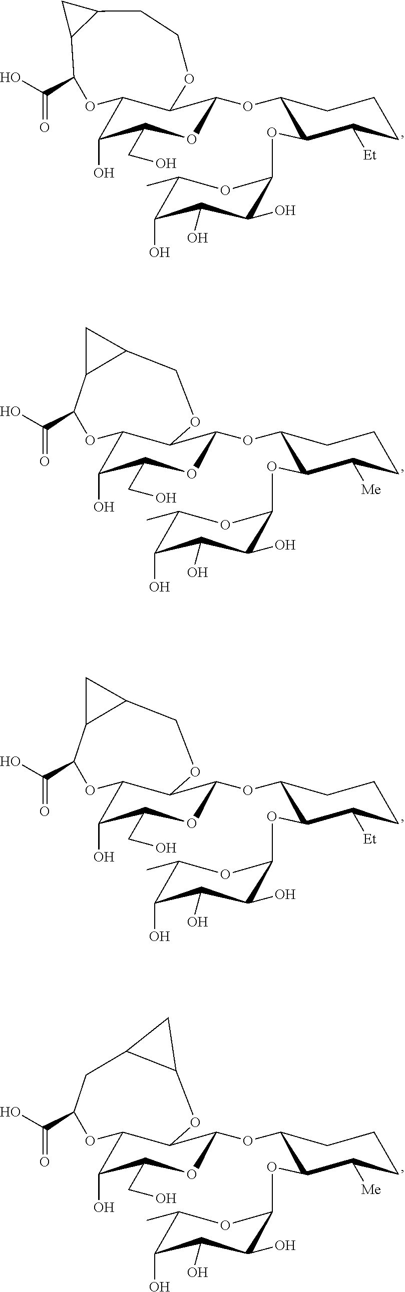

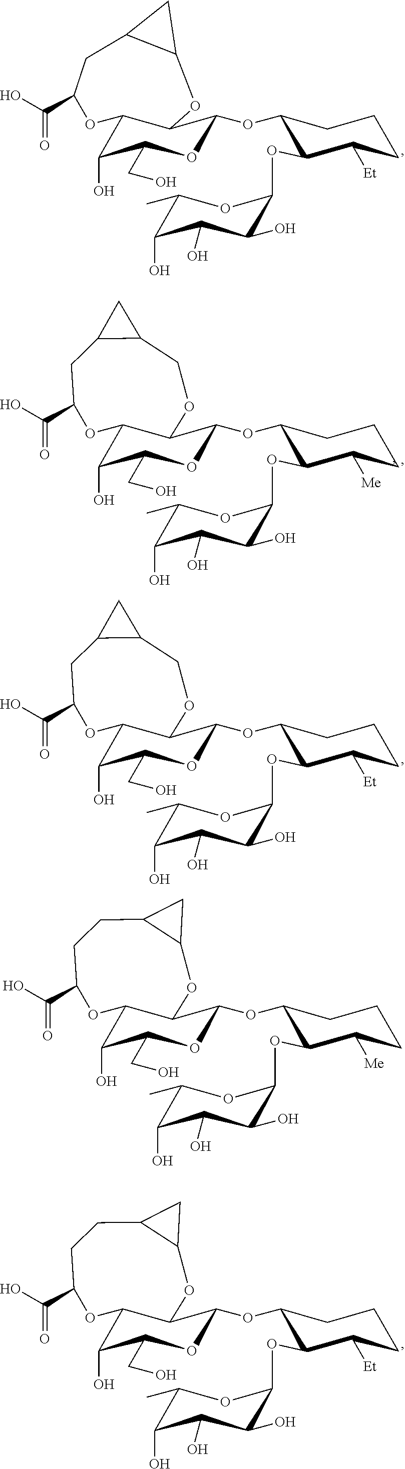

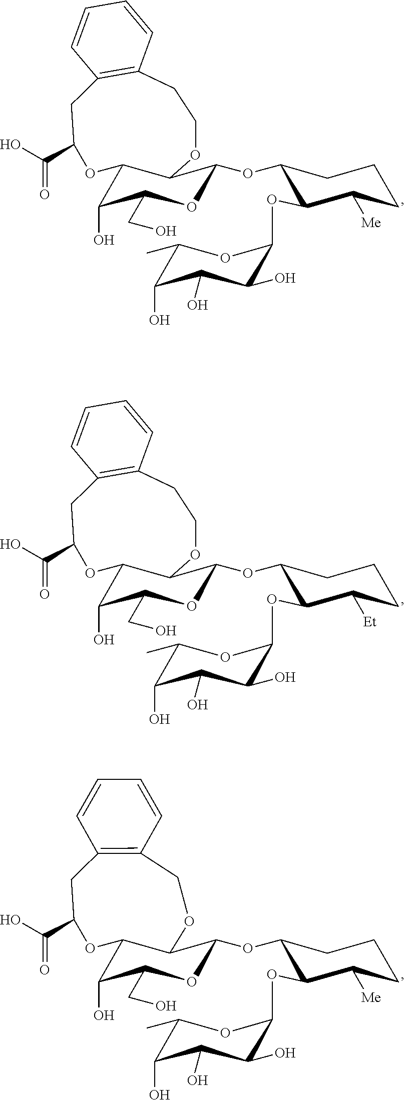

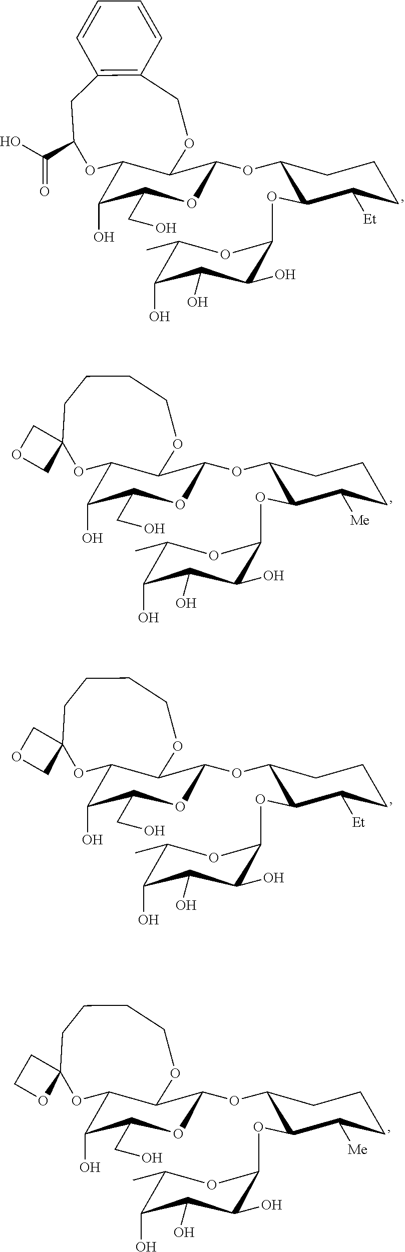

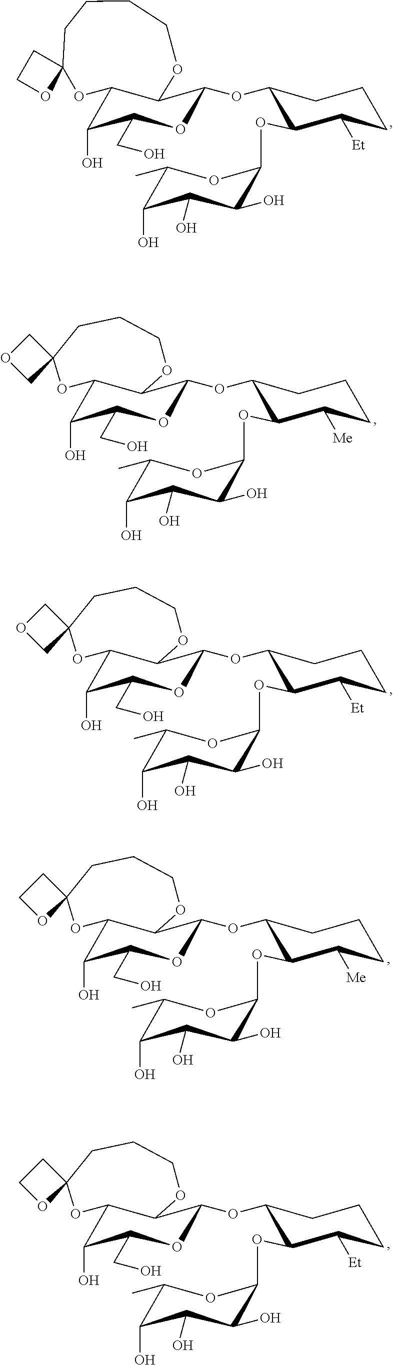

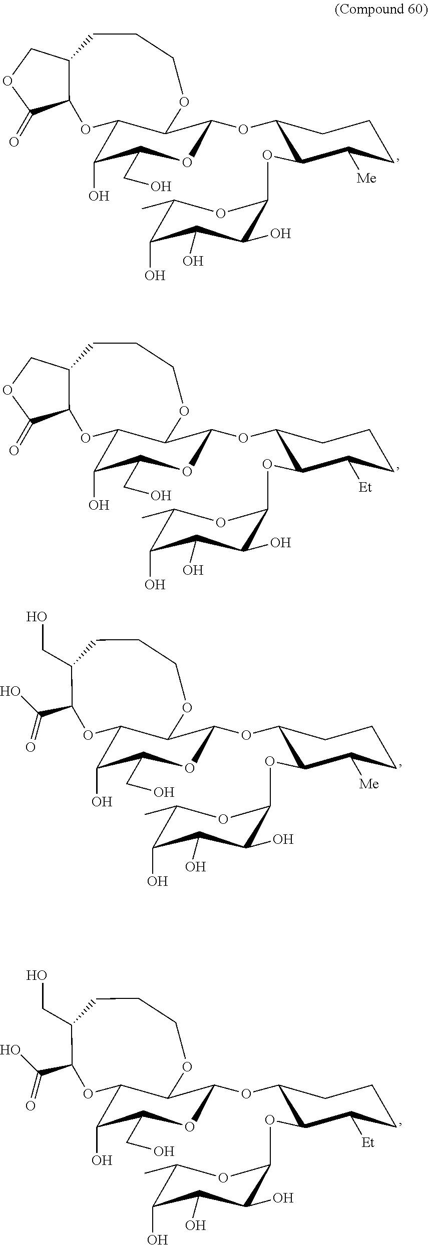

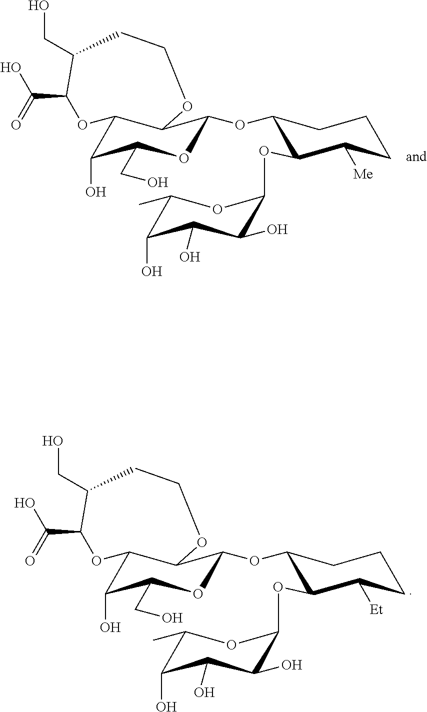

40. The at least one compound according to claim 1, wherein the at least one compound is chosen from compounds of the following Formulas:

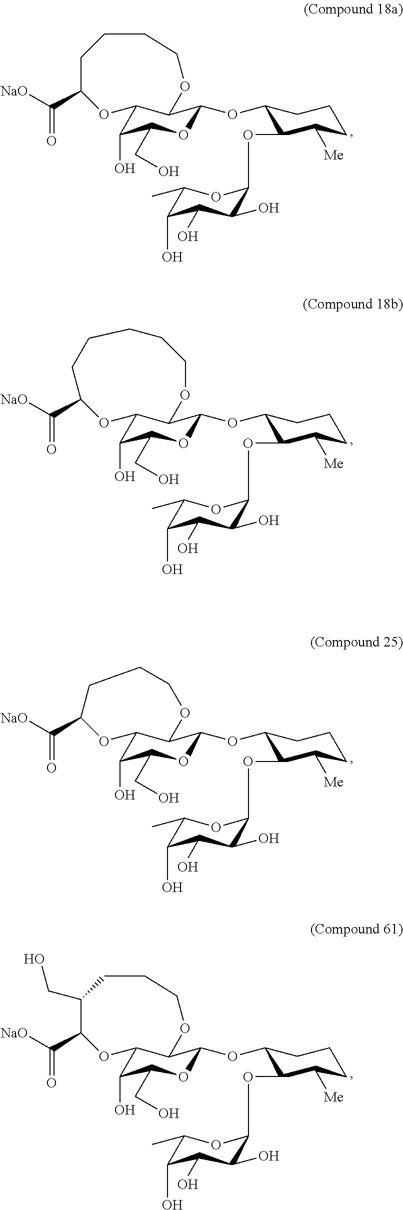

41. A composition comprising at least one compound of any of claims 1 to 40 and at least one pharmaceutically acceptable ingredient.

42. A method for the treatment and/or prevention of a disease or condition where inhibition of E-selectin mediated functions is useful comprising administering to a subject in need thereof an effective amount of at least one compound of any one of claims 1 to 40 and optionally at least one pharmaceutically acceptable ingredient.

43. A method for the treatment and/or prevention of an inflammatory disease or disorder comprising administering to a subject in need thereof an effective amount of at least one compound of any one of claims 1 to 40 and optionally at least one pharmaceutically acceptable ingredient.

44. A method for the treatment and/or prevention of metastasis of cancer cells comprising administering to a subject in need thereof an effective amount of at least one compound of any one of claims 1 to 40 and optionally at least one pharmaceutically acceptable ingredient.

45. A method for inhibiting infiltration of cancer cells into bone marrow comprising administering to a subject in need thereof an effective amount of at least one compound of any one of claims 1 to 40 and optionally at least one pharmaceutically acceptable ingredient.

46. A method for inhibiting adhesion of a tumor cell that expresses a ligand of E-selectin to an endothelial cell expressing E-selectin, the method comprising contacting the endothelial cell with an effective amount of at least one compound of any one of claims 1 to 40 and optionally at least one pharmaceutically acceptable ingredient.

47. The method according to claim 46, wherein the endothelial cell is present in bone marrow.

48. A method for the treatment and/or prevention of thrombosis comprising administering to a subject in need thereof an effective amount of at least one compound of any one of claims 1 to 40 and optionally at least one pharmaceutically acceptable ingredient.

49. A method for the treatment and/or prevention of cancer comprising administering to a subject in need thereof (a) an effective amount of at least one compound according to any one of claims 1 to 40 and optionally at least one pharmaceutically acceptable ingredient and (b) at least one of therapy chosen from (i) chemotherapy and (ii) radiotherapy.

50. A method for enhancing hematopoietic stem cell survival comprising administering to a subject in need thereof an effective amount of at least one compound of any one of claims 1 to 40 and optionally at least one pharmaceutically acceptable ingredient.

51. The method according to claim 50, wherein the subject has cancer and has received or will receive chemotherapy and/or radiotherapy.

52. A method for treating and/or preventing mucositis comprising administering to a subject in need thereof an effective amount of at least one compound of any one of claims 1 to 40 and optionally at least one pharmaceutically acceptable ingredient.

53. The method according to claim 52, wherein the mucositis is oral mucositis, esophageal mucositis, and/or gastrointestinal mucositis.

54. The method according to claim 52, wherein the subject is afflicted with head and neck, breast, lung, ovarian, prostate, lymphatic, leukemic, and/or gastrointestinal cancer.

55. A method for mobilizing cells from the bone marrow comprising administering to a subject in need thereof an effective amount of at least one compound of any one of claims 1 to 40 and optionally at least one pharmaceutically acceptable ingredient.

56. The method according to claim 55, wherein the cells are hematopoietic cells and/or tumor cells.
